(19) United States
(12) Patent Application Publication
(10) Pub. No.: US 2024/0043928 A1
(43) Pub. Date: Feb. 8, 2024

(54) BIOMARKERS FOR MYELODYSPLASTIC SYNDROME (MDS) AND METHODS OF USING THE SAME

(71) Applicant: Eisai R&D Management Co., Ltd., Tokyo (JP)

(72) Inventors: Antonio GUALBERTO, Boston, MA (US); Catherine SCHOLZ, North Reading, MA (US); Jianjun XIAO, Lexington, MA (US)

(21) Appl. No.: 18/034,859

(22) PCT Filed: Nov. 3, 2021
(86) PCT No.: PCT/US2021/057839

§ 371 Date: May 1, 2023

Related U.S. Application Data
(60) Provisional application No. 63/260,837, filed on Sep. 1, 2021.
(60) Provisional application No. 63/109,730, filed on Nov. 4, 2020.

(51) Int. Cl. C12Q 1/6883; A61K 31/496; A61P 35/02

(52) U.S. Cl. CPC C12Q 1/6883; A61K 31/496; A61P 35/02; C12Q 2600/106; C12Q 2600/156

(57) ABSTRACT

The present disclosure relates to the treatment of transfusion dependence in myelodysplastic syndrome (MDS). The present disclosure relates to methods of using novel biomarkers to treat transfusion dependence in an MDS patient in need thereof. The present disclosure also relates to methods of identifying MDS patients suitable for treatment with a splicing modulator and/or predicting or monitoring treatment efficacy in an MDS patient. In some embodiments, the methods disclosed herein comprise determining at least the ratio of aberrant junction to canonical junction TMEM14C transcripts (TMEM14C AJ/CJ ratio) in the patient. In some embodiments, the methods disclosed herein comprise administering a therapeutically effective amount of a splicing modulator (e.g., Compound 1) based on the patient's TMEM14C AJ/CJ ratio. Therapeutic uses and compositions are also disclosed.

This Application claims the benefit of and priority to U.S. Provisional Application No. 63/109,730 filed Nov. 4, 2020, and U.S. Provisional Application No. 63/260,837, filed Sep. 1, 2021, the contents of both of which are expressly incorporated herein by reference in their entirety.

The instant application contains a Sequence Listing which has been submitted electronically in ASCII format and is hereby incorporated by reference in its entirety. Said ASCII copy, created on Oct. 27, 2021, is named 15647_0016-00304_SL.txt and is 21,994 bytes in size.

The present disclosure relates to the treatment of transfusion dependence in myelodysplastic syndrome (MDS). The present disclosure provides, in some embodiments, methods of using novel biomarkers to treat transfusion dependence in an MDS patient in need thereof. The present disclosure also provides, in some embodiments, methods of identifying MDS patients suitable for treatment with a splicing modulator (e.g., Compound 1) and/or predicting or monitoring treatment efficacy in an MDS patient. In some embodiments, the methods disclosed herein comprise determining the ratio of aberrant junction to canonical junction TMEM14C transcripts (TMEM14C AJ/CJ ratio) in the patient. In some embodiments, the methods disclosed herein comprise administering a therapeutically effective amount of a splicing modulator (e.g., Compound 1) based on the patient's TMEM14C AJ/CJ ratio. Therapeutic uses and compositions are also provided.

Myelodysplastic syndrome (MDS) is a collection of hematological conditions related to cancer and caused by abnormal blood-forming cells in bone marrow. These abnormal blood-forming cells form defective blood cells that die prematurely or are destroyed, leading to a cell shortage. Most commonly, MDS results in a shortage of red blood cells, but other types of blood cells can be affected.

Patients with MDS often develop severe anemia and require frequent blood transfusions, which can have clinical, health-related quality of life, and economic consequences (Balducci, Cancer. 2006;106:2087-2094; Almeida et al. Leuk Res. 2017;52:50-57). In addition to complications related to iron toxicity, alloimmunization, allergic reactions, and transfusion-transmitted infections can also occur (Sanz et al. Transfusion. 2013;53:710-715; Kimura et al. Blood Transfus. 2014;12:103-106; Koutsavlis, Anemia. 2016;2016:8494738; Singhal et al. Haematologica. 2017;102:2021-2029). Dependence on transfusions may also negatively impact survival and affect physical, functional, and social well-being (Harnan et al. Acta Haematol. 2016;136:23-42; Lucioni et al. Am J Blood Res. 2013;3:246-259; Stauder et al. Leukemia. 2018;32:1380-1392; Szende et al. Health Qual Life Outcomes. 2009;7:81). For instance, chronic blood transfusions can be time-consuming for the patient and may pose a psychosocial burden on patients and their families (Balducci, Cancer. 2006;106:2087-2094). In addition to these clinical and health-related quality of life burdens, transfusion-dependent patients may pose an economic burden as well. Long-term dependence on transfusions is associated with increased healthcare costs, especially for patients needing a higher transfusion frequency (DeZern et al. Leuk Lymphoma. 2017;58:2649-2656).

There are currently multiple drug therapies approved in the U.S. for MDS-related indications, including the parenterally administered recombinant erythropoietin drugs known as erythropoietin-stimulating agents; the nucleoside analog DNA methyltransferase inhibitors (hypomethylating agents) azacitidine and decitabine; luspatercept, the recombinant fusion protein that binds transforming growth factor β superfamily ligands; and the orally administered immunomodulatory agent lenalidomide. These drug therapies can induce hematologic improvement, but are not curative, and are also associated with treatment-emergent cytopenias and other adverse events. Thus, there remains a need for improved treatments for MDS, particularly those that can reduce transfusion dependence and minimize undesirable toxicities.

Accumulating evidence has linked various human diseases to dysregulation in RNA splicing, which can lead to differential exon inclusion/exclusion, intron retention, or usage of cryptic splice sites (Scotti and Swanson (2016) Nat Rev Genet. 17(1):19-32; Seiler et al. (2018) Cell Rep. 23(1):282-96). Several studies have documented alterations in the splicing profile of cancer cells, as well as in the splicing factors themselves (Agrawal et al. (2018) Curr Opin Genet Dev. 48:67-74). Altogether, these events account for functional changes that may contribute to tumorigenesis or resistance to therapy (Zhang and Manley, Cancer Discov. 2013;3(11):1228-1237; Siegfried and Karni, (2018) Curr Opin Genet Dev. 48:16-21).

Somatic mutations in splicing factor genes such as SF3B1, U2AF1, and SRSF2 affect approximately 50% of MDS patients and result in diverse alternative and aberrant mRNA splicing changes. Mutated SF3B1 has been associated with the ring sideroblast phenotype in MDS, which is typically characterized by defects in heme biosynthesis and iron accumulation in mitochondria. Aberrant splicing of genes involved in heme biosynthesis and iron metabolism, such as TMEM14C, ABCB7, and PPOX, has also been observed in MDS patients, particularly in those carrying SF3B1 mutations (Shiozawa et al. Nat Commun. 2018;9:3649). Aberrant splicing and downregulation of ABCB7 and PPOX has been reported in SF3B1 mutant cells and patients, and may contribute to defective erythropoiesis (see, e.g., Shiozawa et al. Nat Commun. 2018;9:3649, e.g., at FIG. 7e-g; Darman et al. Cell Reports. 2015;13:1033-1045, e.g., at FIG. 6D; Nikpour et al. Br J Haematol. 2010;149(6):844-854, e.g., at FIG. 1). Moreover, mutations in splicing factor genes have been reported to be one of the initiating events in the origin of MDS and other myeloid malignancies (Walter et al. N Engl J Med. 2012;366(12):1090-1098; Yoshida and Ogawa, Wiley Interdiscip Rev RNA. 2014;5(4):445-459).

Certain small molecules can modulate RNA splicing in malignant cells by promoting intron retention and/or exon skipping (Teng et al. (2017) Nat Commun. 8:15522). Compound 1 is a small molecule that binds to the SF3b complex and induces alternative splicing changes (Finci et al. Genes Dev. 2018;32(3-4):309-320; Lee et al. Nat Med. 2016;22(6):672-678). Compound 1 shows growth inhibitory activity in a panel of human acute myeloid leukemia (AML) cell lines, including mutant U2AF1, SRSF2, and SF3B1 cells, and oral administration of Compound 1 induces in vivo antitumor activity in xenograft models of leukemia expressing mutant SF3B1 (Seiler et al. Nat Med. 2018;24(4):497-504). Compound 1 has also been evaluated in humans for the treatment of myeloid cancers, but has shown inconsistent results across patients (NCT02841540; Steensma et al. Blood (2019) 134 (Supplement 1): 673).

Thus, improved therapies with splicing modulators such as Compound 1, as well as methods for identifying patients most likely to respond or benefit from such treatments, are needed. Biomarker-based strategies for selecting MDS patients likely to develop transfusion independence during treatment would be particularly desirable. Such strategies could inform therapeutic regimens, improve treatment efficacy, and/or improve quality of life for MDS patients.

The present disclosure is based, at least in part, on the surprising discovery that although splicing modulators such as Compound 1 have shown some inconsistent results in terms of anti-tumor responses in myeloid cancers (NCT02841540; Steensma et al. Blood (2019) 134 (Supplement 1): 673), that compound is surprisingly effective in reducing red blood cell transfusion dependence among MDS patients expressing elevated ratios of aberrantly spliced transcripts of the mitochondrial porphyrin transporter TMEM14C, a gene involved in erythropoiesis. Elevated pre-treatment expression of TMEM14C is associated with transfusion independence in MDS patients treated with Compound 1. Compound 1 may reduce or inhibit TMEM14C aberrant splicing in MDS patients. Without being bound by theory, it appears that MDS patients who exhibit transfusion independence following treatment with Compound 1, particularly MDS patients with SF3B1 mutations, may have high levels of TMEM14C aberrant transcripts prior to treatment with Compound 1 and transient downregulation during treatment.

Although SF3B1 mutant proteins affect many genes, including genes such as ZDHHC16, SLTM, SNURF, ZNF561, TAK1, ZNF410, and others involved in blood cell synthesis and metabolism, those genes generally do not correlate with transfusion independence. The present disclosure, in contrast, focuses on the surprising discovery that modulation of TMEM14C aberrant splicing in certain MDS patients renders those patients uniquely susceptible to transfusion independence after treatment with a splicing modulator such as Compound 1. In particular, an elevated pre-treatment ratio of aberrant junction to canonical junction TMEM14C transcripts (TMEM14C AJ/CJ ratio) may be useful to identify MDS patients likely to achieve transfusion independence during treatment with Compound 1. Such patients may also have a low level of TMEM14C expression (e.g., a level lower than in a healthy subject). In some embodiments, an elevated TMEM14C AJ/CJ ratio, alone or in combination with a low level of TMEM14C expression, is used as a biomarker to predict or determine whether a patient is likely to respond or benefit from treatment with Compound 1. In some embodiments, an elevated TMEM14C AJ/CJ ratio is a TMEM14C AJ/CJ ratio exceeding the ratio in a control (e.g., a control subject who does not have MDS). In some embodiments, an elevated TMEM14C AJ/CJ ratio is measured using one or more methods for detecting and quantifying nucleic acids, such as any of the exemplary methods described herein. In some embodiments, an elevated TMEM14C AJ/CJ ratio is measured using a PCR-based method such as real-time PCR (RT-PCR). In some embodiments, an elevated TMEM14C AJ/CJ ratio is measured using nucleic acid barcoding. In some embodiments, an elevated TMEM14C AJ/CJ ratio is a ratio exceeding the ratio in a control, as measured by nucleic acid barcoding. In some embodiments, an elevated TMEM14C AJ/CJ ratio is a ratio greater than about 4, e.g., 3.5, 3.6, 3.7, 3.8, 3.9, 4.0, 4.1, 4.2, 4.3, 4.4, or 4.5, as measured by nucleic acid barcoding.

In some embodiments, the present disclosure provides a TMEM14C AJ/CJ ratio for use as a biomarker for selecting patients to treat for transfusion dependence in a patient with MDS, e.g., by administering Compound 1. A TMEM14C AJ/CJ ratio may be evaluated alone or in combination with one or more additional biomarkers to identify the patient for treatment. For instance, aberrant splicing of other genes involved in heme biosynthesis and iron metabolism, including ABCB7 and/or PPOX, may likewise increase the susceptibility of certain MDS patients to transfusion independence after treatment with a splicing modulator such as Compound 1.

Downregulation of the iron exporter ABCB7 has been linked to increased mitochondrial iron accumulation observed in MDS patients with ring sideroblasts (Maio et al. Haematologica 2019;104:1756-1767). Loss of ABCB7 expression in experimental models has also been shown to cause defective heme biosynthesis, mitochondrial iron overload, and apoptosis of erythroid progenitors (Maio et al. Haematologica 2019;104:1756-1767). In some embodiments, an elevated pre-treatment ratio of aberrant junction to canonical junction ABCB7 transcripts (ABCB7AJ/CJ ratio) may be useful to identify MDS patients likely to achieve transfusion independence during treatment with Compound 1, e.g., when used alone or in combination with another biomarker such as an elevated TMEM14C AJ/CJ ratio. Likewise, PPOX may facilitate the mitochondrial transport of porphyrins (Shiozawa et al. Nat Commun. 2018;9:3649). In some embodiments, an elevated pre-treatment ratio of aberrant junction to canonical junction PPOX transcripts (PPOX AJ/CJ ratio) may be useful to identify MDS patients likely to achieve transfusion independence during treatment with Compound 1, e.g., when used alone or in combination with another biomarker such as an elevated TMEM14C AJ/CJ ratio.

In some embodiments, the splicing modulator is a pladienolide pyridine compound having Formula I or a pharmaceutically acceptable salt thereof, herein referred to as “Compound 1”:

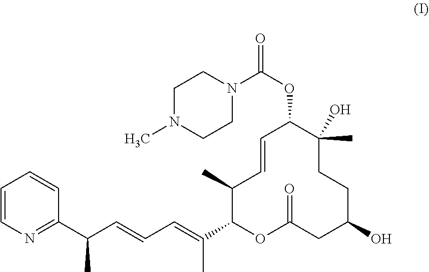 [SMILES: C/C(=C\C=C\[C@@H](C)c1ccccn1)[C@H]1OC(=O)C[C@H](O)CC[C@@](C)(O)[C@@H](OC(=O)N2CCN(C)CC2)/C=C/[C@@H]1C]

(also known as (2S,3S,4E,6S,7R,10R)-7,10-dihydroxy-3,7-dimethyl-12-oxo-2-[(2E,4E,6R)-6-(pyridine-2-yl)hepta-2,4-dien-2-yl]oxacyclododec-4-en-6-yl-4-methylpiperazine-1-carboxylate).

In some embodiments, the present disclosure provides methods of using novel biomarkers to treat transfusion dependence in a patient with MDS. The present disclosure also provides, in some embodiments, methods of identifying MDS patients suitable for treatment with a splicing modulator (e.g., Compound 1) and/or predicting or monitoring treatment efficacy in an MDS patient. In some embodiments, the methods disclosed herein comprise determining the ratio of aberrant junction to canonical junction TMEM14C transcripts (TMEM14C AJ/CJ ratio) in the patient. In some embodiments, the methods disclosed herein comprise administering a therapeutically effective amount of a splicing modulator (e.g., Compound 1) based on the patient's TMEM14C AJ/CJ ratio. Therapeutic uses and compositions are also disclosed.

In some embodiments, the present disclosure provides a method of treating transfusion dependence in a patient with MDS, comprising administering a therapeutically effective amount of Compound 1 to the transfusion-dependent MDS patient who has an elevated TMEM14C AJ/CJ ratio. In some embodiments, the present disclosure provides a method of treating transfusion dependence in a patient with MDS, comprising: (a) determining that the transfusion-dependent MDS patient has an elevated TMEM14C AJ/CJ ratio; and (b) administering a therapeutically effective amount of Compound 1 to the patient. In some embodiments, the present disclosure provides a method of identifying a transfusion-dependent MDS patient suitable for treatment with Compound 1, comprising: (a) determining that the patient has an elevated TMEM14C AJ/CJ ratio; and (b) identifying the patient as suitable for treatment with Compound 1. In some embodiments of the methods disclosed herein, the patient or a biological sample from the patient has a low level of TMEM14C expression. In some embodiments, Compound 1 reduces or inhibits TMEM14C aberrant splicing in the patient.

In some embodiments, the present disclosure provides a method of monitoring treatment efficacy in a transfusion-dependent MDS patient, comprising: (a) determining that the patient has an elevated TMEM14C AJ/CJ ratio; (b) administering a therapeutically effective amount of Compound 1 to the patient; and (c) determining the TMEM14C AJ/CJ ratio in the patient after administration, wherein a reduction in the TMEM14C AJ/CJ ratio after administration indicates an effective treatment. In some embodiments, the TMEM14C AJ/CJ ratio remains elevated after step (c), and the method further comprises administering an additional dose of Compound 1 to the patient. In some embodiments, the method further comprises administering additional doses of Compound 1 to the patient until the TMEM14C AJ/CJ ratio is no longer elevated. In some embodiments of the methods disclosed herein, the patient or a biological sample from the patient has a low level of TMEM14C expression. In some embodiments, Compound 1 reduces or inhibits TMEM14C aberrant splicing in the patient.

In some embodiments, determining an elevated AJ/CJ ratio comprises obtaining a biological sample from the patient and determining a TMEM14C AJ/CJ ratio in the sample. An exemplary canonical sequence for TMEM14C is CCGGGGCCTTCGTGAGACCGGTGCAGG CCTGGGGTAGTCT (SEQ ID NO: 1) (gene: TMEM14C; junction: chr6:10723474-10724802). An exemplary aberrant sequence for TMEM14C is CCGGGGCCTTCGTGAGACCGCTTG TTTTCTGCAGGTGCAG (SEQ ID NO: 2) (gene: TMEM14C; junction; chr6:10723474-10724788). Additional aberrant TMEM14C sequences are described in Darman et al. (Cell Reports. 2015;13:1033-1045, e.g., at Table S5), which is incorporated herein by reference for the disclosure of such sequences. In some embodiments, the biological sample comprises a blood sample or a bone marrow sample. In some embodiments, the blood sample comprises peripheral blood or plasma. In some embodiments, the bone marrow sample comprises a bone marrow aspirate or a bone marrow biopsy. In some embodiments, the biological sample comprises a urine sample. In some embodiments, the TMEM14C AJ/CJ ratio is determined by measuring RNA transcripts in the patient or in a biological sample from the patient. In some embodiments, measuring RNA transcripts comprises nucleic acid barcoding and/or real-time polymerase chain reaction (RT-PCR). In some embodiments, measuring RNA transcripts comprises nucleic acid barcoding.

In some embodiments, an elevated TMEM14C AJ/CJ ratio is a ratio exceeding the ratio in a control (e.g., in a control subject who does not have MDS). In some embodiments, an elevated TMEM14C AJ/CJ ratio is measured using one or more methods for detecting and quantifying nucleic acids, such as any of the exemplary methods described herein (e.g., PCR-based methods such as real-time PCR (RT-PCR), nucleic acid barcoding, etc.). In some embodiments, an elevated TMEM14C AJ/CJ ratio is a ratio exceeding the ratio in a control, as measured by nucleic acid barcoding. In some embodiments, an elevated TMEM14C AJ/CJ ratio is a ratio greater than about 1, about 2, about 4, about 10, about 15, about 20, or about 30, e.g., as measured by RNA expression quantification methods such as real-time reverse transcription PCR or nucleic acid barcoding. In some embodiments, an elevated TMEM14C AJ/CJ ratio is a ratio greater than about 4, as measured by nucleic acid barcoding.

In some embodiments, an elevated pre-treatment ratio of aberrant junction to canonical junction ABCB7 transcripts (ABCB7 AJ/CJ ratio) may be useful to identify MDS patients likely to achieve transfusion independence during treatment with Compound 1.

In some embodiments, the present disclosure provides a method of treating transfusion dependence in a patient with MDS, comprising administering a therapeutically effective amount of Compound 1 to the transfusion-dependent MDS patient who has an elevated ABCB7 AJ/CJ ratio. In some embodiments, the present disclosure provides a method of treating transfusion dependence in a patient with MDS, comprising: (a) determining that the transfusion-dependent MDS patient has an elevated ABCB7 AJ/CJ ratio; and (b) administering a therapeutically effective amount of Compound 1 to the patient. In some embodiments, the present disclosure provides a method of identifying a transfusion-dependent MDS patient suitable for treatment with Compound 1, comprising: (a) determining that the patient has an elevated ABCB7 AJ/CJ ratio; and (b) identifying the patient as suitable for treatment with Compound 1.

In some embodiments, determining an elevated AJ/CJ ratio comprises obtaining a biological sample from the patient and determining an ABCB7 AJ/CJ ratio in the sample. In some embodiments, the ABCB7 AJ/CJ ratio is determined by measuring RNA transcripts in the patient or in a biological sample from the patient. In some embodiments, an elevated ABCB7 AJ/CJ ratio is an ABCB7 AJ/CJ ratio exceeding the ratio in a control (e.g., a control subject who does not have MDS). In some embodiments, an elevated ABCB7 AJ/CJ ratio is measured using one or more methods for detecting and quantifying nucleic acids, such as any of the exemplary methods described herein. In some embodiments, an elevated ABCB7 AJ/CJ ratio is measured using a PCR-based method such as real-time PCR (RT-PCR). In some embodiments, an elevated ABCB7 AJ/CJ ratio is measured using nucleic acid barcoding. In some embodiments, an elevated ABCB7 AJ/CJ ratio is a ratio exceeding the ratio in a control, as measured by nucleic acid barcoding.

In some embodiments, an elevated pre-treatment ratio of aberrant junction to canonical junction PPOX transcripts (PPOX AJ/CJ ratio) may be useful to identify MDS patients likely to achieve transfusion independence during treatment with Compound 1.

In some embodiments, the present disclosure provides a method of treating transfusion dependence in a patient with MDS, comprising administering a therapeutically effective amount of Compound 1 to the transfusion-dependent MDS patient who has an elevated PPOX AJ/CJ ratio. In some embodiments, the present disclosure provides a method of treating transfusion dependence in a patient with MDS, comprising: (a) determining that the transfusion-dependent MDS patient has an elevated PPOX AJ/CJ ratio; and (b) administering a therapeutically effective amount of Compound 1 to the patient. In some embodiments, the present disclosure provides a method of identifying a transfusion-dependent MDS patient suitable for treatment with Compound 1, comprising: (a) determining that the patient has an elevated PPOX AJ/CJ ratio; and (b) identifying the patient as suitable for treatment with Compound 1.

In some embodiments, determining an elevated AJ/CJ ratio comprises obtaining a biological sample from the patient and determining an PPOX AJ/CJ ratio in the sample. In some embodiments, the PPOX AJ/CJ ratio is determined by measuring RNA transcripts in the patient or in a biological sample from the patient. In some embodiments, an elevated PPOX AJ/CJ ratio is a PPOX AJ/CJ ratio exceeding the ratio in a control (e.g., a control subject who does not have MDS). In some embodiments, an elevated PPOX AJ/CJ ratio is measured using one or more methods for detecting and quantifying nucleic acids, such as any of the exemplary methods described herein. In some embodiments, an elevated PPOX AJ/CJ ratio is measured using a PCR-based method such as real-time PCR (RT-PCR). In some embodiments, an elevated PPOX AJ/CJ ratio is measured using nucleic acid barcoding. In some embodiments, an elevated PPOX AJ/CJ ratio is a ratio exceeding the ratio in a control, as measured by nucleic acid barcoding.

In some embodiments, determining an elevated AJ/CJ ratio comprises determining more than one AJ/CJ ratio, e.g., two, three, or more AJ/CJ ratios, e.g., in a biological sample from the patient.

In some embodiments, determining an elevated AJ/CJ ratio comprises determining a TMEM14C AJ/CJ ratio and an ABCB7 AJ/CJ ratio. In some embodiments, determining an elevated AJ/CJ ratio comprises obtaining a biological sample from the patient and determining a TMEM14C AJ/CJ ratio and an ABCB7 AJ/CJ ratio in the sample.

In some embodiments, determining an elevated AJ/CJ ratio comprises determining a TMEM14C AJ/CJ ratio and a PPOX AJ/CJ ratio. In some embodiments, determining an elevated AJ/CJ ratio comprises obtaining a biological sample from the patient and determining a TMEM14C AJ/CJ ratio and a PPOX AJ/CJ ratio in the sample.

In some embodiments, determining an elevated AJ/CJ ratio comprises determining a TMEM14C AJ/CJ ratio, an ABCB7 AJ/CJ ratio, and a PPOX AJ/CJ ratio. In some embodiments, determining an elevated AJ/CJ ratio comprises obtaining a biological sample from the patient and determining a TMEM14C AJ/CJ ratio, an ABCB7 AJ/CJ ratio, and a PPOX AJ/CJ ratio in the sample.

In some embodiments, the MDS is MDS with multilineage dysplasia (MDS-MLD), MDS with single lineage dysplasia (MDS-SLD), MDS with ring sideroblasts (MDS-RS), MDS with excess blasts (MDS-EB), MDS associated with isolated del(5q), or MDS-unclassified (MDS-U). In some embodiments, the MDS is MDS of intermediate-1 risk or lower according to the International Prognostic Scoring System. In some embodiments, the MDS is MDS of intermediate-2 risk or lower according to the International Prognostic Scoring System. In some embodiments, the MDS is MDS-MLD. In some embodiments, the MDS is MDS-EB. In some embodiments, the MDS-EB is MDS-EB1 or MDS-EB2. In some embodiments, the MDS is MDS-EB2.

In some embodiments, the patient or a biological sample from the patient comprises a mutation in one or more genes associated with RNA splicing. In some embodiments, the patient or a biological sample from the patient comprises a mutation in one or more genes selected from SF3B1, SRSF2, U2AF1, and ZRSR2. In some embodiments, the patient or a biological sample from the patient comprises a mutation in SF3B1. In some embodiments, the mutation in SF3B1 comprises or consists of a mutation at one or more of positions E622, H662, K666, K700, R625, or V701 in SF3B1. In some embodiments, the mutation in SF3B1 comprises or consists of a mutation at one or more of positions H662, K700, or R625 in SF3B1. In some embodiments, the mutation in SF3B1 comprises or consists of a mutation at position K700 in SF3B1. In some embodiments, the mutation at position K700 is K700E and/or the mutation at position R625 is R625C. In some embodiments, the mutation in SF3B1 comprises K700E and/or R625C.

In some embodiments, Compound 1 is administered to the patient orally.

In some embodiments, Compound 1 is administered to the patient once daily. In some embodiments, Compound 1 is administered to the patient once daily on a 5 days on/9 days off dosing schedule. In some embodiments, Compound 1 is administered to the patient once daily on a 21 days on/7 days off dosing schedule. In some embodiments, Compound 1 is administered to the patient once daily on a continuous dosing schedule. In some embodiments, Compound 1 is administered to the patient once daily on a continuous dosing schedule until an adverse event or drug-related toxicity (e.g., rash, neutropenia, thrombocytopenia) is observed. In some embodiments, a treatment holiday is incorporated into a once daily dosing schedule, e.g., after an adverse event or drug-related toxicity event is observed. In some embodiments, a treatment holiday is incorporated after at least about 5 days (e.g., after about 5 days, after about 7 days, after about 14 days, after about 21 days, or more) of once daily continuous dosing. In some embodiments, Compound 1 is administered to the patient once daily for one or more 28-day cycles. In some embodiments, the therapeutically effective amount of Compound 1 is about 2 mg to about 20 mg given in a single dose on the day of administration. In some embodiments, the therapeutically effective amount of Compound 1 is about 2 mg, about 3.5 mg, about 5 mg, about 7 mg, about 10 mg, about 12 mg, about 14, or about 20 mg given in a single dose on the day of administration.

In some embodiments, Compound 1 is administered to the patient twice daily. In some embodiments, Compound 1 is administered to the patient twice daily on a 5 days on/9 days off dosing schedule. In some embodiments, Compound 1 is administered to the patient twice daily on a 21 days on/7 days off dosing schedule. In some embodiments, Compound 1 is administered to the patient twice daily on a continuous dosing schedule. In some embodiments, Compound 1 is administered to the patient twice daily on a continuous dosing schedule until an adverse event or drug-related toxicity is observed. In some embodiments, a treatment holiday is incorporated into a twice daily dosing schedule. In some embodiments, a treatment holiday is incorporated after at least about 5 days (e.g., after about 5 days, after about 7 days, after about 14 days, after about 21 days, or more) of twice daily continuous dosing. In some embodiments, Compound 1 is administered to the patient twice daily for one or more 28-day cycles. In some embodiments, the therapeutically effective amount of Compound 1 is a total of about 2 mg to about 20 mg given in two divided doses on the day of administration. In some embodiments, the therapeutically effective amount of Compound 1 is about 10 mg, about 15 mg, or about 20 mg given in two divided doses on the day of administration. In some embodiments, the first and second dose is each independently about 2 mg to about 10 mg. In some embodiments, the first and second dose is each independently about 5 mg to about 10 mg. In some embodiments, the first dose is about 2 mg to about 5 mg and the second dose is about 7 mg to about 10 mg. In some embodiments, the first dose is about 7 mg to about 10 mg and the second dose is about 2 mg to about 5 mg. In some embodiments, the first dose is about 10 mg and the second dose is about 5 mg. In some embodiments, the first dose is about 5 mg and the second dose is about 10 mg. In some embodiments, the first dose and the second dose are each about 5 mg. In some embodiments, the first dose and the second dose are each about 7.5 mg. In some embodiments, the first dose and the second dose are each about 10 mg.

In some embodiments, the dose of Compound 1 administered to the patient is reduced over time. For example, at the start of treatment, Compound 1 may be administered at a dose of about 10 mg given twice daily, i.e., the first dose and the second dose are each about 10 mg. In some embodiments, the interval between the first dose and the second dose is about 8 to about 16 hours, e.g., about 10 hours to about 14 hours (e.g., about 10 hours, about 11 hours, about 12 hours, about 13 hours, about 14 hours). This daily dosing may then be reduced one or more times, e.g., after an adverse event or drug-related toxicity event is observed. In some embodiments, a first dose reduction comprises a first dose of about 5 mg and a second dose of about 10 mg, or vice versa. In some embodiments, a second or a subsequent dose reduction comprises a first dose and a second dose that are each about 5 mg.

In some embodiments, treatment with Compound 1 reduces or eliminates the patient's transfusion dependence. In some embodiments, treatment with Compound 1 reduces the number or frequency of transfusions given to the patient by at least about 10%, about 20%, about 30%, about 40%, about 50%, or about 60% as compared to the number or frequency prior to treatment. In some embodiments, treatment with Compound 1 reduces the number or frequency of transfusions given to the patient by at least about 30% as compared to the number or frequency prior to treatment. In some embodiments, treatment with Compound 1 reduces the number or frequency of transfusions given to the patient by at least about 60% as compared to the number or frequency prior to treatment. In some embodiments, the reduction in the number or frequency of transfusions observed with Compound 1 is greater than the reduction observed with an alternate treatment. In some embodiments, the time period between transfusions observed with Compound 1 is longer than the time period between transfusions observed with an alternate treatment. Exemplary alternate treatments include lenalidomide (see, e.g., List et al. (N Engl J Med. 2006;355(14):1456-1465), Fenaux et al. (Blood. 2011;118(14):3765-3776)) and luspatercept (see, e.g., Fenaux et al. (N Engl J Med. 2020;382(2):140-151)). In some embodiments, the reduction in the number or frequency of transfusions is measured over a period of at least 56 consecutive days (8 weeks), wherein the period begins any time after the start of treatment. In some embodiments, the patient does not receive any transfusions for a period of at least 56 consecutive days (8 weeks), wherein the period begins any time after the start of treatment. In some embodiments, the reduction in the number or frequency of transfusions is measured over a period of at least 8 weeks or more during the first 24 weeks of treatment. In some embodiments, the reduction in the number or frequency of transfusions is measured over a period of at least 12 weeks or more during the first 24 weeks of treatment. In some embodiments, the reduction in the number or frequency of transfusions is measured over a period of at least 12 weeks or more during the first 48 weeks of treatment. In some embodiments, the reduction in the number or frequency of transfusions is measured over a period of at least 16 weeks or more during the first 24 weeks of treatment. In some embodiments, the reduction in the number or frequency of transfusions is measured over a period of at least 16 weeks or more during the first 48 weeks of treatment. In some embodiments, the transfusions comprise red blood cell (RBC) transfusions, platelet transfusions, or both. In some embodiments, the transfusions comprise RBC transfusions. In some embodiments, treatment with Compound 1 increases the amount of bone marrow sideroblasts in the patient as compared to the amount prior to treatment. In some embodiments, treatment with Compound 1 increases the amount of bone marrow sideroblasts in the patient by at least about 10%, about 20%, about 30%, or about 40% as compared to the amount prior to treatment.

### BRIEF DESCRIPTION OF THE DRAWINGS

FIG. 1A-1C show swimmer plots of enrolled patients and their duration on therapy by disease subtype and spliceosome missense mutation at baseline: lower-risk myelodysplastic syndrome (MDS) or chronic myelomonocytic leukemia (CMML) (FIG. 1A), higher-risk MDS or CMML (FIG. 1B), and acute myeloid leukemia (AML) (FIG. 1C). Colors designate dose level of enrolled patients. *, CMML patient. Abbreviation: QD, once daily.

FIG. 2 shows mean plasma concentrations of Compound 1 (ng/mL) on Cycle 1 Day 4 (depicted for Schedule I). Abbreviation: h, hours.

FIG. 3A-3C show pre-treatment TMEM14C AJ/CJ and red blood cell transfusion independence (RBC TI) for MDS patients treated with Compound 1. FIG. 3A shows box plots indicating the relationship between pre-treatment TMEM14C AJ/CJ and RBC TI on study by tumor indication. Diagnosis of AML, MDS, or CMML is shown. FIG. 3B shows receiver operating curve (ROC) analyses and ranking of MDS patients with available pre-treatment TMEM14C AJ/CJ data. FIG. 3C shows TMEM14C AJ/CJ ratios following Compound 1 treatment in MDS patients who had elevated pre-treatment TMEM14C AJ/CJ.

FIG. 4 shows an exemplary study design (NCT02841540). *, Eligible patients had adequate vision and organ function, defined as: creatinine ≤1.7 mg/dL or calculated creatine clearance (CrCl) ≥50 mL/min; direct bilirubin ≤1.5 times upper limit of normal (ULN); alanine aminotransferase and aspartate aminotransferase (AST/ALT) ≤3.0 times ULN; albumin ≥2.5 mg/dL; normal vitamin A; and vision correction to 20/40 unless due to cataracts. Abbreviations: ALT, alanine aminotransferase; AML, acute myeloid leukemia; AST, aspartate aminotransferase; CMML, chronic myelomonocytic leukemia; CrCl, creatinine clearance; MDS, myelodysplastic syndrome; ULN, upper limit of normal.

FIG. 5 shows a heat map indicating a Compound 1 dose-dependent modulation of relative expression of splicing markers. Abbreviation: ES, exon skipping.

FIG. 6 shows RT-qPCR gene expression in residual samples. Box plots represent gene expression by RT-qPCR for patient subsets. Kruskal Wallis test was used to determine differences between groups. p values for RBC TI yes vs. no were 0.055 and 0.025 for the TMEM14C AJ/CJ ratio at Cycle 1 Day 1 and Cycle 1 Day 4, respectively. Abbreviation: RT-qPCR, quantitative reverse transcription PCR.

FIG. 7 shows mutations in patients experiencing RBC TI. Mutations were detected pre-treatment on Cycle 1 Day 1 in peripheral blood of patients who experienced RBC TI periods with Compound 1 treatment.

FIG. 8A-8B show box plots indicating the relationship between pre-treatment ABCB7 expression (as determined by RT-PCR) and SF3B1 mutation (FIG. 8A), or RBC TI on study (FIG. 8B), in patients who received treatment with Compound 1. RBC TI “on study” refers to RBC TI experienced by a patient while participating in the study, e.g., while on Compound 1 treatment or during treatment follow up.

### DETAILED DESCRIPTION

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed to limit the scope of the present disclosure.

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

All references cited herein, including, but not limited to, published and unpublished patent applications, granted patents, and literature references, are incorporated herein by reference and are hereby made a part of this specification. To the extent a cited reference conflicts with the disclosure herein, the specification shall control.

As used herein, the singular forms of a word also include the plural form, unless the context clearly dictates otherwise; as examples, the terms “a,” “an,” and “the” are understood to be singular or plural. By way of example, “an element” means one or more element. The term “or” shall mean “and/or” unless the specific context indicates otherwise.

The term “Compound 1,” as used herein, refers to at least one entity chosen from the compound of Formula I and pharmaceutically acceptable salts thereof. Furthermore, unless otherwise stated, “Compound 1” may be one or more of the enantiomeric, diastereomeric, and/or geometric (or conformational) forms of the compound(s); for example, the R and S configurations for each asymmetric center, (Z) and (E) double bond isomers, and (Z) and (E) conformational isomers. Unless otherwise stated, compounds depicted herein coexisting with tautomeric forms are within the scope of the disclosure. Additionally, unless otherwise stated, structures depicted herein are also meant to include compounds that differ only in the presence of one or more isotopically enriched atoms. For example, compounds having the depicted structures except for the replacement of hydrogen by deuterium or tritium, or the replacement of a carbon by a ¹³C- or ¹⁴C-enriched carbon are within the scope of this disclosure. Such compounds may be useful, for example, as analytical tools or probes in biological assays. Unless otherwise stated, administration or use of Compound 1 includes its administration or use in combination with any suitable vehicles or excipients, e.g., as formulated for a desired route of administration.

Formula I may be represented by the following:

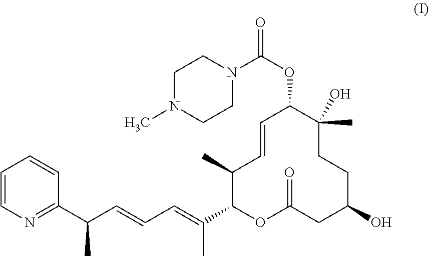 [SMILES: C/C(=C\C=C\[C@@H](C)c1ccccn1)[C@H]1OC(=O)C[C@H](O)CC[C@@](C)(O)[C@@H](OC(=O)N2CCN(C)CC2)/C=C/[C@@H]1C]

and/or the chemical name (2S,3S,4E,6S,7R,10R)-7,10-dihydroxy-3,7-dimethyl-12-oxo-2-[(2E,4E,6R)-6-(pyridine-2-yl)hepta-2,4-dien-2-yl]oxacyclododec-4-en-6-yl-4-methylpiperazine-1-carboxylate.

The term “pharmaceutically acceptable” means approved or approvable by a regulatory agency of the Federal or a state government or listed in the U.S. Pharmacopeia or other generally recognized pharmacopeia, for use in animals, and more particularly in humans.

A “pharmaceutically acceptable salt” is a salt that retains a desired biological activity of the parent compound and does not impart undesired toxicological effects. Examples of such salts are: (a) acid addition salts formed with inorganic acids, for example, hydrochloric acid, hydrobromic acid, sulfuric acid, phosphoric acid, nitric acid and the like; and salts formed with organic acids, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. See, e.g., Haynes et al. “Commentary: Occurrence of Pharmaceutically Acceptable Anions and Cations in the Cambridge Structural Database,” J Pharmaceutical Sciences, vol. 94, no. 10 (2005), and Berge et al. “Pharmaceutical Salts,” J Pharmaceutical Sciences, vol. 66, no. 1 (1977), which are incorporated by reference herein.

A “therapeutic effective amount” of, e.g., Compound 1, is an amount sufficient to perform a specifically stated purpose, for example to produce a therapeutic effect after administration to a patient. In the case of MDS, a therapeutically effective amount of Compound 1 may reduce a patient's TMEM14C AJ/CJ ratio, reduce the number or frequency of transfusions given to a patient, increase the number of bone marrow sideroblasts in a patient, relieve one or more symptoms of MDS, and/or provide some other indicia of treatment efficacy. A “prophylactically effective amount” refers to an amount effective, at dosages and for periods of time necessary, to achieve the desired prophylactic result, for example to prevent or reduce a patient's risk of becoming dependent on transfusions. Typically, since a prophylactic dose is used in patients prior to or at an earlier stage of disease, the prophylactically effective amount will be less than the therapeutically effective amount.

As used herein, the term “treat” or “treatment” or “therapeutic” (and grammatically related terms) refers to any improvement of any consequence of disease, such as a reduction or elimination of transfusion dependence, prolonged survival, less morbidity, and/or a lessening of side effects which result from an alternative therapeutic modality. Full eradication of a disease or a symptom or consequence thereof is encompassed but not required for a treatment act. For example, treatment of transfusion dependence in a patient with MDS includes reducing the number and/or frequency of transfusions given to a patient but does not require eliminating the need for transfusions. Treatment may also refer to the administration of Compound 1 to a patient, e.g., a transfusion-dependent MDS patient. The treatment can be to prevent, cure, heal, alleviate, relieve, alter, remedy, ameliorate, palliate, improve or affect the disease, one or more symptoms or consequences of the disease, or the predisposition toward the disease, e.g., MDS or transfusion dependence associated with MDS. In some embodiments, the treatment reduces or eliminates a patient's dependence on transfusions.

The terms “subject” and “patient” are used interchangeably herein to refer to any animal, such as any mammal, including but not limited to, humans, non-human primates, rodents, and the like. In some embodiments, the subject or patient is a mammal. In some embodiments, the subject or patient is a human.

As used herein, a patient is “suitable for” or “in need of” a treatment if such patient would benefit biologically, medically, and/or in quality of life from such treatment. In some embodiments, a patient suitable for treatment with Compound 1 is a transfusion-dependent MDS patient who has a particular TMEM14C AJ/CJ ratio. In some embodiments, the TMEM14C AJ/CJ ratio is used as a biomarker to predict or determine whether a patient is likely to respond or benefit from treatment with Compound 1. In some embodiments, the patient has an elevated TMEM14C AJ/CJ ratio, e.g., a ratio exceeding the ratio in a control (e.g., in a control subject who does not have MDS). In some embodiments, an elevated TMEM14C AJ/CJ ratio is measured using one or more methods for detecting and quantifying nucleic acids, such as any of the exemplary methods described herein. In some embodiments, an elevated TMEM14C AJ/CJ ratio is measured using a PCR-based method such as real-time PCR (RT-PCR). In some embodiments, an elevated TMEM14C AJ/CJ ratio is measured using nucleic acid barcoding. In some embodiments, an elevated TMEM14C AJ/CJ ratio is a ratio exceeding the ratio in a control, as measured by nucleic acid barcoding. In some embodiments, an elevated TMEM14C AJ/CJ ratio is a ratio greater than about 4, as measured by nucleic acid barcoding. In some embodiments, the patient or a biological sample from the patient has a low level of TMEM14C expression. In some embodiments, Compound 1 reduces or inhibits TMEM14C aberrant splicing in the patient.

The term “splice variant,” as used herein, refers to nucleic acid sequences that span a junction either between two exon sequences or across an intron-exon boundary in a gene, where the junction can be alternatively spliced. Alternative splicing includes alternate 3′ splice site selection (“3′ss”), alternate 5′ splice site selection (“5′ss”), differential exon inclusion, exon skipping, and intron retention.

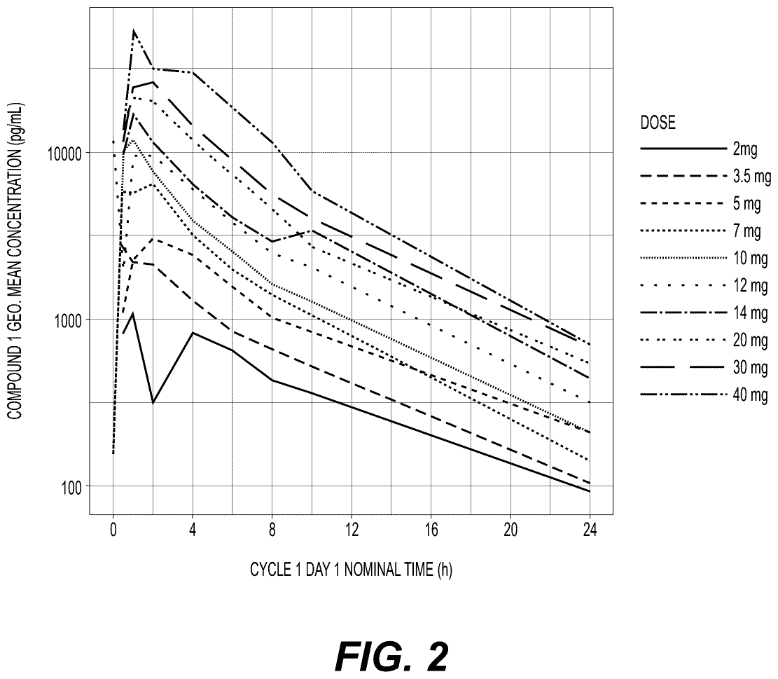

Certain splice variants associated with a given genomic location may be referred to as wild type or “canonical” splice variants. Wild type splice variants are variants most frequently found in the human population. These splice variants expressed as RNA transcripts may be referred to as “canonical junction” or “CJ” transcripts. An exemplary canonical sequence for TMEM14C is CCGGGGCCTTCGTGAGACCGGTGCAGGCCTGGGGT AGTCT (SEQ ID NO: 1) (gene: TMEM14C; junction: chr6:10723474-10724802). Examples of canonical splice sites are also provided herein. See, e.g., “AG” canonical 3′ splice sites shown in SEQ ID NO: 5 (TMEM14C) and SEQ ID NO: 6 (ABCB7). Additional non-limiting examples of canonical splice sites are described in Dolatshad et al. (Leukemia. 2016;30:2322-2331, e.g., at Supplemental FIG. 2), which is incorporated herein by reference for the disclosure of such sites.

Additional splice variants may be referred to as “aberrant” splice variants, which differ from the canonical splice variant. These splice variants expressed as RNA transcripts may be referred to as “aberrant junction” or “AJ” transcripts. An exemplary aberrant sequence for TMEM14C is CCGGGGCCTTCGTGAGACCGCTTGTTTTCTGCAGGTGCAG (SEQ ID NO: 2) (gene: TMEM14C; junction; chr6:10723474-10724788). Examples of aberrant splice sites are also provided herein. See, e.g., “AG” cryptic 3′ splice sites shown in SEQ ID NO: 5 (TMEM14C) and SEQ ID NO: 6 (ABCB7). Additional non-limiting examples of aberrant splice sites are described in Dolatshad et al. (Leukemia. 2016;30:2322-2331, e.g., at Supplemental FIG. 2), which is incorporated herein by reference for the disclosure of such sites.

The term “AJ/CJ ratio” refers to the ratio of aberrant junction to canonical junction transcripts of a particular gene or locus (e.g., TMEM14C). The term “TMEM14C AJ/CJ ratio” refers to the ratio of aberrant junction to canonical junction TMEM14C transcripts, e.g., in a patient or a sample from a patient. Exemplary methods for detecting and quantifying nucleic acids include nucleic acid barcoding, nanoparticle probes, in situ hybridization, microarray, nucleic acid sequencing, and PCR-based methods, including real-time PCR (RT-PCR). In some embodiments, a TMEM14C AJ/CJ ratio is determined by measuring RNA transcripts in a patient or in a sample from the patient (e.g., a blood sample, a bone marrow sample, and/or a urine sample). In some embodiments, measuring RNA transcripts comprises nucleic acid barcoding and/or RT-PCR. In some embodiments, a TMEM14C AJ/CJ ratio is determined using nucleic acid barcoding.

The term “elevated” when used to describe a TMEM14C AJ/CJ ratio in a patient or a sample from a patient means that the ratio of aberrant junction to canonical junction TMEM14C transcripts is greater than about 0.1 (e.g., greater than about 0.1, about 0.2, about 0.5, about 1, about 2, about 4, about 10, about 15, about 20, about 30, or more), e.g., as measured by nucleic acid barcoding. In some embodiments, an elevated TMEM14C AJ/CJ ratio is a ratio greater than about 4, as measured by nucleic acid barcoding (e.g., using a NanoString® assay (NanoString Technologies) as described in, e.g., U.S. Pat. Nos. 8,519,115; 7,919,237; and in Kulkarni (Current Protocols in Molecular Biology, 2011;94:25B.10.1-25B.10.17)).

The term “low” when used to describe TMEM14C expression in a patient or a sample from a patient means that the level is lower than in a healthy subject.

The term “ABCB7,” as used herein, denotes a gene which encodes ATP binding cassette subfamily B member 7, a membrane-associated protein and member of the superfamily of ATP-binding cassette (ABC) transporters. Exemplary ABCB7 sequences include but are not limited to: ABCB7, transcript variant 1 (UCSC: uc004ebz.4; RefSeq: NM_004299.6); ABCB7, transcript variant 2 (UCSC: uc004eca.4; RefSeq: NM_001271696.3); ABCB7, transcript variant 3 (UCSC: uc010nlt.4; RefSeq: NM_001271697.3); ABCB7, transcript variant 4 (UCSC: uc011mqn.3; RefSeq: NM_001271698.3); and ABCB7, transcript variant 5 (UCSC: uc010nls.4; RefSeq: NM_001271699.3). Exemplary primer sequences for ABCB7 include, for example, forward primer AATGAACAAAGCAGATAATGATGCAGG (SEQ ID NO: 7) and reverse primer TCCCTGACTGGCGAGCACCATTA (SEQ ID NO: 8). See, e.g., Shiozawa et al. Nat Commun. 2018;9(1):3649, e.g., at Supplementary Table 5, which is incorporated herein by reference for the disclosure of such primer sequences. Such primers may be used to detect ABCB7 expression. Primers can also be designed to detect splice variants of ABCB7 by those skilled in the art. In some embodiments, the primers described herein are used to detect ABCB7 expression (e.g., total ABCB7 expression) in a patient or a sample from a patient. In some embodiments, ABCB7 expression is measured using one or more methods for detecting and quantifying nucleic acids, such as any of the exemplary methods described herein. In some embodiments, ABCB7 expression is measured using a PCR-based method such as real-time PCR (RT-PCR).

The term “PPOX,” as used herein, denotes a gene encoding the penultimate enzyme of heme biosynthesis, which catalyzes the 6-electron oxidation of protoporphyrinogen IX to form protoporphyrin IX. Exemplary PPOX sequences include but are not limited to: PPOX, transcript variant 1 (RefSeq: NM_000309.5); PPOX, transcript variant 2 (RefSeq: NM_001122764.3); PPOX, transcript variant 3 (RefSeq: NM_001350128.2); PPOX, transcript variant 4 (RefSeq: NM_001350129.2); and PPOX, transcript variant 5 (RefSeq: NM_001350130.2). Exemplary primer sequences for PPOX include, for example, forward primer GGCCCTAATGGTGCTATCTTTG (SEQ ID NO: 9) and reverse primer CTTCTGAATCCAAGCCAAGCTC (SEQ ID NO: 10). See, e.g., Shiozawa et al. Nat Commun. 2018;9(1):3649, e.g., at Supplementary Table 5, which is incorporated herein by reference for the disclosure of such primer sequences. Such primers may be used to detect PPOX expression. Primers can also be designed to detect splice variants of PPOX by those skilled in the art. In some embodiments, the primers described herein are used to detect PPOX expression (e.g., total PPOX expression) in a patient or a sample from a patient. In some embodiments, PPOX expression is measured using one or more methods for detecting and quantifying nucleic acids, such as any of the exemplary methods described herein. In some embodiments, PPOX expression is measured using a PCR-based method such as real-time PCR (RT-PCR).

The term “SF3B1,” as used herein, denotes a gene which encodes subunit 1 of the splicing factor 3b protein complex. Splicing factor 3b is a component of the U2 small nuclear ribonucleoproteins complex (U2 snRNP), which binds to pre-mRNA at a region containing the branchpoint site and is involved in early recognition and stabilization of the spliceosome at the 3′ splice site (3′ss). In some embodiments, the wild-type human SF3B1 protein is as set forth in SEQ ID NO: 3 (GenBank Accession Number NP_036565, Version NP_036565.2) (Bonnal et al. Nature Review Drug Discovery 2012;11:847-859) or an encoding nucleic acid sequence is as set forth in SEQ ID NO: 4 (GenBank Accession Number NM_012433, Version NM_012433.4).

In some embodiments, SF3B1 mutations are determined at the protein or nucleic acid level as SF3B1 sequences that differ from the amino acid sequence of the human wild-type SF3B1 protein as set forth in SEQ ID NO: 3, or an encoding nucleic acid sequence as set forth in SEQ ID NO: 4. In some embodiments, one or more SF3B1 mutations include a point mutation (e.g., a missense or nonsense mutation), an insertion, and/or a deletion. In some embodiments, one or more SF3B1 mutations include a somatic mutation. In some embodiments, one or more SF3B1 mutations include a heterozygous mutation or a homozygous mutation. Exemplary SF3B1 mutations include mutations at one or more of positions E622, H662, K666, K700, R625, or V701 in SF3B1. In some embodiments, a mutation in SF3B1 comprises or consists of a mutation at one or more of positions H662, K700, or R625 in SF3B1. In some embodiments, a mutation in SF3B1 comprises or consists of a mutation at position K700 in SF3B1. In some embodiments, the mutation at position K700 is K700E. In some embodiments, the mutation at position R625 is R625C. In some embodiments, a mutation in SF3B1 comprises K700E and/or R625C.

Detection of mutations in spliceosome proteins, e.g., one or more mutations in SF3B1, SRSF2, U2AF1, and/or ZRSR2 (e.g., one or more mutations in SF3B1), can be determined at the protein or nucleic acid level using any method known in the art. A variety of methods exist for detecting, quantifying, and sequencing nucleic acids or their encoded proteins, and each may be adapted for detection of mutations (e.g., SF3B1 mutations) in the embodiments disclosed herein. Exemplary methods include an assay to quantify nucleic acids such as in situ hybridization, microarray, nucleic acid sequencing, PCR-based methods, including real-time PCR (RT-PCR), whole exome sequencing, single nucleotide polymorphism analysis, deep sequencing, targeted gene sequencing, or any combination thereof. In some embodiments, the foregoing techniques and procedures are performed according to methods described in, e.g., Sambrook et al. Molecular Cloning: A Laboratory Manual (3rd ed., Cold Spring Harbor Laboratory Press, Cold Spring Harbor, N.Y. (2000)).

The term “myelodysplastic syndrome” or “MDS,” as used herein, refers to a hematological disorder caused by poorly formed blood cells or blood cells that do not work properly. MDS may be characterized by one or more of the following: ineffective blood cell production, progressive cytopenia, risk of progression to acute leukemia or cellular marrow with impaired morphology, and maturation (dysmyelopoiesis). Symptoms often associated with MDS include but are not limited to anemia, thrombocytopenia, neutropenia, cytopenia, bicytopenia (two deficient cell types), and pancytopenia (three deficient cell types).

The term “MDS patient,” as used herein, refers to a patient who has been diagnosed with MDS according to the World Health Organization (WHO) 2008 classification (reviewed in Vardiman et al. Blood 2009;114(5):937-951). In some embodiments, the diagnosis is made or confirmed using a physical examination and/or one or more diagnostic tests. Exemplary tests used to diagnose MDS are described herein and include blood tests, peripheral (circulating) blood smears, bone marrow aspiration and biopsy, molecular testing, cytogenetic (chromosomal) analysis, and immunophenotyping. An MDS patient may be transfusion-dependent or transfusion-independent.

MDS may be divided into subtypes based on the type of blood cells involved (e.g., red blood cells, white blood cells, platelets). Subtypes of MDS, e.g., those according to the WHO 2008 classification, are described herein and include: MDS with single lineage dysplasia (MDS-SLD), MDS with multilineage dysplasia (MDS-MLD), MDS with ring sideroblasts (MDS-RS), MDS with excess blasts (MDS-EB), MDS associated with isolated del(5q), and MDS-unclassified (MDS-U).

MDS with single lineage dysplasia (MDS-SLD) may include and/or be referred to as refractory cytopenia with unilineage dysplasia (RCUD), refractory anemia (RA), refractory neutropenia (RN), and/or refractory thrombocytopenia (RT). MDS-SLD typically involves one blood cell type (e.g., red blood cells, white blood cells, platelets) being low in number and appearing abnormal under the microscope. In some embodiments, blood findings of MDS-SLD include one or more of: unicytopenia or bicytopenia and no or rare blasts (<1%). Bicytopenia may occasionally be observed; however, cases with pancytopenia are generally classified as MDS-U. In some embodiments, bone marrow findings of MDS-SLD include one or more of: unilineage dysplasia, ≥10% of the cells of the affected lineage are dysplastic, <5% blasts, and <15% of erythroid precursors are ring sideroblasts.

MDS with multilineage dysplasia (MDS-MLD) may include and/or be referred to as refractory cytopenia with multilineage dysplasia (RCMD). MDS-MLD typically involves two or three blood cell types being abnormal. In some embodiments, blood findings of MDS-MLD include one or more of: cytopenia(s), no or rare blasts (<1%), no Auer rods, and <1×10⁹/liter monocytes. In some embodiments, bone marrow findings of MDS-MLD include one or more of: dysplasia in ≥10% of cells in two or more myeloid lineages (e.g., neutrophil and/or erythroid precursors and/or megakaryocytes), <5% blasts, no Auer rods, and <15% ring sideroblasts.

MDS with ring sideroblasts (MDS-RS) may include and/or be referred to as refractory anemia with ring sideroblasts (RARS). MDS-RS typically involves a low number of one or more blood cell types. A characteristic feature of MDS-RS is that existing red blood cells in the bone marrow often contain a ring of excess iron called ring sideroblasts. Generally, at least 15% of sideroblasts are ring sideroblasts. In some embodiments, blood findings of MDS-RS include one or more of: anemia and no blasts. In some embodiments, bone marrow findings of MDS-RS include one or more of: erythroid dysplasia only and ≥15% of erythroid precursors are ring sideroblasts.

MDS with excess blasts (MDS-EB) has at least two types. In both type 1 (MDS-EB1) and type 2 (MDS-EB2), any of the three types of blood cells—red blood cells, white blood cells, or platelets—may be low and appear abnormal under a microscope. Very immature blood cells (blasts) are often found in the blood and bone marrow.

MDS-EB1 may include and/or be referred to as refractory anemia with excess blasts-1 (RAEB-1). In some embodiments, blood findings of MDS-EB1 include one or more of: cytopenia(s), <5% blasts, no Auer rods, and <1×10⁹/liter monocytes. In some embodiments, bone marrow findings of MDS-EB1 include one or more of: unilineage or multilineage dysplasia, blasts, and no Auer rods. In some embodiments, in MDS-EB1, blasts make up 5-9% of the cells in the bone marrow or 2-4% of the cells in the blood. In some embodiments, if the marrow myeloblast percentage is <5% but there are 2-4% myeloblasts in the blood, the MDS is classified as MDS-EB1; if, however, the marrow myeloblast percentage is <5% and there are 1% myeloblasts in the blood, the MDS is classified as MDS-U.

MDS-EB2 may include and/or be referred to as refractory anemia with excess blasts-2 (RAEB-2). In some embodiments, blood findings of MDS-EB2 include one or more of: cytopenia(s), 5-19% blasts, Auer rods, and <1×10⁹/liter monocytes. In some embodiments, bone marrow findings of MDS-EB2 include one or more of: unilineage or multilineage dysplasia, 10-19% blasts, and Auer rods. In some embodiments, in MDS-EB2, blasts make up 10-19% of the cells in the bone marrow and/or 5-19% of the cells in the blood. Cases with Auer rods and <5% myeloblasts in the blood and <10% in the marrow are generally classified as MDS-EB2.

MDS associated with isolated del(5q) typically involves low numbers of red blood cells, and cells having a specific mutation (e.g., a del (5q31-33) cytogenetic abnormality) in their DNA. In some embodiments, blood findings of MDS associated with isolated del(5q) include one or more of: anemia, usually normal or increased platelet count, and no or rare blasts (<1%). In some embodiments, blood findings of MDS associated with isolated del(5q) include one or more of: normal to increased megakaryocytes with hypolobated nuclei, <5% blasts, isolated del(5q) cytogenetic abnormality, and no Auer rods.

MDS-unclassified (MDS-U) typically involves reduced numbers of one of the three types of mature blood cells, and either the white blood cells or platelets look abnormal under a microscope. In some embodiments, blood findings of MDS-U include one or more of: cytopenias and ≤1% blasts. In some embodiments, bone marrow findings of MDS-U include one or more of: unequivocal dysplasia in less than 10% of cells in one or more myeloid cell lines <5% blasts.

A commonly used clinical prognostication tool for patients with MDS is the International Prognostic Scoring System (IPSS) (Greenberg et al. Blood. 1997;89:2079-2088). In this system, points are scored based on three criteria: the percentage of bone marrow blasts, the number of peripheral blood cytopenias, and the cytogenetic risk-class. Based on the total point score, the patient is assigned to 1 of 4 risk-categories that vary significantly in outcomes: low-risk (LR), intermediate-1 (INT-1), intermediate-2 (INT-2), and high-risk (HR). In some embodiments, an MDS patient is evaluated and/or classified according to IPSS criteria.

The Revised International Prognostic Scoring System (IPSS-R) is another exemplary standard for risk stratification and prognostication for patients with MDS (Greenberg et al. Blood. 2012;120(12):2454-2465). The IPSS-R distinguishes patients based on clinical characteristics and divides them into five defined risk groups: very low, low, intermediate, high, and very high. IPSS-R scores the disease based on marrow blast percentage, cytogenetics, hemoglobin levels, absolute neutrophil count (ANC), and platelet count. In some embodiments, an MDS patient is evaluated and/or classified according to IPSS-R criteria.

A patient may be referred to as having “higher-risk MDS” if the patient is classified as MDS of intermediate-2 risk or higher according to IPSS criteria or as MDS of high or very high risk according to IPSS-R criteria. In some embodiments, a patient having higher-risk MDS carries an SF3B1 mutation (e.g., an SF3B1 missense mutation) at a variant allele frequency of about 5% or higher. In some embodiments, a patient having higher-risk MDS is intolerant of hypomethylating agents (HMAs). In some embodiments, a patient having higher-risk MDS progresses and/or worsens in disease status after initiation of an HMA. In some embodiments, a patient having higher-risk MDS is unresponsive to about 4 treatment cycles of decitabine and/or about 6 treatment cycles of azacitidine.

A patient may be referred to as having “lower-risk MDS” if the patient is classified as MDS of intermediate-1 risk or lower according to IPSS criteria or as MDS of intermediate, low, or very low risk according to IPSS-R criteria. In some embodiments, a patient having lower-risk MDS carries an SF3B1 mutation (e.g., an SF3B1 missense mutation) at a variant allele frequency of about 5% or higher. In some embodiments, a patient having lower-risk MDS has an absolute neutrophil count (ANC) greater than or equal to about 500/μL (0.5×10⁹/L). In some embodiments, a patient having lower-risk MDS has a platelet count of less than about 50,000/μL/(50×10⁹/L).

In some embodiments, a patient having lower-risk MDS is transfusion-dependent for red blood cells (RBCs) and/or platelets. In some embodiments, a patient having lower-risk MDS is RBC transfusion-dependent according to International Working Group (IWG) 2006 Response Criteria for MDS (Cheson et al. Blood. 2006;108:419-425). In some embodiments, a lower-risk MDS patient who is RBC transfusion-dependent has received at least 4 U of RBCs within 8 weeks for hemoglobin (Hb) of <9 g/dL prior to the first dose of Compound 1. In some embodiments, a lower-risk patient who is RBC transfusion-dependent has failed erythropoiesis stimulating agents (ESA) (primary resistance or relapse after a response) and/or has serum erythropoietin (EPO) levels >500 U/L.

The term “transfusion dependence” or “transfusion-dependent,” as used herein, refers to a condition of severe anemia typically arising when erythropoiesis is reduced such that a patient continuously requires one or more transfusions (e.g., of red blood cells (RBCs), platelets, or both) over a specified interval (e.g., about 56 consecutive days (about 8 weeks)). A patient may be considered transfusion-dependent if the patient requires one or more transfusions over a period of at least 56 consecutive days. In some embodiments, a patient is transfusion-dependent prior to treatment with Compound 1. In some embodiments, the patient is transfusion-dependent for red blood cells (RBCs), platelets, or both. In some embodiments, the patient is transfusion-dependent for RBCs. In some embodiments, a transfusion-dependent patient has received at least 4 U of RBCs within 56 consecutive days (8 weeks) for hemoglobin (Hb) of <9 g/dL prior to the first dose of treatment with Compound 1. In some embodiments, a transfusion-dependent patient has been failed by erythropoiesis stimulating agents (ESAs). In some embodiments, a transfusion-dependent patient has a serum erythropoietin level >500 U/L. In some embodiments, a transfusion-dependent patient has platelet counts above 50×10⁹/L in the absence of transfusion for about 56 consecutive days (about 8 weeks).

The term “transfusion independence” or “transfusion-independent,” as used herein, refers to a condition in which a patient reduces the number or frequency of transfusions, or no longer requires transfusions, over a specified interval (e.g., about 56 consecutive days (about 8 weeks)). A patient may be considered transfusion-independent if the patient does not require or receive any transfusions for any period of at least 56 consecutive days during treatment with Compound 1 (e.g., Days 1 to 56, Days 2 to 57, Days 3 to 58, etc.). In some embodiments, the patient is transfusion-independent for at least 8 weeks, at least 9 weeks, at least 10 weeks, at least 12 weeks, at least 14 weeks, at least 16 weeks, or more. In some embodiments, the patient is transfusion-independent for red blood cells (RBCs), platelets, or both. In some embodiments, the patient is transfusion-independent for RBCs.

In some embodiments, transfusion independence is defined and/or evaluated as in, e.g., NCT00065156. In NCT00065156 (“Lenalidomide Safety/Efficacy in Myelodysplastic Syndromes (MDS) Associated With a Deletion (Del)(5q) Cytogenetic Abnormality”), transfusion independence was defined as a period of at least 56 consecutive days in which no transfusions were given to a patient and the patient's hemoglobin concentration rose by at least 1 g per deciliter; minor response was defined as a reduction of at least 50% in the number of transfusions as compared with baseline requirements; and the rise in the hemoglobin concentration in patients who no longer required transfusions was calculated as the difference between the maximum hemoglobin concentration and the minimum pre-transfusion value during the 56 days (8 weeks) prior to treatment.

### Therapeutic Methods and Uses

In some embodiments, the present disclosure provides methods and uses for novel biomarkers to treat transfusion dependence in a patient with MDS. More specifically, in some embodiments, the present disclosure provides a method of treating transfusion dependence in a patient with MDS, comprising administering a therapeutically effective amount of Compound 1 to the transfusion-dependent MDS patient who has an elevated TMEM14C AJ/CJ ratio. In some embodiments, the present disclosure provides a method of treating transfusion dependence in a patient with MDS, comprising administering a therapeutically effective amount of Compound 1 to the transfusion-dependent MDS patient who has an elevated level of aberrant junction TMEM14C transcripts (TMEM14C AJ) as compared to a control (e.g., a control subject who does not have MDS). In some embodiments, the present disclosure provides use of a TMEM14C AJ/CJ ratio as a biomarker for treating transfusion dependence in a patient with MDS. In some embodiments, the present disclosure provides use of a TMEM14C AJ/CJ ratio as a biomarker in the manufacture of a medicament for treating transfusion dependence in a patient with MDS. In some embodiments, the present disclosure provides a TMEM14C AJ/CJ ratio for use as a biomarker for treating transfusion dependence in a patient with MDS. In some embodiments, the treating comprises administering a therapeutically effective amount of Compound 1 to the transfusion-dependent MDS patient who has an elevated TMEM14C AJ/CJ ratio. In some embodiments, the patient or a biological sample from the patient has a low level of TMEM14C expression. In some embodiments, Compound 1 reduces or inhibits TMEM14C aberrant splicing in the patient.

In some embodiments, the present disclosure provides a method of treating transfusion dependence in a patient with MDS, comprising: (a) determining that the transfusion-dependent MDS patient has an elevated TMEM14C AJ/CJ ratio; and (b) administering a therapeutically effective amount of Compound 1 to the patient. In some embodiments, the present disclosure provides a method of treating transfusion dependence in a patient with MDS, comprising: (a) determining that the transfusion-dependent MDS patient has an elevated level of aberrant junction TMEM14C transcripts (TMEM14C AJ) as compared to a control (e.g., a control subject who does not have MDS); and (b) administering a therapeutically effective amount of Compound 1 to the patient. In some embodiments, the present disclosure provides a method of treating transfusion dependence in a patient with MDS, comprising: (a) selecting a transfusion-dependent MDS patient for treatment by determining that the patient has an elevated TMEM14C AJ/CJ ratio; and (b) administering a therapeutically effective amount of Compound 1 to the patient. In some embodiments, the present disclosure provides use of a TMEM14C AJ/CJ ratio as a biomarker for treating transfusion dependence in a patient with MDS. In some embodiments, the present disclosure provides use of a TMEM14C AJ/CJ ratio as a biomarker in the manufacture of a medicament for treating transfusion dependence in a patient with MDS. In some embodiments, the present disclosure provides a TMEM14C AJ/CJ ratio for use as a biomarker for treating transfusion dependence in a patient with MDS. In some embodiments, the treating comprises: (a) determining that the transfusion-dependent MDS patient has an elevated TMEM14C AJ/CJ ratio; and (b) administering a therapeutically effective amount of Compound 1 to the patient. In some embodiments, the patient or a biological sample from the patient has a low level of TMEM14C expression. In some embodiments, Compound 1 reduces or inhibits TMEM14C aberrant splicing in the patient.

In some embodiments, the present disclosure also provides methods of identifying MDS patients suitable for treatment with Compound 1 and/or predicting or monitoring treatment efficacy in an MDS patient. In some embodiments, the present disclosure provides a method of identifying a transfusion-dependent MDS patient suitable for treatment with Compound 1, comprising: (a) determining that the patient has an elevated TMEM14C AJ/CJ ratio; and (b) identifying the patient as suitable for treatment with Compound 1. In some embodiments, the present disclosure provides use of a TMEM14C AJ/CJ ratio as a biomarker for identifying a transfusion-dependent MDS patient suitable for treatment with Compound 1. In some embodiments, the present disclosure provides use of a TMEM14C AJ/CJ ratio as a biomarker in the manufacture of a composition for identifying a transfusion-dependent MDS patient suitable for treatment with Compound 1. In some embodiments, the present disclosure provides a TMEM14C AJ/CJ ratio for use as a biomarker for identifying a transfusion-dependent MDS patient suitable for treatment with Compound 1. In some embodiments, the identifying comprises: (a) determining that the patient has an elevated TMEM14C AJ/CJ ratio; and (b) identifying the patient as suitable for treatment with Compound 1. In some embodiments, the patient or a biological sample from the patient has a low level of TMEM14C expression. In some embodiments, Compound 1 reduces or inhibits TMEM14C aberrant splicing in the patient.

In some embodiments, the present disclosure provides a method of monitoring treatment efficacy in a transfusion-dependent MDS patient, comprising: (a) determining that the patient has an elevated TMEM14C AJ/CJ ratio; (b) administering a therapeutically effective amount of Compound 1 to the patient; and (c) determining the TMEM14C AJ/CJ ratio in the patient after administration, wherein a reduction in the TMEM14C AJ/CJ ratio after administration indicates an effective treatment. In some embodiments, the TMEM14C AJ/CJ ratio remains elevated after step (c), and the method further comprises administering an additional dose of Compound 1 to the patient. In some embodiments, the method further comprises administering additional doses of Compound 1 to the patient until the TMEM14C AJ/CJ ratio is no longer elevated. In some embodiments, the present disclosure provides use of a TMEM14C AJ/CJ ratio as a biomarker for monitoring treatment efficacy in a transfusion- dependent MDS patient. In some embodiments, the present disclosure provides use of a TMEM14C AJ/CJ ratio as a biomarker in the manufacture of a composition for monitoring treatment efficacy in a transfusion-dependent MDS patient. In some embodiments, the present disclosure provides a TMEM14C AJ/CJ ratio for use as a biomarker for monitoring treatment efficacy in a transfusion-dependent MDS patient. In some embodiments, the monitoring comprises: (a) determining that the patient has an elevated TMEM14C AJ/CJ ratio; (b) administering a therapeutically effective amount of Compound 1 to the patient; and (c) determining the TMEM14C AJ/CJ ratio in the patient after administration, wherein a reduction in the TMEM14C AJ/CJ ratio after administration indicates an effective treatment. In some embodiments, the TMEM14C AJ/CJ ratio remains elevated after step (c), and the monitoring further comprises administering an additional dose of Compound 1 to the patient. In some embodiments, the monitoring further comprises administering additional doses of Compound 1 to the patient until the TMEM14C AJ/CJ ratio is no longer elevated. In some embodiments, the patient or a biological sample from the patient has a low level of TMEM14C expression. In some embodiments, Compound 1 reduces or inhibits TMEM14C aberrant splicing in the patient.

In some embodiments of the methods and uses disclosed herein, an MDS patient is a patient who has been diagnosed with MDS according to the WHO 2008 classification (reviewed in Vardiman et al. Blood 2009;114(5):937-951). In some embodiments, the MDS is MDS with multilineage dysplasia (MDS-MLD), MDS with single lineage dysplasia (MDS-SLD), MDS with ring sideroblasts (MDS-RS), MDS with excess blasts (MDS-EB), MDS associated with isolated del(5q), or MDS-unclassified (MDS-U). In some embodiments, the MDS is MDS of intermediate-1 risk or lower according to the International Prognostic Scoring System. In some embodiments, the MDS is MDS of intermediate-2 risk or higher according to the International Prognostic Scoring System. In some embodiments, the MDS is MDS-MLD. In some embodiments, the MDS is MDS-EB. In some embodiments, the MDS-EB is MDS-EB1 or MDS-EB2. In some embodiments, the MDS is MDS-EB2.

In some embodiments, the MDS is lower-risk MDS, i.e., intermediate-1 risk or lower according to IPSS criteria. In some embodiments, a patient having lower-risk MDS carries an SF3B1 mutation (e.g., an SF3B1 missense mutation) at a variant allele frequency of about 5% or higher. In some embodiments, a patient having lower-risk MDS has an absolute neutrophil count (ANC) greater than or equal to about 500/μL (0.5×10⁹/L). In some embodiments, a patient having lower-risk MDS has a platelet count of less than about 50,000/μL (50×10⁹/L).

In some embodiments, a patient having lower-risk MDS is transfusion-dependent for red blood cells (RBCs) and/or platelets. In some embodiments, a patient having lower-risk MDS is RBC transfusion-dependent according to IWG 2006 Response Criteria for MDS (Cheson et al. Blood. 2006;108:419-425). In some embodiments, a lower-risk MDS patient who is RBC transfusion-dependent has received at least about 4 U of RBCs (e.g., 4 U, 6 U, 8 U, 10 U, or more of RBCs) within about 6 to about 10 weeks (e.g., within about 8 weeks) prior to the first dose of Compound 1. In some embodiments, the at least about 4 U of RBCs are for hemoglobin (Hb) of less than about 9 g/dL (e.g., 9 g/dL, 8 g/dL, 7 g/dL, 6 g/dL, or less). In some embodiments, a lower-risk MDS patient who is RBC transfusion-dependent has received at least 4 U of RBCs within 8 weeks for hemoglobin (Hb) of <9 g/dL prior to the first dose of Compound 1. In some embodiments, a lower-risk patient who is RBC transfusion-dependent has failed erythropoiesis stimulating agents (ESA) (primary resistance or relapse after a response) and/or has serum EPO levels >500 U/L.

In some embodiments, the diagnosis of MDS is made or confirmed using a physical examination and/or one or more diagnostic tests. Exemplary tests used to diagnose MDS include blood tests, peripheral (circulating) blood smears, bone marrow aspiration and biopsy, molecular testing, cytogenetic (chromosomal) analysis, and immunophenotyping.

In some embodiments, an MDS patient has been diagnosed with MDS using a blood test, alone or in combination with one or more additional diagnostic tests. A complete blood count (CBC) can measure the numbers of red blood cells, white blood cells, and platelets. Blood tests may also be done to rule out other conditions that can cause symptoms similar to MDS, such as low levels of vitamin B12, folate, copper, and thyroid problems.

In some embodiments, an MDS patient has been diagnosed with MDS using a peripheral (circulating) blood smear, alone or in combination with one or more additional diagnostic tests. In some embodiments, a drop of blood is placed on a slide, smeared into a thin film, placed under a microscope for examination, and the number and/or percentages of the different cell types are counted. The appearance of cells under the microscope (i.e., cell morphology) may also be observed to identify if or how the cells differ from healthy cells.

In some embodiments, an MDS patient has been diagnosed with MDS using bone marrow aspiration and/or biopsy, alone or in combination with one or more additional diagnostic tests. These two procedures are similar and are often done at the same time to examine the bone marrow. Bone marrow has both a solid and liquid part. A bone marrow aspiration removes a sample of the fluid with a needle. A bone marrow biopsy is the removal of a small amount of solid tissue using a needle. In some embodiments, the sample(s) are then analyzed to determine the percentage of red blood cells, white blood cells, platelets, and/or blasts. In general, the appearance of the bone marrow tissue, along with blood cell counts and chromosomal analysis, may be used to confirm a diagnosis of MDS.

In some embodiments, an MDS patient has been diagnosed with MDS using molecular testing, alone or in combination with one or more additional diagnostic tests. Laboratory tests (e.g., on a bone marrow sample) may be performed to identify specific genes, proteins, and/or other factors unique to MDS.

In some embodiments, an MDS patient has been diagnosed with MDS using cytogenetic (chromosomal) analysis, alone or in combination with one or more additional diagnostic tests. The chromosomes of the cells in the blood and/or bone marrow may show specific abnormalities that help to identify MDS and differentiate MDS from other blood disorders.

In some embodiments, an MDS patient has been diagnosed with MDS using immunophenotyping, alone or in combination with one or more additional diagnostic tests. Immunophenotyping is the examination of antigens, a specific type of protein, on the surface of the cells. Immunophenotyping can help to identify the type of MDS.

In various embodiments of the methods and uses disclosed herein, an MDS patient that has been diagnosed with MDS is further evaluated for the presence or absence of one or more mutations in a spliceosome protein, e.g., one or more mutations in SF3B1, SRSF2, U2AF1, and/or ZRSR2 (e.g., one or more mutations in SF3B1). In some embodiments, the patient or a biological sample from the patient comprises a mutation in one or more genes associated with RNA splicing. In some embodiments, the patient or a biological sample from the patient comprises a mutation in one or more genes selected from SF3E31, SRSF2, U2AF1, and ZRSR2.

In some embodiments, the patient or a biological sample from the patient (e.g., a blood sample, a bone marrow sample, and/or a urine sample) comprises a mutation in SF3E31. In some embodiments, the mutation in SF3B1 comprises or consists of a mutation at one or more of positions E622, H662, K666, K700, R625, or V701 in SF3B1. In some embodiments, the mutation in SF3B1 comprises or consists of a mutation at one or more of positions H662, K700, or R625 in SF3B1. In some embodiments, the mutation in SF3B1 comprises or consists of a mutation at position K700 in SF3B1. In some embodiments, the mutation at position K700 is K700E. In some embodiments, the mutation at position R625 is R625C. In some embodiments, the mutation in SF3B1 comprises or consists of K700E, R625C, and/or at least one additional mutation (e.g., at least one other HEAT domain mutation) in SF3B1.

In some embodiments, the patient or a biological sample from the patient (e.g., a blood sample, a bone marrow sample, and/or a urine sample) comprises a mutation in SRSF2. In some embodiments, a mutation in SRSF2 comprises or consists of P95H, P95L, P95_R102 del, and/or at least one additional mutation in SRSF2.

In some embodiments, the patient or a biological sample from the patient (e.g., a blood sample, a bone marrow sample, and/or a urine sample) comprises a mutation in U2AF1. In some embodiments, the mutation in U2AF1 comprises or consists of Q157P, S34F, and/or at least one additional mutation (e.g., at least one other hotspot mutation) in U2AF1.

In some embodiments, the patient or a biological sample from the patient (e.g., a blood sample, a bone marrow sample, and/or a urine sample) comprises a mutation in ZRSR2. In some embodiments, the mutation in ZRSR2 comprises or consists of at least one truncating or nonsense mutation in ZRSR2.

Exemplary methods for detecting mutations in spliceosome proteins, such as those identified above, are described herein.

In various embodiments of the methods and uses disclosed herein, determining the TMEM14C AJ/CJ ratio (e.g., an elevated TMEM14C AJ/CJ ratio) in a patient with MDS comprises obtaining a biological sample from the patient and determining the TMEM14C AJ/CJ ratio in the sample. In some embodiments, the biological sample comprises a blood sample. In some embodiments, the biological sample comprises a bone marrow sample. In some embodiments, the biological sample comprises a urine sample.

Samples can be obtained from a variety of biological sources. Exemplary biological samples include but are not limited to blood or a blood fraction, plasma, saliva, serum, sputum, urine, cerebral spinal fluid, one or more cells, a cell culture, a cell line, a cellular extract, an organ, an organelle, a tissue sample, a tissue biopsy, a skin sample, a bone marrow sample, a stool sample, and the like. Blood samples may be whole blood, partially purified blood, and/or a fraction of whole or partially purified blood, such as peripheral blood mononucleated cells (PBMCs) or plasma. Bone marrow samples may be bone marrow aspirates and/or bone marrow biopsies. Samples may be obtained directly from a patient or derived from cells obtained from a patient, such as cultured cells derived from a biological fluid or tissue sample. Samples may also be archived samples, such as cryopreserved samples.

Biological samples may be used in any of the methods or uses disclosed herein. In some embodiments, a biological sample is obtained from a patient having or suspected of having MDS, e.g., one diagnosed with MDS and confirmed as having an SF3B1 mutation. In some embodiments, the biological sample comprises a blood sample or a bone marrow sample. In some embodiments, the blood sample comprises peripheral blood or plasma. In some embodiments, the bone marrow sample comprises a bone marrow aspirate or a bone marrow biopsy. In some embodiments, the biological sample comprises a urine sample.

In some embodiments of the methods and uses disclosed herein, the patient or a biological sample from the patient comprises an elevated TMEM14C AJ/CJ ratio, i.e., a TMEM14C AJ/CJ ratio greater than about 0.1 (e.g., greater than about 0.1, about 0.2, about 0.5, about 1, about 2, about 4, about 10, about 15, about 20, or about 30), e.g., as measured by nucleic acid barcoding. In some embodiments, an elevated TMEM14C AJ/CJ ratio is greater than about 0.1, 0.2, 0.3, 0.4, 0.5, 0.6, 0.7, 0.8, 0.9, or more, as measured by nucleic acid barcoding. In some embodiments, an elevated TMEM14C AJ/CJ ratio is greater than about 1, 2, 3, 4, 5, 6, 7, 8, 9, or more, as measured by nucleic acid barcoding. In some embodiments, an elevated TMEM14C AJ/CJ ratio is greater than about 10, 11, 12, 13, 14, 15, 16, 17, 18, 19, or more, as measured by nucleic acid barcoding. In some embodiments, an elevated TMEM14C AJ/CJ ratio is greater than about 20, 25, 30, 35, or more (e.g., about 40, 45, 50, or more), as measured by nucleic acid barcoding. In some embodiments, an elevated TMEM14C AJ/CJ ratio is a ratio greater than about 4, as measured by nucleic acid barcoding.

### Detection of Splice Variants

Certain embodiments of the methods and uses described herein involve detection and/or quantification of splice variants. A variety of methods exist for detecting and quantifying nucleic acids, and each may be adapted for detection of splice variants in the described embodiments. Exemplary methods include an assay to quantify nucleic acids such as nucleic acid barcoding, nanoparticle probes, in situ hybridization, microarray, nucleic acid sequencing, and PCR-based methods, including real-time PCR (RT-PCR).

In some embodiments of the methods and uses disclosed herein, the TMEM14C AJ/CJ ratio is determined by measuring RNA transcripts in the patient or in a biological sample from the patient. In some embodiments, measuring RNA transcripts comprises nucleic acid barcoding and/or RT-PCR. In some embodiments, measuring RNA transcripts comprises nucleic acid barcoding.

Nucleic acid assays utilizing barcoding technology such as NanoString® assays (NanoString Technologies) may be performed, for example, as described in U.S. Pat. Nos. 8,519,115; 7,919,237; and in Kulkarni (Current Protocols in Molecular Biology, 2011;94:25B.10.1-25B.10.17). In an exemplary assay, a pair of probes is used to detect a particular nucleotide sequence of interest, such as a particular splice variant of interest. The probe pair consists of a capture probe and a reporter probe that each include a sequence of from about 35 to 50 bases in length that is specific for a target sequence. The capture probe includes an affinity label such as biotin at its 3′ end that provides a molecular handle for surface-attachment of target mRNAs for digital detection, and the reporter probe includes a unique color code at its 5′ end that provides molecular barcoding of the hybridized mRNA target sequence. Capture and reporter probe pairs are hybridized to target mRNA in solution, and after excess probes are removed, the target mRNA-probe complexes are immobilized in an nCounter® cartridge. A digital analyzer acquires direct images of the surface of the cartridge to detect color codes corresponding to specific mRNA splice variant sequences. The number of times a color-coded barcode for a particular splice variant is detected reflects the levels of a particular splice variant in the mRNA library. For the detection of splice variants, either the capture or the reporter probe may span a given splice variant's exon-exon or intron-exon junction. In other embodiments, one or both of the capture and reporter probes' target sequences correspond to the terminal sequences of two exons at an exon-exon junction or to the terminal sequences of an intron and an exon at an intron-exon junction, whereby one probe extends to the exon-exon or intron-exon junction, but does not span the junction, and the other probe binds a sequence that begins on opposite side of the junction and extends into the respective exon or intron.

In exemplary PCR-based methods, a particular splice variant may be detected by specifically amplifying a sequence that contains the splice variant. For example, the method may employ a first primer specifically designed to hybridize to a first portion of the splice variant, where the splice variant is a sequence that spans an exon-exon or intron-exon junction at which alternative splicing occurs. The method may further employ a second opposing primer that hybridizes to a segment of the PCR extension product of the first primer that corresponds to another sequence in the gene, such as a sequence at an upstream or downstream location. The PCR detection method may be quantitative (or real-time) PCR. In some embodiments of quantitative PCR, an amplified PCR product is detected using a nucleic acid probe, wherein the probe may contain one or more detectable labels. In certain quantitative PCR methods, the amount of a splice variant of interest is determined by detecting and comparing levels of the splice variant to an appropriate internal control.

Exemplary methods for detecting splice variants using an in situ hybridization assay such as RNAscope® (Advanced Cell Diagnostics) include those described by Wang et al. (J Mol Diagn. 2012;14(1):22-29). RNAscope® assays may be used to detect splice variants by designing a pair of probes that targets a given splice variant and hybridizing the probes to target RNA in fixed and permeabilized cells. Target probes are designed to hybridize as pairs which, when hybridized to the target sequence, create a binding site for a preamplifier nucleic acid. The preamplifier nucleic acid, in turn, harbors multiple binding sites for amplifier nucleic acids, which in turn contain multiple binding sites for a labeled probe carrying a chromogenic or fluorescent molecule. In some embodiments, one of the RNAscope® target probes spans a given splice variant's exon-exon or intron-exon junction. In other embodiments, the target probes' target sequences correspond to the terminal sequences of two exons at an exon-exon junction or to the terminal sequences of an intron and an exon at an intron-exon junction, whereby one probe in the target probe pair extends to the exon-exon or intron-exon junction, but does not span the junction, and the other probe binds a sequence beginning on opposite side of the junction and extending into the respective exon or intron.

Exemplary methods for detecting splice variants using nanoparticle probes such as SmartFlare™ (Millipore) include those described in Seferos et al. (J Am Chem Soc. 2007;129(50):15477-15479) and Prigodich et al. (Anal. Chem. 2012;84(4):2062-2066). SmartFlare™ detection probes may be used to detect splice variants by generating gold nanoparticles that are modified with one or more nucleic acids that include nucleotide recognition sequences that (1) are each complementary to a particular splice variant to be detected and (2) are each hybridized to a complementary fluorophore-labeled reporter nucleic acid. Upon uptake of the probe by a cell, a target splice variant sequence may hybridize to the one or more nucleotide recognition sequences and displace the fluorophore-labeled reporter nucleic acid. The fluorophore-labeled reporter nucleic acid, whose fluorophore had been quenched due to proximity to the gold nanoparticle surface, is then liberated from the gold nanoparticle, and the fluorophore may then be detected when free of the quenching effect of the nanoparticle. In some embodiments, nucleotide recognition sequences in the probes recognize a sequence that spans a given splice variant's exon-exon or intron-exon junction. In some embodiments, nucleotide recognition sequences in the probes recognize a sequence that is only on one side of the splice variant's exon-exon or intron-exon junction, including a sequence that terminates at the junction and a sequence that terminates one or more nucleotides away from the junction.

Exemplary methods for detecting splice variants using nucleic acid sequencing include RNA sequencing (RNA-Seq) described in Ren et al. (Cell Res. 2012;22:806-821); and van Dijk et al. (Trends Genet. 2014;30(9):418-426). In some embodiments, high-throughput sequencing, such as next-generation sequencing (NGS) technologies, may be used to detect splice variants. For example, the method may employ commercial sequencing platforms available for RNA-Seq, such as, e.g., Illumina, SOLID, Ion Torrent, and Roche 454. In some embodiments, the sequencing method may include pyrosequencing. For example, a sample may be mixed with sequencing enzymes and primer and exposed to a flow of one unlabeled nucleotide at a time, allowing synthesis of the complementary DNA strand. When a nucleotide is incorporated, pyrophosphate is released leading to light emission, which is monitored in real time. In some embodiments, the sequencing method may include semiconductor sequencing. For example, proton instead of pyrophosphate may be released during nucleotide incorporation and detected in real time by ion sensors. In some embodiments, the method may include sequencing with reversible terminators. For example, the synthesis reagents may include primers, DNA polymerase, and four differently labelled, reversible terminator nucleotides. After incorporation of a nucleotide, which is identified by its color, the 3′ terminator on the base and the fluorophore are removed, and the cycle is repeated. In some embodiments, the method may include sequencing by ligation. For example, a sequencing primer may be hybridized to an adapter, with the 5′ end of the primer available for ligation to an oligonucleotide hybridizing to the adjacent sequence. A mixture of octamers, in which bases 4 and 5 are encoded by one of four color labels, may compete for ligation to the primer. After color detection, the ligated octamer may be cleaved between position 5 and 6 to remove the label, and the cycle may be repeated. Thereby, in the first round, the process may determine possible identities of bases in positions 4, 5, 9, 10, 14, 15, etc. The process may be repeated, offset by one base using a shorter sequencing primer, to determine positions 3, 4, 8, 9, 13, 14, etc., until the first base in the sequencing primer is reached.

Other nucleic acid detection and analytical methods that also distinguish between splice variants of a given exon-exon or intron-exon junction in a gene by identifying the nucleotide sequence on both sides of the junction may be utilized to detect or quantify splice variants. For example, splice variants of an exon-exon junction may be detected by primer extension methods in which a primer that binds to one exon is extended into the exon on the other side of the junction according to the sequence of that adjacent exon. See, e.g., McCullough et al. (Nucleic Acids Research, 2005;33(11):e99); and Milani et al. (Clin. Chem. 2006;52:202-211). Detection of variants on a large scale may be performed using expression microarrays that carry exon-exon or intron-exon junction probes, as described, for example, in Johnson et al. (Science 2003;302:2141-2144); and Modrek et al. (Nucleic Acids Res. 2001;29: 2850-2859).

Various embodiments include reagents for detecting splice variants of TMEM14C. In one example, reagents include NanoString® probes designed to measure the amount of one or more aberrant or canonical splice variants of TMEM14C. Probes for nucleic acid quantification assays such as barcoding (e.g., NanoString®), nanoparticle probes (e.g., SmartFlare™), in situ hybridization (e.g., RNAscope®), microarray, nucleic acid sequencing, and PCR-based assays may be designed as set forth above.

In these exemplary methods or in other methods for nucleic acid detection, aberrant splice variants may be identified using probes, primers, or other reagents which specifically recognize the nucleic acid sequence that is present in the aberrant splice variant but absent in the canonical splice variant. In other embodiments, the aberrant splice variant is identified by detecting the sequence that is specific to the aberrant splice variant in the context of the junction in which it occurs, i.e., the unique sequence is flanked by the sequences which are present on either side of the splice junction in the canonical splice variant. In such cases, the portion of the probe, primer, or other detection reagent that specifically recognizes its target sequence may have a length that corresponds to the length of the aberrant sequence or to or a portion of the aberrant sequence. In other embodiments, the portion of the probe, primer, or other detection reagent that specifically recognizes its target sequence may have a length that corresponds to the length of the aberrant sequence plus the length of a chosen number of nucleotides from one or both of the sequences which flank the aberrant sequence at the splice junction. Generally, the probe or primer should be designed with a sufficient length to reduce non-specific binding. Probes, primers, and other reagents that detect aberrant or canonical splice variants may be designed according to the technical features and formats of a variety of methods for detection of nucleic acids.

### Therapeutic Compounds

In various embodiments of the disclosure, the therapeutic compound used in the disclosed methods and uses is a splicing modulator such as Compound 1, or an alternate agent identified with treatment of the same patient population (e.g., transfusion-dependent MDS patients). In some embodiments, the therapeutic compound is a splicing modulator or an alternate agent such as a TGFβ1 modulator. An exemplary TGFβ1 modulator that has been evaluated in transfusion-dependent MDS patients is luspatercept. Luspatercept, a recombinant fusion protein that binds TGFβ superfamily ligands, is described, for example, in Fenaux et al. (N Engl J Med. 2020;382(2):140-151), which is incorporated herein by reference.

In various embodiments, the therapeutic compound is a splicing modulator. In some embodiments, the splicing modulator is a modulator of the SF3b spliceosome complex. Such modulators may be natural compounds or synthetic compounds. Non-limiting examples of splicing modulators and categories of such modulators include pladienolide (e.g., pladienolide B or pladienolide D), pladienolide derivatives (e.g., pladienolide B or pladienolide D derivatives), herboxidiene, herboxidiene derivatives, spliceostatin, spliceostatin derivatives, sudemycin, or sudemycin derivatives. As used herein, the terms “derivative” and “analog” when referring to a splicing modulator, or the like, means any such compound that retains essentially the same, similar, or enhanced biological function or activity as the original compound but has an altered chemical or biological structure.

In various embodiments, the splicing modulator comprises an SF3B1 modulator. A variety of SF3B1 modulating compounds are known in the art and can be used in the methods and uses described herein. Exemplary SF3B1 modulators include but are not limited to Compound 1, pladienolides (e.g., pladienolide B, pladienolide D), pladienolide derivatives (e.g., E7107 (Compound 45 of WO 2003/099813)), aryl pladienolides, aryl pladienolide derivatives, herboxidienes, and herboxidiene derivatives. Non-limiting examples of SF3B1 modulating compounds are disclosed in U.S. Pat. No. 9,481,669 B2, International Application No. PCT/US2016/062525 (Intl. Pub. No. WO 2017/087667), International Application No. PCT/US2019/026313 (Intl. Pub. No. WO 2019/199667), International Application No. PCT/US2019/026992 (Intl. Pub. No. WO 2019/200100), International Application No. PCT/US2019/066029 (Intl. Pub. No. WO 2020/123836), and International Application No. PCT/US2019/035015 (Intl. Pub. No. WO 2019/232449), all of which are incorporated herein by reference for the disclosure and/or synthesis of such compounds.

In some embodiments, the splicing modulator, e.g., an SF3B1 modulator, is any one or more of the exemplary SF3B1 modulating compounds described or incorporated by reference herein. For instance, in various embodiments, the SF3B1 modulator is Compound 1. In other embodiments, the SF3B1 modulator is pladienolide B, pladienolide D, or E7107. In other embodiments, the SF3B1 modulator is any one or more of the SF3B1 modulating compounds disclosed in U.S. Pat. No. 9,481,669 B2. In other embodiments, the SF3B1 modulator is any one or more of the SF3B1 modulating compounds disclosed in International Application No. PCT/US2016/062525 (Intl. Pub. No. WO 2017/087667). In other embodiments, the SF3B1 modulator is any one or more of the SF3B1 modulating compounds disclosed in International Application No. PCT/US2019/026313 (Intl. Pub. No. WO 2019/199667). In other embodiments, the SF3B1 modulator is any one or more of the SF3B1 modulating compounds disclosed in International Application No. PCT/US2019/026992 (Intl. Pub. No. WO 2019/200100). In other embodiments, the SF3B1 modulator is any one or more of the SF3B1 modulating compounds disclosed in International Application No. PCT/US2019/066029 (Intl. Pub. No. WO 2020/123836). In other embodiments, the SF3B1 modulator is any one or more of the SF3B1 modulating compounds disclosed in International Application No. PCT/US2019/035015 (Intl. Pub. No. WO 2019/232449). All patents and publications recited in this paragraph are incorporated by reference in their entirety and, in particular, for the splicing modulators (e.g., SF3B1 modulators) disclosed therein.

In some embodiments, the splicing modulator and/or SF3B1 modulator (e.g., any one or more of the exemplary SF3B1 modulating compounds described or incorporated by reference herein) modulates and/or inhibits SF3B1. In some embodiments, the splicing modulator and/or SF3B1 modulator (e.g., any one or more of the exemplary SF3B1 modulating compounds described or incorporated by reference herein) is an SF3B1 inhibitor.

In some embodiments, the splicing modulator and/or SF3B1 modulator is a pladienolide or pladienolide derivative. As used herein, a “pladienolide derivative” refers to a compound which is structurally related to a member of the family of natural products known as the pladienolides and which retains one or more biological functions of the starting compound. Pladienolides were first identified in the bacteria Streptomyces platensis (Mizui et al. J Antibiot. 2004;57:188-196) as being potently cytotoxic and resulting in cell cycle arrest in the G1 and G2/M phases of the cell cycle (e.g., Bonnal et al. Nat Rev Drug Dis. 2012;11:847-859). There are seven naturally occurring pladienolides, pladienolide A-G (Mizui et al. J Antibiot. 2004;57:188-196; Sakai et al. J Antibiot. 2004;57:180-187). One of these compounds, pladienolide B, targets the SF3b spliceosome to inhibit splicing and alter the pattern of gene expression (Kotake et al. Nature Chemical Biology 2007;3:570-575). Certain pladienolide B derivatives are described in WO 2002/060890; WO 2004/011459; WO 2004/011661; WO 2004/050890; WO 2005/052152; WO 2006/009276; and WO 2008/126918, each of which is incorporated herein by reference.

U.S. Pat. Nos. 7,884,128 and 7,816,401 describe exemplary methods of synthesizing pladienolide B and D and are each incorporated herein by reference for such methods. Synthesis of pladienolide B and D may also be performed using the exemplary methods described in Kanada et al. (Angew Chem Int Ed. 2007;46:4350-4355). Kanada et al. and Intl. Pub. No. WO 2003/099813 describe exemplary methods for synthesizing E7107 (Compound 45 of WO 2003/099813) from pladienolide D (11107D of WO 2003/099813). A corresponding U.S. Pat. No. is 7,550,503 to Kotake et al. Each of these references is incorporated herein for the described synthesis methods. In some embodiments, the SF3B1 modulator is pladienolide B, pladienolide D, or E7107. In some embodiments, the SF3B1 modulator is Compound 1.

In various embodiments, the splicing modulator and/or SF3B1 modulator is Compound 1, i.e., at least one entity chosen from the compound of Formula I and pharmaceutically acceptable salts thereof. Formula I may be represented by the following:

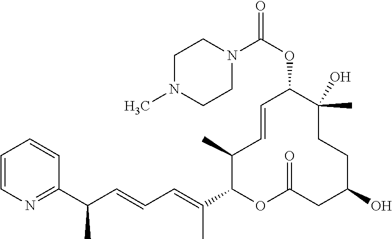 [SMILES: C/C(=C\C=C\[C@@H](C)c1ccccn1)[C@H]1OC(=O)C[C@H](O)CC[C@@](C)(O)[C@@H](OC(=O)N2CCN(C)CC2)/C=C/[C@@H]1C]

and/or the chemical name (2S,3S,4E,6S,7R,10R)-7,10-dihydroxy-3,7-dimethyl-12-oxo-2-[(2E,4E,6R)-6-(pyridine-2-yl)hepta-2,4-dien-2-yl]oxacyclododec-4-en-6-yl-4-methylpiperazine-1-carboxylate. Synthesis of Compound 1 is described in U.S. Pat. No. 9,481,669 B2 and International Application No. PCT/US2016/062525 (Intl. Pub. No. WO 2017/087667), all of which are incorporated herein by reference.

### Treatment Regimens

Various embodiments of the disclosure include administration of Compound 1. In some embodiments, the methods and uses disclosed herein comprise administering a therapeutically effective amount of Compound 1 to a patient. In some embodiments, Compound 1 reduces or inhibits TMEM14C aberrant splicing in the patient. In some embodiments, the patient is a transfusion-dependent MDS patient who has an elevated ratio of aberrant junction to canonical junction TMEM14C transcripts (TMEM14C AJ/CJ ratio). In some embodiments, the patient is a transfusion-dependent MDS patient who has an elevated TMEM14C AJ/CJ ratio and one or more mutations in a spliceosome protein, e.g., one or more mutations in SF3E31. In some embodiments, the patient is a transfusion-dependent MDS patient who has an elevated TMEM14C AJ/CJ ratio and a low level of TMEM14C expression. In some embodiments, the patient is a transfusion-dependent MDS patient who has an elevated TMEM14C AJ/CJ ratio, a low level of TMEM14C expression, and one or more mutations in a spliceosome protein, e.g., one or more mutations in SF3E31. Exemplary SF3B1 mutations include mutations at one or more of positions E622, H662, K666, K700, R625, or V701 in SF3B1. In some embodiments, a mutation in SF3B1 comprises or consists of a mutation at one or more of positions H662, K700, or R625 in SF3B1. In some embodiments, a mutation in SF3B1 comprises or consists of a mutation at position K700 in SF3B1. In some embodiments, the mutation at position K700 is K700E. In some embodiments, the mutation at position R625 is R625C. In some embodiments, a mutation in SF3B1 comprises K700E and/or R625C. Additional non-limiting examples of SF3B1 mutations are disclosed in U.S. Pat. No. 10,889,866 B2, and International Application No. PCT/US2016/049490 (Intl. Pub. No. WO 2017/040526), each of which is incorporated herein by reference for the disclosure of such mutations.

One of ordinary skill will understand that a specific dosage and treatment regimen for any particular patient will depend upon a variety of factors, including the activity of the specific compound employed, the age, body weight, general health, sex, diet, time of administration, rate of excretion, drug combination, the judgment of the treating physician, and the severity of the particular disease being treated. In some embodiments, Compound 1 used in the methods and uses described herein may be administered initially in a suitable dosage that may be adjusted as required, depending on the clinical response. In general, a suitable dose of Compound 1 may be an amount of the compound that is the lowest dose effective to produce a therapeutic effect. Such an effective dose will generally depend upon the factors described above.

In some embodiments, Compound 1 is formulated in an oral dosage form and administered to a patient orally. Oral dosage forms can be, for example, in the form of tablets, capsules, a liquid solution or suspension, a powder, or liquid or solid crystals, which contain the active agent in a mixture with physiologically acceptable excipients (e.g., pharmaceutically acceptable excipients). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other physiologically acceptable excipients (e.g., pharmaceutically acceptable excipients) can be colorants, flavoring agents, plasticizers, humectants, buffering agents, and the like.

In some embodiments, Compound 1 is formulated as a capsule. In some embodiments, the capsule comprises Compound 1 and at least one pharmaceutically acceptable excipient. In some embodiments, the at least one pharmaceutically acceptable excipient comprises hydroxypropyl methylcellulose (also known as Hypromellose), which is a semisynthetic, inert, viscoelastic polymer. In some embodiments, Compound 1 is formulated as an opaque, Hypromellose shell capsule. In some embodiments, the capsule is size 0 or size 2. In some embodiments, the capsule is size 2 and contains 0.5 mg of Compound 1. In some embodiments, the capsule is size 0 and contains 1 mg or 5 mg of Compound 1. In some embodiments, the capsule is orange (e.g., Swedish orange) in color. In some embodiments, the capsule is swallowed whole by the patient.

In some embodiments, Compound 1 is administered to the patient on an empty stomach, i.e., the patient does not consume any food 2 hours before or 1 hour after the dose of Compound 1. In some embodiments, Compound 1 is administered to the patient at approximately the same time on each treatment day.

In some embodiments, Compound 1 is administered to the patient on a continuous dosing schedule. The term “continuous dosing schedule,” as used herein, means that the dosing regimen is repeated continuously without any breaks over the course of the treatment period. In some embodiments, Compound 1 is administered to a patient once daily or twice daily on a continuous dosing schedule. In some embodiments, Compound 1 is administered to a patient continuously (e.g., on a once daily or twice daily regimen) for at least 3 consecutive days, 5 consecutive days, at least 7 consecutive days, at least 9 consecutive days, at least 14 consecutive days, at least 21 consecutive days, at least 28 consecutive days, or more. In some embodiments, Compound 1 is administered to a patient continuously (e.g., on a once daily or twice daily regimen) for one or more treatment cycles. In some embodiments, Compound 1 is administered to a patient continuously for at least 1, 2, 3, 4, 5, 6, or more treatment cycles. In some embodiments, one treatment cycle is 28 days.

A continuous dosing schedule differs from an intermittent dosing schedule, which has both treatment (i.e., “on”) and non-treatment (i.e., “off”) periods. Non-treatment periods may help to reduce the risk of drug-related toxicities that may be observed with continuous dosing (e.g., rash, neutropenia, thrombocytopenia). Non-treatment periods between cycles of treatment may also be referred to as “treatment breaks” or “treatment holidays.” In some embodiments, a treatment period may be at least 3 days, at least 5 days, at least 7 days, at least 9 days, at least 14 days, at least 21 days, at least 28 days, or more. In some embodiments, a non-treatment period may be at least 3 days, at least 5 days, at least 7 days, at least 9 days, at least 14 days, at least 21 days, at least 28 days, or more. In some embodiments, an intermittent dosing schedule alternates between treatment and non-treatment periods.

In some embodiments, Compound 1 is administered to a patient (e.g., on a once daily or twice daily regimen) on a 5 days on/9 days off dosing schedule. In some embodiments, this 14-day schedule is repeated once to complete one treatment cycle of 28 days. In some embodiments, Compound 1 is administered to a patient on a 5 days on/9 days off dosing schedule for one or more treatment cycles. In some embodiments, Compound 1 is administered to a patient on a 5 days on/9 days off dosing schedule for at least 1, 2, 3, 4, 5, 6, or more treatment cycles. In some embodiments, one treatment cycle is 28 days.

In some embodiments, Compound 1 is administered to a patient (e.g., on a once daily or twice daily regimen) on a 21 days on/7 days off dosing schedule. In some embodiments, Compound 1 is administered to a patient on a 21 days on/7 days off dosing schedule for one or more treatment cycles. In some embodiments, Compound 1 is administered to a patient on a 21 days on/7 days off dosing schedule for at least 1, 2, 3, 4, 5, 6, or more treatment cycles. In some embodiments, one treatment cycle is 28 days.

In some embodiments, Compound 1 is administered to the patient once daily. In some embodiments, Compound 1 is administered to the patient once daily on a 5 days on/9 days off dosing schedule. In some embodiments, Compound 1 is administered to the patient once daily on a 21 days on/7 days off dosing schedule. In some embodiments, Compound 1 is administered to the patient once daily on a continuous dosing schedule. In some embodiments, Compound 1 is administered to the patient once daily on a continuous dosing schedule until an adverse event or drug-related toxicity is observed. In some embodiments, a treatment holiday is incorporated into a once daily dosing schedule. In some embodiments, a treatment holiday is incorporated after at least about 5 days (e.g., after about 5 days, after about 7 days, after about 14 days, after about 21 days, or more) of once daily continuous dosing. In some embodiments, Compound 1 is administered to the patient once daily (e.g., continuously or intermittently) for one or more 28-day cycles.

In some embodiments, a therapeutically effective amount of Compound 1 is about 2 mg to about 20 mg given in a single dose on the day of administration. In some embodiments, the therapeutically effective amount of Compound 1 is about 2 mg, about 3.5 mg, about 5 mg, about 7 mg, about 10 mg, about 12 mg, about 14, or about 20 mg given in a single dose on the day of administration. In some embodiments, a dose lower than about 14 or 20 mg (e.g., 5 mg, 10 mg, or as low as about 2 mg once daily) reduces the risk of drug-related toxicities (e.g., cardiovascular events such as bradycardia and prolonged QTc), as compared to a higher dose.

In some embodiments, Compound 1 is administered to the patient twice daily. In some embodiments, Compound 1 is administered to the patient twice daily on a 5 days on/9 days off dosing schedule. In some embodiments, Compound 1 is administered to the patient twice daily on a 21 days on/7 days off dosing schedule. In some embodiments, Compound 1 is administered to the patient twice daily on a continuous dosing schedule. In some embodiments, Compound 1 is administered to the patient twice daily on a continuous dosing schedule until an adverse event or drug-related toxicity is observed. In some embodiments, a treatment holiday is incorporated into a twice daily dosing schedule. In some embodiments, a treatment holiday is incorporated after at least about 5 days (e.g., after about 5 days, after about 7 days, after about 14 days, after about 21 days, or more) of twice daily continuous dosing. In some embodiments, Compound 1 is administered to the patient twice daily (e.g., continuously or intermittently) for one or more 28-day cycles.

In some embodiments, a therapeutically effective amount of Compound 1 is a total of about 2 mg to about 20 mg given in two divided doses on the day of administration. In some embodiments, the therapeutically effective amount of Compound 1 is about 10 mg, about mg, or about 20 mg given in two divided doses on the day of administration. In some embodiments, the first dose is about 10 mg and the second dose is about 5 mg. In some embodiments, the first dose is about 5 mg and the second dose is about 10 mg. In some embodiments, the first dose and the second dose are each about 5 mg. In some embodiments, the first dose and the second dose are each about 7.5 mg. In some embodiments, the first dose and the second dose are each about 10 mg. In some embodiments, the interval between the first dose and the second dose is at least about 8 hours (e.g., about 8 hours, about 10 hours, about 12 hours).

In some embodiments, Compound 1 administered to the patient on a twice daily regimen (e.g., two doses of about 10 mg per dose) reduces the risk of drug-related toxicities, as compared to a higher dose on a once daily regimen (e.g., one dose of about 40 mg). In some embodiments, Compound 1 administered to the patient on a twice daily regimen reduces the risk of drug-related toxicities but achieves similar or higher biomarker modulation, as compared to a higher dose on a once daily regimen. For example, Compound 1 administered to the patient on a twice daily regimen may reduce a patient's TMEM14C AJ/CJ ratio for up to about hours while minimizing the risk of drug-related toxicities, as compared to a higher dose on a once daily regimen. In some embodiments of twice daily administration, the first dose and the second dose are each about 5 mg to about 10 mg, or more. In some embodiments, the first dose is about 10 mg and the second dose is about 5 mg. In some embodiments, the first dose is about 5 mg and the second dose is about 10 mg. In some embodiments, the first dose and the second dose are each about 5 mg. In some embodiments, the first dose and the second dose are each about 7.5 mg. In some embodiments, the first dose and the second dose are each about 10 mg. In some embodiments, the interval between the first dose and the second dose is at least about 8 hours (e.g., about 8 hours, about 10 hours, about 12 hours).

The dose of Compound 1 administered to the patient may be reduced over time. For example, at the start of treatment, Compound 1 may be administered at a dose of about 10 mg given twice daily, i.e., the first dose and the second dose are each about 10 mg. In some embodiments, the interval between the first dose and the second dose is about 10 hours to about 14 hours (e.g., about 10 hours, about 11 hours, about 12 hours, about 13 hours, about 14 hours). This daily dosing may then be reduced one or more times. In some embodiments, a first dose reduction comprises a first dose of about 5 mg and a second dose of about 10 mg, or vice versa. In some embodiments, a second or a subsequent dose reduction comprises a first dose and a second dose that are each about 5 mg.

In some embodiments, treatment with Compound 1 reduces or eliminates the patient's transfusion dependence. In some embodiments, treatment with Compound 1 reduces the number or frequency of transfusions given to the patient by at least about 10%, about 20%, about 30%, about 40%, about 50%, or about 60% as compared to the number or frequency prior to treatment. In some embodiments, treatment with Compound 1 reduces the number or frequency of transfusions given to the patient by at least about 30% as compared to the number or frequency prior to treatment. In some embodiments, treatment with Compound 1 reduces the number or frequency of transfusions given to the patient by at least about 60% as compared to the number or frequency prior to treatment. In some embodiments, the reduction in the number or frequency of transfusions observed with Compound 1 is greater than the reduction observed with an alternate treatment (e.g., a treatment described in List et al. (N Engl J Med. 2006;355(14):1456-1465), Fenaux et al. (Blood. 2011;118(14):3765-3776), or Fenaux et al. (N Engl J Med. 2020;382(2):140-151)). In some embodiments, the time period between transfusions observed with Compound 1 is longer than the time period between transfusions observed with an alternate treatment. In some embodiments, the reduction in the number or frequency of transfusions is measured over a period of at least 56 consecutive days (8 weeks), wherein the period begins any time after the start of treatment. In some embodiments, the patient does not receive any transfusions for a period of at least 56 consecutive days (8 weeks), wherein the period begins any time after the start of treatment. In some embodiments, the patient does not receive any transfusions for a period of at least 8 weeks, at least 9 weeks, at least 10 weeks, at least 12 weeks, at least 14 weeks, at least 16, or more, wherein the period begins any time after the start of treatment. In some embodiments, the reduction in the number or frequency of transfusions is measured over a period of at least 8 weeks or more during the first 24 weeks of treatment. In some embodiments, the reduction in the number or frequency of transfusions is measured over a period of at least 12 weeks or more during the first 24 weeks of treatment. In some embodiments, the reduction in the number or frequency of transfusions is measured over a period of at least 12 weeks or more during the first 48 weeks of treatment. In some embodiments, the reduction in the number or frequency of transfusions is measured over a period of at least 16 weeks or more during the first 24 weeks of treatment. In some embodiments, the reduction in the number or frequency of transfusions is measured over a period of at least 16 weeks or more during the first 48 weeks of treatment. In some embodiments, the transfusions comprise red blood cell (RBC) transfusions, platelet transfusions, or both. In some embodiments, the transfusions comprise RBC transfusions.

In some embodiments, treatment with Compound 1 increases the amount of bone marrow sideroblasts in the patient as compared to the amount prior to treatment. In some embodiments, treatment with Compound 1 increases the amount of bone marrow sideroblasts in the patient by at least about 10%, about 20%, about 30%, or about 40% as compared to the amount prior to treatment.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1. Assessment of Transfusion Independence in a Biomarker-Defined Subset of MDS Patients Treated with Compound 1

Compound 1, a small molecule that binds to SF3B1 and modulates pre-mRNA splicing, was tested in adult patients with myelodysplastic syndrome (MDS), chronic myelomonocytic leukemia (CMML), or acute myeloid leukemia (AML), including patients harboring splicing factor mutations and patients with wild type proteins (NCT02841540; see also Steensma et al. Blood (2019) 134 (Supplement 1): 673). Among 84 patients, 42 had MDS, 4 had CMML and 38 had AML. Dose escalation cohorts examined two once-daily dosing regimens: Schedule I (5 days on/9 days off) and Schedule II (21 days on/7 days off). Doses administered ranged from 1-40 mg on Schedule I (n=65) and 7-20 mg on Schedule II (n=19 patients); 25 patients (30%) received treatment for 1E30 days.

### Patients and Methods

### Study Design

Patients were enrolled using a conventional 3+3 dose escalation phase I design, with escalations based on a modified Fibonacci sequence scheme (Storer Biometrics. 1989;45(3):925-937). Dose escalations continued until a dose level at which ≥2 patients of the 3-6 enrolled experienced a dose-limiting toxicity (DLT). Maximally tolerated dose (MTD) was defined as the highest dose at which no more than 1 out of 6 patients experienced a DLT. DLTs were defined as any of the following: any Grade 3 or greater study drug-related non-hematologic toxicity except for Grade 3 nausea, vomiting, fatigue, or diarrhea that resolved to Grade 1 or less within 1 week; failure to administer at least 70% of the protocol-specific dose; or prolonged myelosuppression with the persistence of Grade 4 cytopenia in the absence of persistent leukemia or blast increase 21 or more days after suspending dosing. DLTs were assessed during the first 28 days using National Cancer Institute (NCI) Common Toxicity Criteria for Adverse Events (CTCAE) version 4.03.

The primary endpoints measured included occurrence of DLTs, the type and frequency of treatment-emergent adverse events (TEAEs), and serious adverse events (SAEs). Key secondary endpoints included drug pharmacokinetics (PK) and preliminary antitumor activity, such as the overall response rate (ORR), effect of drug therapy on transfusion requirements, and overall survival (OS). Biomarker analyses and drug pharmacodynamics (PD) were included as exploratory endpoints. The starting dose of 1 mg per day on a 5 day on, 9 day off schedule (Schedule I) was based on non-human primate experience; the DLT in that model system was gastrointestinal distress and colitis. In addition, a second schedule (Schedule II) of 21 days on therapy and a rest of 7 days without therapy was explored. The protocol was originally designed to evaluate Schedule I based on preclinical data suggesting activity of Compound 1 when administered intermittently. However, when limited clinical activity was observed on Schedule I, Schedule II was introduced to test whether more prolonged spliceosome modulation would lead to higher clinical activity. Each treatment cycle was 28 days in length. Patients could continue treatment until disease progression or development of unacceptable toxicity. Intra-patient dose escalation was permitted at Cycle 4 and beyond to dose levels demonstrated to be safe in other subsequently enrolled patients, but patients had to maintain their original dosing schedule.

### Inclusion Criteria

Eligibility criteria were disease-specific and are summarized in Table 1. In addition, patients had to be 18 years old, have an Eastern Cooperative Oncology Group (ECOG) performance status of 0-2, and adequate organ function, defined as: creatinine mg/dL or calculated creatinine clearance (Cockroft-Gault formula) ≥50 mL/min, direct bilirubin ≤1.5 times the upper limit of normal (ULN), alanine aminotransferase and aspartate aminotransferase ≤3.0×ULN, and albumin ≥2.5 mg/dL. Patients with MDS were further characterized using the International Prognostic Scoring System (IPSS) with “lower-risk” comprising IPSS Low and Intermediate-1 risk patients, collectively, versus “higher-risk” IPSS Intermediate-2 and High risk categories (Greenberg et al. Blood. 1997;89:2079-2088). Patients were not required to have a splicing mutation to be eligible.

TABLE 1
Disease-specific eligibility criteria

| Diagnosis | Diagnosis-Specific Eligibility Criteria |
|---|---|
| CMML | Treated with at least one prior therapy (e.g., hydroxyurea or an HMA) |
| Higher-risk MDS | Intolerant of HMAs in judgment of investigator/patient or |
| (IPSS Intermediate- | Not responded to 4 treatment cycles of decitabine or 6 treatment cycles |
| 2 or High) | of azacitidine or |
| | Progressed at any point after initiation of an HMA |
| Lower-risk MDS | Transfusion-dependent for RBCs or platelets |
| (IPSS Intermediate- | RBC transfusion-dependent patients must also have been failed by |
| 1 or Low) | ESAs or have serum erythropoietin level >500 U/L |
| | Platelet counts above 50 × 10⁹/L in the absence of transfusion for 8 |
| | weeks |
| AML | Declined or not considered a candidate for intensive induction |
| | chemotherapy by the enrolling physician |
| | Previously-treated patients should have evidence of persistent or |
| | recurrent AML in the peripheral blood and/or bone marrow that is |
| | refractory to, or has relapsed from, the most recent prior line of |
| | treatment |
| | WBC <15 × 10⁹/L |

AML, acute myeloid leukemia;
CMML, chronic myelomonocytic leukemia;
ESAs, erythropoiesis stimulating agents;
HMA, hypomethylating agent;
IPSS, International Prognostic Scoring System;
MDS, myelodysplastic syndrome;
RBC, red blood cell;
WBC, white blood cell.

### Ophthalmic Safety

Because visual loss was observed in subjects treated in a prior phase 1 trial of a pladienolide derivative (E7107) with chemical similarity to Compound 1 (Folco et al. Genes Dev. 2011;25(5):440-444; Hong et al. Invest New Drugs. 2014;32(3):436-444), and because germline mutation of the minor splicing factor PRPF8 and other related splicing factors is associated with retinitis pigmentosa (Grainger and Beggs, RNA. 2005;11(5):533-557), a detailed ophthalmologic safety plan was implemented. Eligibility criteria included normal vitamin A levels and visual acuity that corrected to 20/40 unless a cataract was present. Ophthalmologic evaluation, including fundoscopic imaging and visual evoked potentials, was performed during study screening and periodically throughout the duration of the trial.

### Response Assessment

Clinical responses were assessed using the 2006 International Working Group (IWG) response criteria for MDS (Cheson et al. Blood. 2006;108:419-425), the IWG 2003 criteria for AML, and the 2015 international consortium proposal of uniform response criteria for myelodysplastic/myeloproliferative neoplasms (MDS/MPN) and CMML (Savona et al. Blood. 2015;125(12):1857-1865). Peripheral blood sampling, bone marrow aspirates, bone marrow biopsies, marrow cytogenetics, and cellular composition by flow cytometry were performed at the time of screening, and after Cycles 1, 2, and 4. Beyond Cycle 4, bone marrow aspirates were performed as clinically indicated based on changes in peripheral blood count, or as needed to establish either complete response or disease progression. An independent central confirmation and interpretation was performed for disease assessments.

### Pharmacokinetics, Pharmacodynamics and Biomarker Analyses

Plasma samples for PK analyses were collected during Cycle 1 on Day 1 and Day 4 (pre-dose and 0.5, 1, 2, 4, 6, 8, 10, and 24 hours post-dose), and pre-dose and 4 hours post-dose on Cycle 1 on Day 15. PK analyses were conducted using Phoenix® VVinNonlin. For pharmacodynamic and biomarker analyses, peripheral blood samples were collected into PAXgene® Blood RNA Tubes (BD Biosciences, San Jose, California) at Cycle 1 Day 1 (pre-dose and 1, 2, 4, 10 and 24 hours post-dose) for all patients. RNA was extracted using Maxwell® simplyRNA Blood Kit (Promega, Madison, Wisconsin). Target engagement (i.e., splicing modulation) was measured by assessing relative expression of representative pre- and mature-mRNA or aberrant and canonical transcripts at post-dose time points comparing to pre-dose, using a customized Nanostring nCounter gene expression panel (NanoString Technologies, Seattle, Washington). For splicing mutation analysis, peripheral blood was collected into PAXgene® Blood DNA Tubes (BD Biosciences, San Jose, California). Baseline splicing mutations were identified using the Focus:Myeloid™ Next Generation Sequencing (NGS) panel determined centrally by Cancer Genetics Inc. (Rutherford, NJ). Biomarker analyses of pretreatment samples were conducted using one-way ANOVA and MedCalc® software. Receiver-Operating-Characteristics (ROC) curves were performed according to the methodology described by Hanley and McNeil (Radiology. 1982;143(1):29-36; see also Zweig and Campbell, Clin Chem. 1993;39(4):561-577).

### Results

### Enrolled Patients

A total of 84 patients were enrolled at 26 participating centers in the United States and Europe between October 2016 and December 2018. Of the enrolled patients, 65 were treated on Schedule I and 19 on Schedule II. The smaller number of patients on Schedule II reflects a later addition of this schedule as a protocol amendment after 7 dose levels on Schedule I had accrued without the observation of clinical responses. Baseline characteristics of enrolled patients are summarized in Table 2. Among enrolled patients, 38 patients had AML, 4 had CMML, 20 had IPSS higher-risk MDS, and 21 had lower-risk MDS. A normal karyotype was observed in 23 MDS patients and chromosome 8 trisomy, 7q deletion, 20q deletion and 5q deletion were observed, respectively, in 5, 4, 4, and 2 patients. Nine patients had other chromosomal abnormalities and 2 patients had a complex (3 or more abnormalities) karyotype. For 1 MDS patient, karyotyping failed so IPSS could not be calculated. AML with myelodysplasia-related changes was the most common AML diagnosis (N=20) whereas Refractory Anemia with Excess Blast was for MDS (N=21). CMML1/CMML2 diagnosis was not specified. Median number and range of prior regimens were 2 (1-9), 2 (2-5) and 2 (1-4) for AML, CMML and MDS patients, respectively. A total of 62 patients were reported to be RBC transfusion dependent in the 8 weeks prior to study entry.

Dose levels administered ranged from 1-40 mg on Schedule I and 7-20 mg on Schedule II. Enrollment of lower-risk MDS patients was suspended after a patient with SF3B1-mutant lower-risk MDS developed pancytopenia and marrow aplasia during the first week of study therapy at the 7 mg dose level (Schedule I, 5 days on/9 days off schedule). Thereafter, only AML, higher-risk MDS, and CMML patients were enrolled in the dose escalation portion of the trial. Among enrolled patients, 88% had a splicing mutation of interest (Tables 2 and 3).

TABLE 2
Baseline characteristics of enrolled patients

| Characteristic | n, (%) |
|---|---|
| Median age (range), y | 74 (46-87) |
| Male, n (%) | 61 (73) |
| Race, n (%) | |
| White | 72 (86) |
| Black or African American | 2 (2) |
| Asian | 2 (2) |
| Other or missing | 8 (10) |
| ECOG performance status, n (%) | |
| 0 | 18 (21) |
| 1 | 59 (70) |
| 2 | 7 (8) |
| Previous anticancer regimens, n (%) | |
| 0 | 3 (4) |
| 1 | 33 (39.3) |
| 2 | 24 (29) |
| ≥3 | 24 (29) |
| Prior anticancer regimens, median | 2 |
| Prior HMA treatment, n (%) | 73 (87) |
| Transfusion dependence,* n (%) | |
| RBC | 62 (71) |
| Platelet | 34 (41) |
| Disease | n (%) |
| MDS | 42 (50) † |
| Lower risk | 21 (25) |
| Higher risk | 20 (24) |
| IPSS missing | 1 (1) |
| CMML | 4 (5) |
| Lower risk | 1 (1) |
| Higher risk | 3 (4) |
| AML | 38 (45) |
| Splicing mutation | n (%) |
| SRSF2 | 34 (41) |
| SF3B1 | 25 (30) |
| U2AF1 | 13 (16) |
| ZRSR2 | 8 (10) |

*Assessed using International Working Group criteria.
† One patient had a diagnosis of RARS-T (MDS/MPN overlap syndrome).
AML, acute myeloid leukemia;
CMML, chronic myelomonocytic leukemia;
ECOG PS, Eastern Cooperative Oncology Group;
HMA, hypomethylating agent;
IPSS, International Prognostic Scoring System;
MDS, myelodysplastic syndrome;
RBC, red blood cell.

TABLE 3
Specific splicing mutations

| Splicing Mutation*, n (%) | N = 84 |
|---|---|
| SRSF2 | 34 (41) |
| p.P95H | 17 (20) |
| p.P95L | 9 (11) |
| p.P95_R102del | 4 (5) |
| Other mutations | 4 (5) |
| SF3B1 | 25 (30) |
| p.K700E | 11 (13) |
| p.R625C | 4 (5) |
| Other HEAT domain mutations | 10 (12) |
| U2AF1 | 13 (16) |
| p.Q157P | 6 (7) |
| p.S34F | 4 (5) |
| Other hotspot mutations | 3 (4) |
| ZRSR2, truncating or nonsense mutation | 8 (10) |

### Pharmacokinetics

Preliminary PK analysis indicated that Compound 1 is rapidly absorbed and exhibits dose-proportional increase in plasma exposure (FIG. 2). Key PK parameters via non-compartment analysis are listed in Table 4. A similar maximum cumulative dose per cycle was evaluated in both schedules (400 mg per 28 day cycle for Schedule I; 420 mg per 28 day cycle for Schedule II). Potential effect of covariates (including body weight and sex) on Compound 1 plasma PK were not evaluated due to the limited number of patients enrolled at each dose level on the 2 schedules; however, the overall variability in PK parameters was moderate, within a range typically observed in cancer patients, without obvious outliers (Table 4). A preliminary population PK modeling estimated the inter-individual variability on clearance/bioavailability was about 34%, suggesting that the potential covariate effect, if any, would likely not be meaningful, or that the currently available data are likely not sufficient to identify a meaningful covariate effect.

For Schedule I (doses tested ranged from 1 mg-40 mg), no obvious dose dependency in the rate of treatment related TEAEs (all grades) was observed. The incidence of these events ranged from 43% to 100% of subjects treated per dose level. At the lowest combined dose level (1, 2, 3.5 and 5 mg, N=25), 76% of subjects reported a treatment related TEAE. At the highest dose level tested (40 mg, N=6), 83% of subjects reported a treatment related TEAE. Grade 3 or higher treatment related TEAEs were reported in subjects treated at doses mg, with 3 of 6 subjects treated at 40 mg reporting grade 3 AEs. For Schedule II (7 mg-20 mg), treatment related AEs (all grades) were observed in 20% of subjects treated at the lowest dose level tested and 100% of subjects treated at the highest dose level tested, suggesting a dose dependency trend for AEs in the 21 day on/7 day off schedule. Grade 3 or higher treatment related TEAEs were reported in dose levels mg in Schedule II, with 3 of the 4 subjects treated at 20 mg reporting grade 3 AEs.

No obvious gender differences were observed in treatment related AEs, although 73% of subjects enrolled were male, so the assessment of potential gender differences on the tolerability of Compound I may be limited. Of the 63 subjects reporting treatment related AEs (all grades), 45 (71%) were male, and 9 (75%) of the 12 subjects who reported grade 3 treatment related AEs were male.

TABLE 4
Summary of Compound 1 plasma PK parameters on Day 4
following once-daily dose regimens
Statistical Summary of Plasma PK Parameters on Day 1

| Dose | | | | | AUC0-24 | |
|---|---|---|---|---|---|---|
| (mg) | n | Tmax (h) | | Cmax (μg/L) | (μg * h/L) | t1/2 (h) |
| 1.0 | 3 | 2 | (0.5-2) | 0.835 (90.8) | 4.89 (38.6) | 5.1 (65.3) |
| 2.0 | 7 | 2 | (0.5-6) | 1.09 (64.4) | 8.39 (42.0) | 5.4 (31.9) |
| 3.5 | 6 | 1.5 | (0.5-2) | 2.78 (82.4) | 15.6 (52.5) | 5.4 (15.2) |
| 5.0 | 9 | 1 | (0.5-4) | 3.94 (30.0) | 24.5 (24.2) | 5.8 (27.1) |
| 7.0 | 19 | 1 | (0.5-4) | 9.61 (69.7) | 39.4 (38.7) | 5.3 (23.8) |
| 10 | 7 | 1 | (0.5-2) | 12.7 (36.3) | 51.0 (20.5) | 5.5 (14.2) |
| 12 | 5 | 2 | (1-2) | 12.2 (37.4) | 64.7 (33.4) | 5.4 (17.3) |
| 14 | 10 | 1 | (0.5-2) | 25.6 (63.9) | 108 (55.1) | 5.1 (21.8) |
| 20 | 8 | 0.75 | (0.5-4) | 33.6 (49.2) | 142 (43.0) | 5.1 (10.3) |
| 30 | 4 | 1.25 | (0.5-2) | 34.4 (44.7) | 159 (23.9) | 5.3 (10.2) |
| 40 | 6 | 0.5 | (0.5-1) | 73.3 (85.8) | 265 (61.5) | 4.5 (17.2) |

Median (min-max) for Tmax and geometric mean (CV %) for other parameters

### Example 2. Further Analysis of Transfusion Independence in a Biomarker-Defined Subset of MDS Patients Treated with Compound 1

Further analysis of the data from the study described in Example 1 indicated the following, as set forth in this example.

### Further Analysis—Enrolled Patients

A total of 62 patients were reported to be RBC transfusion dependent in the 8 weeks prior to study entry.

### Further Analysis—Adverse Events and Dose-Limiting Toxicities

The most common treatment-related, treatment emergent adverse events (TEAEs, as judged by the investigator, ≥10% frequency) in the patients treated on Schedule I were diarrhea (42%), nausea (28%), fatigue (17%), and vomiting (14%) (Table 5). The most common treatment-related TEAEs in the patients treated on Schedule II were diarrhea (42%), vomiting (21%), QTcF (Fridericia method) prolongation (16%), nausea (16%), dysgeusia (11%), fatigue (11%), and hypophosphatemia (11%) (Table 5). Table 6 summarizes the most common Grade 3 and 4 events. One event of Grade 3 ocular papilledema was reported, without loss in visual acuity.

Altogether, 6 patients experienced an AE characterized as a DLT. On Schedule I, 1 patient with lower-risk MDS and SF3B1 mutation developed marrow aplasia at the 7 mg dose level, and 2 out of 6 patients at the 40 mg dose level were assessed by an investigator to have prolonged QTcF >500 msec. On Schedule II, 1 of 5 patients treated at the 14 mg dose level experienced grade 3 sinus bradycardia, and 2 of 4 patients treated at the 20 mg dose level experienced DLTs: Grade 3 QTc prolongation in one and Grade 3 nausea that did not promptly resolve in the other. Based on these results, 40 mg in schedule I and 20 mg in Schedule II appeared to exceed the DLT threshold, and the MTD was initially defined as 30 mg for Schedule I (cumulative dose 300 mg Compound I in 28-day cycles) and 14 mg for Schedule II (294 mg Compound I in 28-day cycles), and further dose escalation was stopped. An ad hoc independent cardiology review of patient ECGs was then undertaken in order to better understand the potential for cardiovascular effects with Compound I. Through this independent review, 2 of the QTc prolongation events reported as DLTs at 40 mg in Schedule I and 20 mg in Schedule II could not be confirmed, and the additional event at 40 mg was reported as potentially related to concomitant medications. Consequently, while the maximum tolerated dose (MTD) was not formally confirmed for either Schedule I or Schedule II, taking into account the totality of the data, a recommended Compound 1 QD dose was defined as 30 mg per day on Schedule I and 14 mg per day on Schedule II.

TABLE 5
Common treatment-related TEAEs (all grades) reported in ≥10% of patients by dosing schedule

| | Dose (QD), n (%) | | | | | | | | |
|---|---|---|---|---|---|---|---|---|---|
| | 1, 2, 3.5, & | | | | | | | | |
| Preferred Term | 5 mg | 7 mg | 10 mg | 12 mg | 14 mg | 20 mg | 30 mg | 40 mg | Total |
| Schedule I | N = 25 | N = 14 | N = 7 | N = 0 | N = 5 | N = 4 | N = 4 | N = 6 | N = 65 |
| Any TEAE | 19 (76) | 11 (79) | 3 (43) | — | 5 (100) | 3 (75) | 4 (100) | 5 (83) | 50 (77) |
| Diarrhea | 7 (28) | 4 (29) | 2 (29) | — | 5 (100) | 1 (25) | 3 (75) | 5 (83) | 27 (42) |
| Nausea | 3 (12) | 4 (29) | 2 (29) | — | 1 (20) | 2 (50) | 3 (75) | 3 (50) | 18 (28) |
| Fatigue | 3 (12) | 2 (14) | 1 (14) | — | 0 | 2 (50) | 1 (25) | 2 (33) | 11 (17) |
| Vomiting | 1 (4) | 2 (14) | 2 (29) | — | 1 (20) | 0 | 0 | 3 (50) | 9 (14) |
| Schedule il | N = 0 | N = 5 | N = 0 | N = 5 | N = 5 | N = 4 | N = 0 | N = 0 | N = 19 |
| Any TEAE | — | 1 (20) | — | 4 (80) | 4 (80) | 4 (100) | — | — | 13 (68) |
| Diarrhea | — | 1 (20) | — | 1 (20) | 2 (40) | 4 (100) | — | — | 8 (42) |
| Vomiting | — | 0 | — | 0 | 1 (20) | 3 (75) | — | — | 4 (21) |
| ECG QTcF prolonged | — | 0 | — | 0 | 1 (20) | 2 (50) | — | — | 3 (16) |
| Nausea | — | 0 | — | 1 (20) | 0 | 2 (50) | — | — | 3 (16) |
| Dysgeusia | — | 0 | — | 1 (20) | 0 | 1 (25) | — | — | 2 (11) |
| Fatigue | — | 0 | — | 1 (20) | 0 | 1 (25) | — | — | 2 (11) |
| Hypophosphatemia | — | 0 | — | 2 (40) | 0 | 0 | — | — | 2 (11) |

TABLE 6
Grade 3 or 4 treatment-related TEAEs reported in >2%
of patients by dosing schedule

| | Schedule I, n (%) | Schedule II, n (%) |
|---|---|---|
| Preferred Term | (N = 65) | (N = 19) |
| Anemia | 4 (6) | 0 |
| ECG QTcF prolonged | 2 (3) | 1 (5) |
| Fatigue | 2 (3) | 1 (5) |
| Platelet count decreased | 2 (3) | 1 (5) |
| Hypophosphatemia | 0 | 1 (5) |
| Nausea | 0 | 1 (5) |
| Sinus bradycardia | 0 | 1 (5) |

The severity of TEAEs was graded using NCI-CTCAE (version 4.03).
ECG, electrocardiogram; National Cancer Institute Common Terminology for Clinical Adverse Events; TEAE, treatment-emergent adverse event.

### Further Analysis—Duration of Treatment

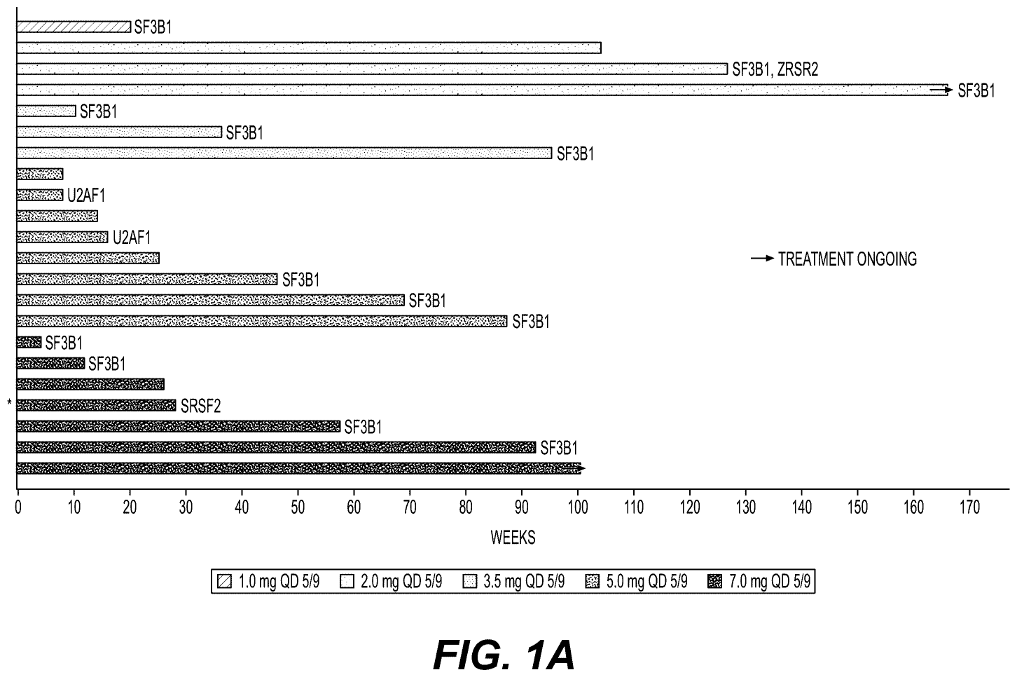

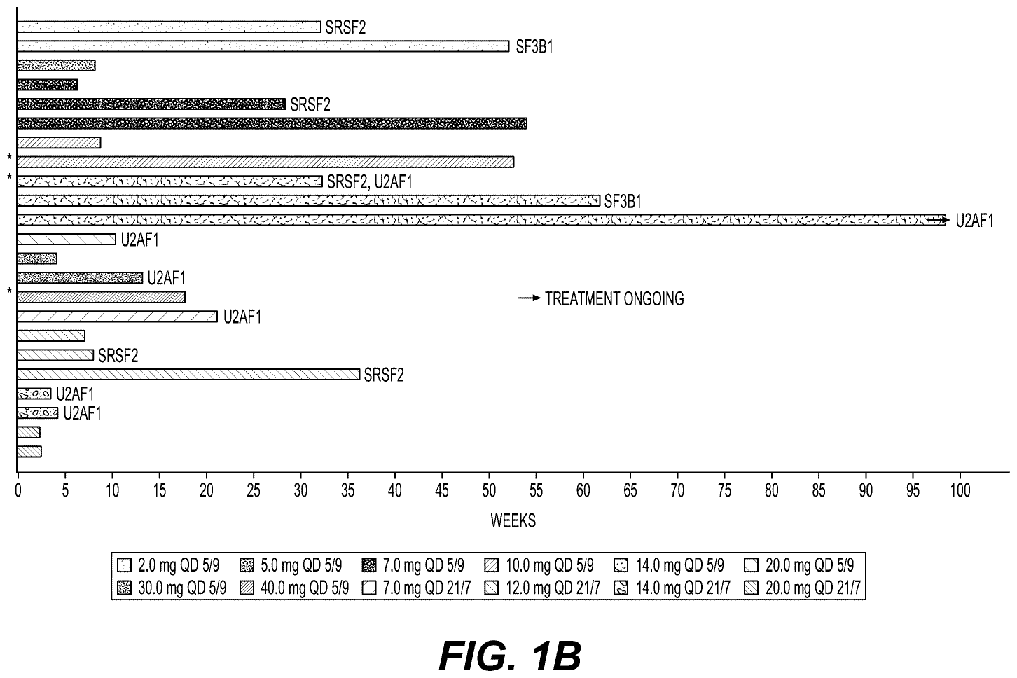

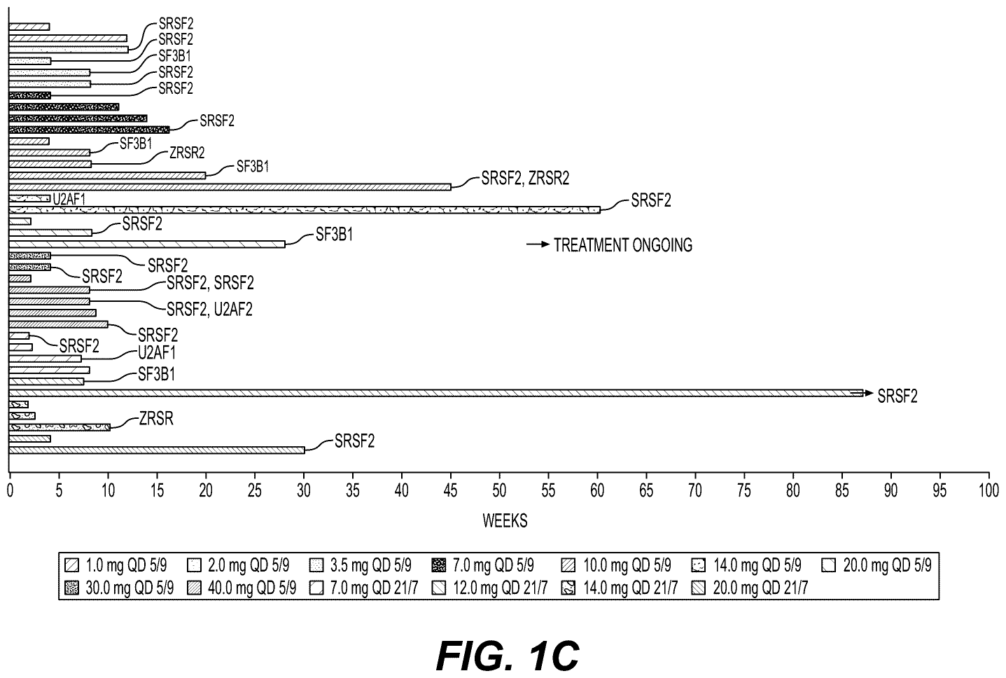

Patients remained on treatment from 12 to 1162 days; 27 patients (32%) had time on treatment greater than 180 days, 18% greater than 1 year, and 2% greater than 2 years (FIG. 1A-1C). The median duration of therapy for lower-risk MDS was 32.2 weeks, for higher-risk MDS/CMML was 13.0 weeks, and for AML was 8.0 weeks, reflecting the natural history of each disease.

### Further Analysis—Clinical Responses

No complete or partial responses meeting 2006 IWG criteria were observed. Based on MDS/MPN criteria, in the 4 subjects with a diagnosis of CMML, 1 complete cytogenetic remission and 1 clinical benefit (platelet response) were reported. No responses were observed in the remaining 2 CMML subjects. In addition, as of April 2020, 1 to 6 intervals of >56 days without RBC transfusions had been reported in 9 of 62 patients (15%) who were transfusion dependent at study entry according to IWG criteria 29 (Table 7). All of these patients met IWG criteria for a response of erythroid hematological improvement, except 1 who had a hemoglobin of 9.1 g/dL at baseline. All received Compound 1 within schedule I. 8 of the 9 patients had a first RBC TI that initiated in weeks 1-24 of the study. Of the 9 RBC TI cases, 1 was observed in a CMML subject (25%) and 8 in MDS (19%). The CMML patient also experienced a 21-week period of platelet transfusion independence. Four of the 9 patients experienced RBC TI at the 7 mg dose. Median duration of RBC TI was 13 weeks. Median time to onset of RBC TI was 15 weeks. Two additional patients (1 AML, 1 MDS) experienced RBC TI periods of >56 days but their transfusion dependence prior to study entry could not be verified. One case of a 22-week platelet transfusion independence without RBC TI was also observed in a higher risk MDS patient who received the 7 mg dose in Schedule II.

TABLE 7
Patients who experienced RBC TI ≥56 days

| | | | | Number | Longest | Time to |
|---|---|---|---|---|---|---|
| | | | Starting | of | RBC TI | First |
| Disease | | Age, | Dose | RBC TI | Period | RBC TI |
| Type | IPSS | Gender | (mg) | Periods | (days) | (days) |
| MDS | Higher | 78, M | 7 | 1 | 107 | 104 |
| MDS/ | Lower | 85, M | 2 | 1 | 56 | 111 |
| MPN^{†} | | | | | | |
| MDS | Lower | 68, M | 2** | 5 | 85 | 88 |
| MDS | Higher | 68, F | 2 | 1 | 121 | 126 |
| MDS | Lower | 80, M | 3.5 | 2 | 98 | 101 |
| MDS* | Lower | 69, M | 5 | 1 | 87 | 398 |
| CMML | Lower | 78, F | 7 | 1 | 66 | 4 |
| MDS | Lower | 72, F | 7 | 6 | 91 | 129 |
| MDS | Lower | 84, M | 7 | 4 | 98 | 73 |

^{†}RARS-T diagnosis.
*Baseline Hb was 9.1 g/dL.
**Patient received 7 mg of Compound 1 during his last two RBC TI periods.

TABLE 8
Listing of patients with missense SF3B1 mutations in
peripheral blood at Cycle 1 Day 1

| Age, Race, Gender | Diagnosis* | Histology* | SF3B1 |
|---|---|---|---|
| 58, W, M | MDS | RARS | p.K700E |
| 66, W, M | MDS | RARS | p.R625G |
| 61, W, M | MDS | RARS | p.K700E |
| 68, W, M | MDS | RARS | p.H662Q |
| 85, U, M | MDS | RARS | p.H662Q |
| 72, W, F | AML | AML with myelodysplasia | p.K700E |
| 81, W, M | MDS | RARS | p.K700E |
| 80, O, M | MDS | RARS | p.K700E |
| 69, W, M | MDS | RA | p.R625C |
| 82, W, M | AML | AML with maturation | p.K666N |
| 79, U, M | AML | AML with myelodysplasia | p.K700E |
| 71, W, M | N/E | N/E | p.K700E |
| 81, W, M | N/E | N/E | p.R625C |
| 84, W, M | MDS | RARS | p.K700E |
| 72, W, M | N/E | N/E | p.E592K |
| 76, W, M | MDS | RA | p.K700E |
| 72, W, F | AML | AML with myelodysplasia | p.R625H |
| 68, W, F | MDS | RAEB | p.R625C |
| 69, W, M | MDS | RARS | p.K700E |
| 72, W, M | MDS | RAEB | p.E622D |

*Based on central pathology assessment. Diagnosis was not confirmed in 2 MDS and 1 AML patients (N/E).
O, other;
W, white;
M, male;
U, unknown;
F, female;
MDS, myelodysplastic syndrome;
AML, acute myeloid leukemia;
N/E, non-evaluable;
RARS, refractory anemia with ring sideroblasts;
RA, refractory anemia.

### Further Analysis—Biomarkers

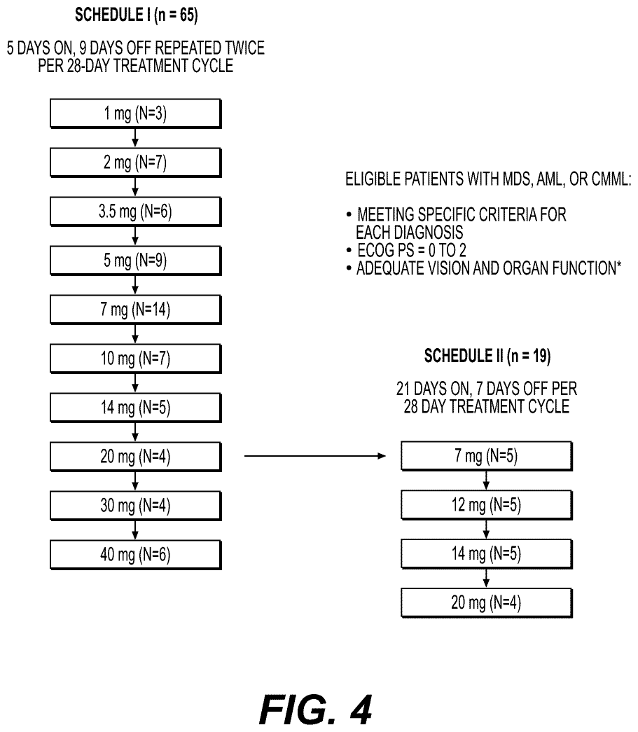

Mutation data on spliceosome proteins (SF3B1, SRSF2, U2AF1 and ZRSR2) were generated from PBMCs using 2 NGS panels in duplicate samples (FIG. 1A-C). SF3B1 mutations were the most common ones in MDS patients (15 missense mutations in 41 patients, 36.6%), particularly in lower risk MDS (57%) (FIG. 4). SF3B1 mutations were also observed in 5 of 38 (13%) AML patients and in none of the 4 CMML patients. A listing of patient characteristics for the subset of patients with missense SF3B1 mutations is shown in Table 8. There was insufficient sampling of PBMCs on study treatment to determine clonal changes in all of the patients who experienced RBC TI events. Of the 20 patients with missense SF3B1 mutations at study entry, 5 (25%) experienced RBC TI events. Three of the patients with RBC TI periods had a diagnosis of refractory anemia (RARS) with ring sideroblasts, 1 of refractory anemia with excess blasts and 1 of RARS with thrombocytosis. No RBC TI periods were observed in 4 patients with SF3B1 mutations and a diagnosis of AML.

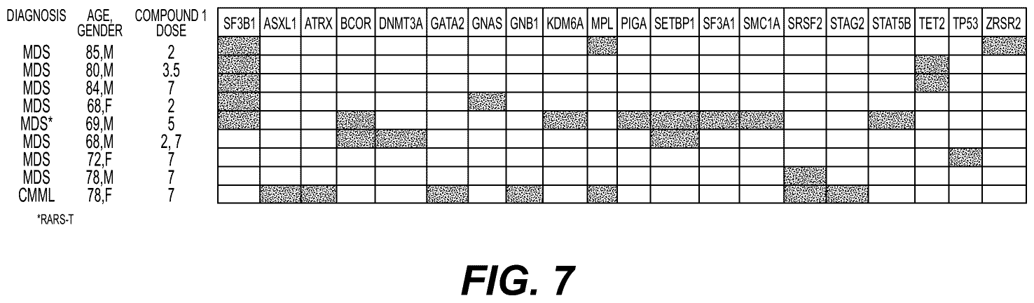

RBC TI periods on Compound I treatment were also observed in patients without SF3B1 mutations (FIG. 7), suggesting that modulation of splicing by wild type SF3B1 protein also plays a role in the mechanism of action of this agent in some patients and deserves further investigation. Of interest, patients with SF3B1 mutations appeared to have experienced RBC TI at lower doses than those with SF3B1 wild type status (FIG. 7).

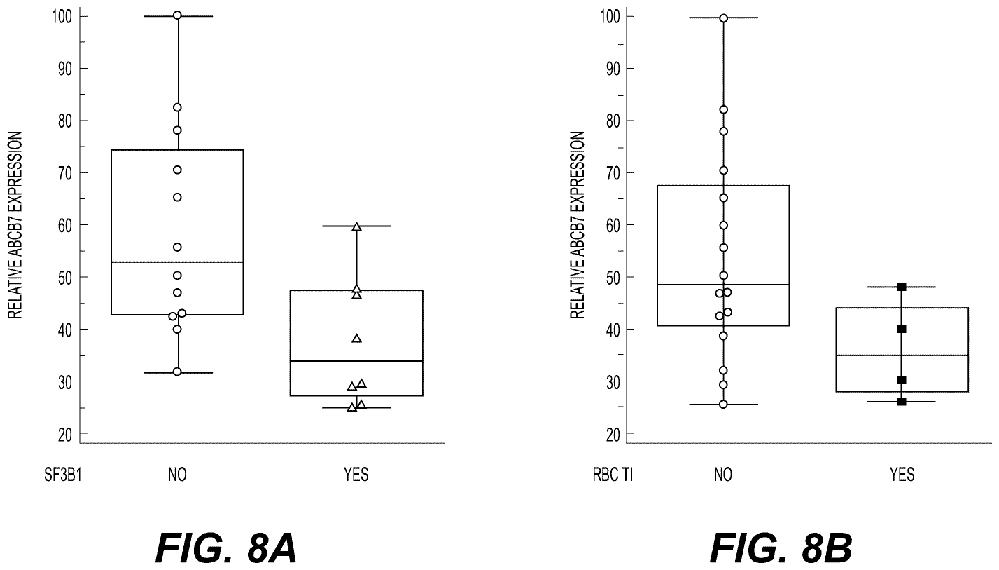

The relationship between ABCB7 expression (pre-treatment) and SF3B1 mutation status suggested that patients with SF3B1 mutations had lower ABCB7 expression, as measured by RT-PCR (FIG. 8A). Furthermore, the relationship between ABCB7 expression (pre-treatment) and the incidence of RBC TI while on Compound 1 treatment or during treatment follow up suggested patients with lower ABCB7 expression had a higher instance of RBC TI (FIG. 8B).

Mutated SF3B1 has been associated with the ring sideroblast phenotype, which is characterized by defects in heme biosynthesis and iron accumulation in mitochondria. Three genes involved in heme biosynthesis and iron metabolism are recurrently aberrantly spliced in SF3B1-mutated patients with MDS: ABCB7, PPOX, and TMEM14C (Shiozawa et al. Nat Commun. 2018;9:3649). PPOX works with TMEM14C to facilitate the mitochondrial transport of porphyrins. It is possible that modulation of ABCB7 or PPOX could contribute to Compound I induced RBC TI and should be investigated in future studies.

TABLE 9
Splicing markers

| | | | | Junctions | Junctions | Junctions |
|---|---|---|---|---|---|---|
| | | | | affected by | affected by | affected by |
| | | | | SF3B1 | U2AF1 | SRSF2 |
| Junctions that may not be associated with a particular mutation | | | | mutations | mutations | mutations |
| COASY-mat | PLEKHJ1-mat | PWP1-mat | CDKN1A-mat | TMEM14C_CJ | EFCAB14_e6- | RHOT2-CJ |
| | | | | | e8 (AJ_1) | |
| COASY-pre | PLEKHJ1-pre_1 | PWP1-pre | CDKN1A-pre | TMEM14C_AJ | EFCAB14_e7- | RHOT2-AJ |
| | | | | | e8 (CJ_1) | |
| EIF4A1-mat | PLEKHJ1-pre_2 | DYNLT1-mat | OGFOD2-mat | ZDHHC16_CJ | IRAK4_e4_e5 | CEP57-CJ |
| | | | | | (AJ_3) | |
| EIF4A1-pre | RBM5-mat | DYNLT1-pre | OGFOD2-pre | ZDHHC16_AJ | IRAK4_e3_e5 | CEP57-AJ |
| | | | | | (CJ_3) | |
| FBXW5-mat | RBM5-pre_1 | | TRIM65-mat | SLTM_CJ | TEX30_e5-e6 | |
| | | | | | (AJ_2) | |
| FBXW5-pre | RBM5-pre_2 | | TRIM65-pre | SLTM_AJ | TEX30_e4-e6 | |
| | | | | | (CJ_3) | |
| MBD4-mat | ADPRHL2-mat | | UNC50-mat | SNURF_CJ | UBA2_e1-e3 | |
| | | | | | (AJ_4) | |
| MBD4-pre | ADPRHL2-pre | | UNC50-pre | SNURF_AJ | UBA2_e1-e2 | |
| | | | | | (CJ_1) | |
| SLC25A19-mat | ADPRHL2-pre_2 | | KRI1-mat | ZNF561_AJ | WSB1_Intron- | |
| (e3-4) | | | | | e6 | |
| | | | | | (AJ_2) | |
| SLC25A19-mat | CDK9-mat | | KRI1-pre | TAK1_CJ | WSB1_e6 | |
| (e5-6) | | | | | (CJ_1) | |
| DPH2-mat | CDK9-pre | | | ZNF410_CJ | | |
| (e2-3) | | | | | | |

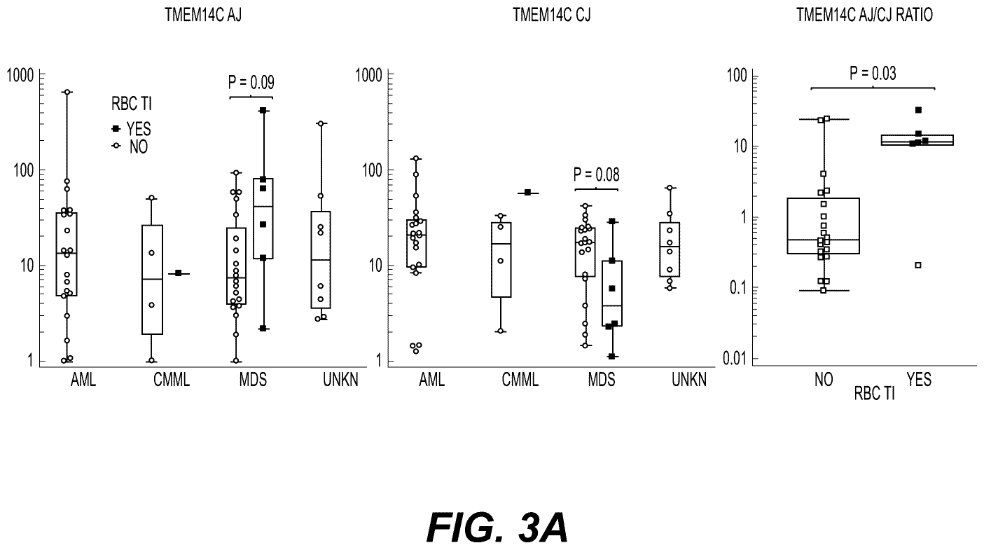

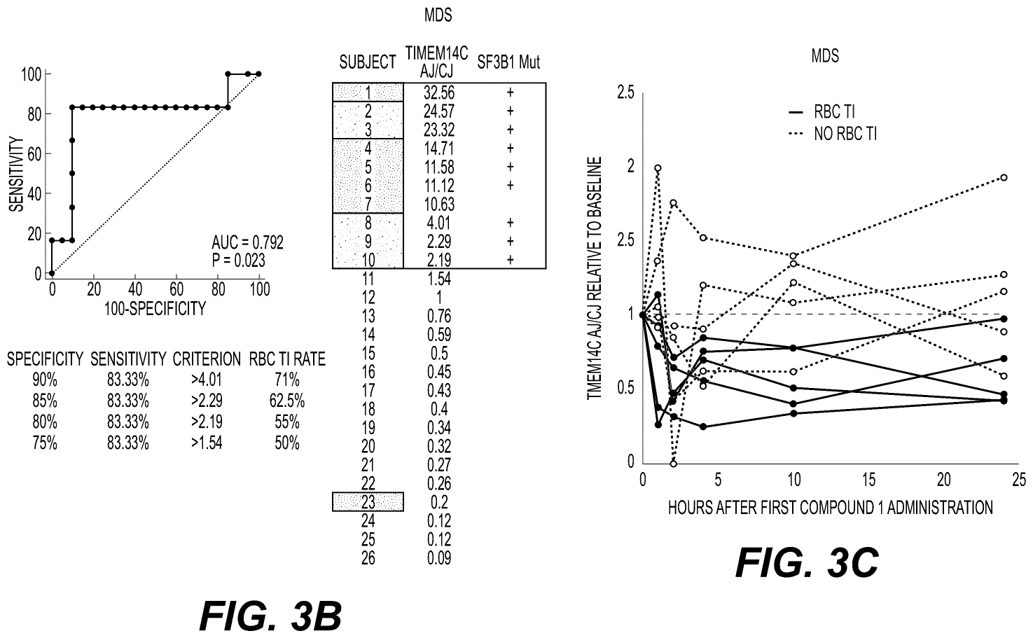

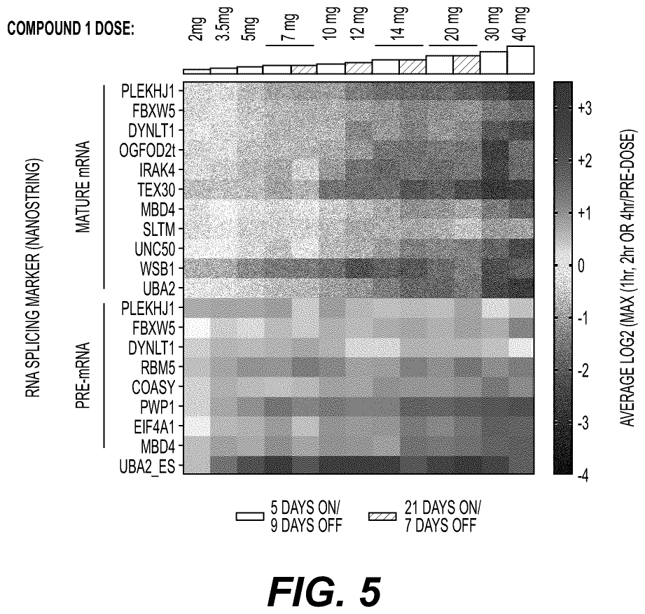

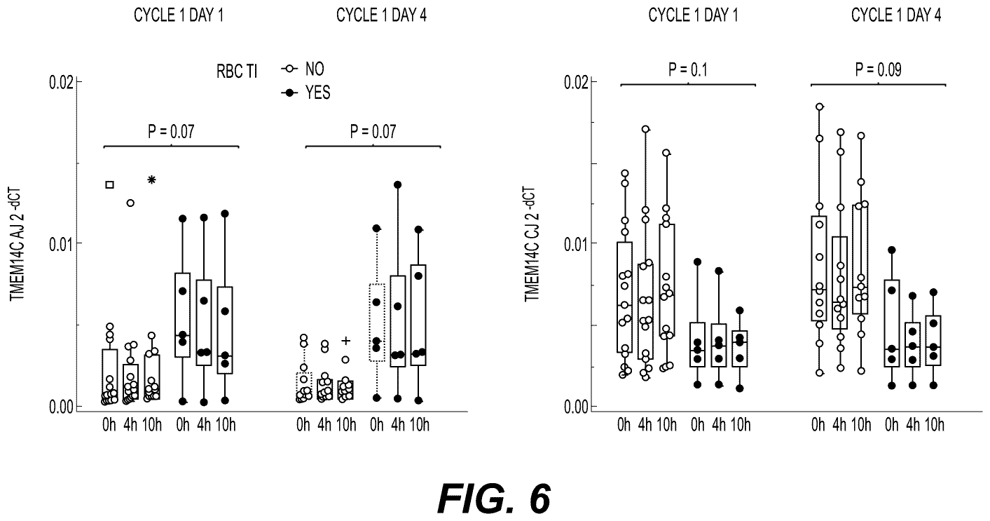

PBMC samples for pharmacodynamics assessments were collected from 59 of the 84 patients, including 26 pre-treatment samples from MDS patients, and gene expression data generated using NanoString probes. A total of 61 splicing markers were investigated (Table 9). A general modulation of splicing markers post-dosing was observed (FIG. 5). Seven patients who experienced RBC TI >56 days had available gene expression data. Trends for increased pre-treatment aberrant splicing junction (AJ) transcripts and decreased pre-treatment canonical splicing junction (CJ) transcripts of the gene encoding for TMEM14C were observed in the MDS patients who experienced RBC TI (FIG. 3A). ROC curve analyses indicated that the pre-treatment ratio of TMEM14C AJ/CJ was predictive of the onset of RBC TI on Compound I treatment in MDS patients with an optimal Youden index J=0.733 for an associated criterion of >4.01 with a 83.3% sensitivity and 90% specificity. Of the 7 MDS patients with (TMEM14C AJ/CJ >4.01, FIG. 3B), 5 experienced events of RBC TI with Compound I (71%). SF3B1 mutations were detected in all but 1 of them (FIG. 3B). Downregulation of the TMEM14C AJ/CJ ratio with Compound I dosing was also observed in the patients who experienced RBC TI, with a nadir at 2-10 hours (FIG. 3C). Because of the potential relevance of these findings, quantification of TMEM14C aberrant and canonical transcripts were repeated using RT-qPCR in residual study samples (N=20, including 4 patients who experienced RBC TI). Additional time points were also included in these experiments (Cycle 1 Day 4). Pre-treatment expression of TMEM14C AJ was higher in Cycle 1 Day 1 PBMC samples from patients who experienced RBC TI with Compound I treatment (FIG. 6). Likewise, pre-treatment expression of TMEM14C AJ was also higher in Cycle 1 Day 4 samples from those patients (FIG. 6).

Selected Sequences:

```
Canonical sequence for TMEM14C (SEQ ID NO: 1)
CCGGGGCCTTCGTGAGACCGGTGCAGG CCTGGGGTAGTCT
Aberrant sequence for TMEM14C (SEQ ID NO: 2)
CCGGGGCCTTCGTGAGACCGCTTG TTTTCTGCAGGTGCAG
Human SF3B1-Amino acid sequence (SEQ ID NO: 3)
MAKIAKTHEDIEAQIREIQGKKAALDEAQGVGLDSTGYYDQEIYGGSDSRFAGYVTSIAATELEDDDDDYSSSTS
LLGQKKPGYHAPVALLNDIPQSTEQYDPFAEHRPPKIADREDEYKKHRRTMIISPERLDPFADGGKTPDPKMNAR
TYMDVMREQHLTKEEREIRQQLAEKAKAGELKVVNGAAASQPPSKRKRRWDQTADQTPGATPKKLSSWDQAETPG
HTPSLRWDETPGRAKGSETPGATPGSKIWDPTPSHTPAGAATPGRGDTPGHATPGHGGATSSARKNRWDETPKTE
RDTPGHGSGWAETPRTDRGGDSIGETPTPGASKRKSRWDETPASQMGGSTPVLTPGKTPIGTPAMNMATPTPGHI
MSMTPEQLQAWRWEREIDERNRPLSDEELDAMFPEGYKVLPPPAGYVPIRTPARKLTATPTPLGGMTGFHMQTED
RTMKSVNDQPSGNLPFLKPDDIQYFDKLLVDVDESTLSPEEQKERKIMKLLLKIKNGTPPMRKAALRQITDKARE
FGAGPLFNQILPLLMSPTLEDQERHLLVKVIDRILYKLDDLVRPYVHKILVVIEPLLIDEDYYARVEGREIISNL
AKAAGLATMISTMRPDIDNMDEYVRNTTARAFAVVASALGIPSLLPFLKAVCKSKKSWQARHTGIKIVQQIAILM
GCAILPHLRSLVEIIEHGLVDEQQKVRTISALAIAALAEAATPYGIESFDSVLKPLWKGIRQHRGKGLAAFLKAI
GYLIPLMDAEYANYYTREVMLILIREFQSPDEEMKKIVLKVVKQCCGTDGVEANYIKTEILPPFFKHFWQHRMAL
DRRNYRQLVDTTVELANKVGAAEIISRIVDDLKDEAEQYRKMVMETIEKIMGNLGAADIDHKLEEQLIDGILYAF
QEQTTEDSVMLNGFGTVVNALGKRVKPYLPQICGTVLWRLNNKSAKVRQQAADLISRTAVVMKTCQEEKLMGHLG
VVLYEYLGEEYPEVLGSILGALKAIVNVIGMHKMTPPIKDLLPRLTPILKNRHEKVQENCIDLVGRIADRGAEYV
SAREWMRICFELLELLKAHKKAIRRATVNTFGYIAKAIGPHDVLATLLNNLKVQERQNRVCTTVAIAIVAETCSP
FTVLPALMNEYRVPELNVQNGVLKSLSFLFEYIGEMGKDYIYAVTPLLEDALMDRDLVHRQTASAVVQHMSLGVY
GFGCEDSLNHLLNYVWPNVFETSPHVIQAVMGALEGLRVAIGPCRMLQYCLQGLFHPARKVRDVYWKIYNSIYIG
SQDALIAHYPRIYNDDKNTYIRYELDYIL
Human SF3B1-Nucleic acid sequence (SEQ ID NO: 4)
AGTTCCGTCTGTGTGTTCGAGTGGACAAAATGGCGAAGATCGCCAAGACTCACGAAGATATTGAAGCACAGATTC
GAGAAATTCAAGGCAAGAAGGCAGCTCTTGATGAAGCTCAAGGAGTGGGCCTCGATTCTACAGGTTATTATGACC
AGGAAATTTATGGTGGAAGTGACAGCAGATTTGCTGGATACGTGACATCAATTGCTGCAACTGAACTTGAAGATG
ATGACGATGACTATTCATCATCTACGAGTTTGCTTGGTCAGAAGAAGCCAGGATATCATGCCCCTGTGGCATTGC
TTAATGATATACCACAGTCAACAGAACAGTATGATCCATTTGCTGAGCACAGACCTCCAAAGATTGCAGACCGGG
AAGATGAATACAAAAAGCATAGGCGGACCATGATAATTTCCCCAGAGCGTCTTGATCCTTTTGCAGATGGAGGGA
AAACCCCTGATCCTAAAATGAATGCTAGGACTTACATGGATGTAATGCGAGAACAACACTTGACTAAAGAAGAAC
GAGAAATTAGGCAACAGCTAGCAGAAAAAGCTAAAGCTGGAGAACTAAAAGTCGTCAATGGAGCAGCAGCGTCCC
AGCCTCCATCAAAACGAAAACGGCGTTGGGATCAAACAGCTGATCAGACTCCTGGTGCCACTCCCAAAAAACTAT
CAAGTTGGGATCAGGCAGAGACCCCTGGGCATACTCCTTCCTTAAGATGGGATGAGACACCAGGTCGTGCAAAGG
GAAGCGAGACTCCTGGAGCAACCCCAGGCTCAAAAATATGGGATCCTACACCTAGCCACACACCAGCGGGAGCTG
CTACTCCTGGACGAGGTGATACACCAGGCCATGCGACACCAGGCCATGGAGGCGCAACTTCCAGTGCTCGTAAAA
ACAGATGGGATGAAACCCCCAAAACAGAGAGAGATACTCCTGGGCATGGAAGTGGATGGGCTGAGACTCCTCGAA
CAGATCGAGGTGGAGATTCTATTGGTGAAACACCGACTCCTGGAGCCAGTAAAAGAAAATCACGGTGGGATGAAA
CACCAGCTAGTCAGATGGGTGGAAGCACTCCAGTTCTGACCCCTGGAAAGACACCAATTGGCACACCAGCCATGA
ACATGGCTACCCCTACTCCAGGTCACATAATGAGTATGACTCCTGAACAGCTTCAGGCTTGGCGGTGGGAAAGAG
AAATTGATGAGAGAAATCGCCCACTTTCTGATGAGGAATTAGATGCTATGTTCCCAGAAGGATATAAGGTACTTC
CTCCTCCAGCTGGTTATGTTCCTATTCGAACTCCAGCTCGAAAGCTGACAGCTACTCCAACACCTTTGGGTGGTA
TGACTGGTTTCCACATGCAAACTGAAGATCGAACTATGAAAAGTGTTAATGACCAGCCATCTGGAAATCTTCCAT
TTTTAAAACCTGATGATATTCAATACTTTGATAAACTATTGGTTGATGTTGATGAATCAACACTTAGTCCAGAAG
AGCAAAAAGAGAGAAAAATAATGAAGTTGCTTTTAAAAATTAAGAATGGAACACCACCAATGAGAAAGGCTGCAT
TGCGTCAGATTACTGATAAAGCTCGTGAATTTGGAGCTGGTCCTTTGTTTAATCAGATTCTTCCTCTGCTGATGT
CTCCTACACTTGAGGATCAAGAGCGTCATTTACTTGTGAAAGTTATTGATAGGATACTGTACAAACTTGATGACT
TAGTTCGTCCATATGTGCATAAGATCCTCGTGGTCATTGAACCGCTATTGATTGATGAAGATTACTATGCTAGAG
TGGAAGGCCGAGAGATCATTTCTAATTTGGCAAAGGCTGCTGGTCTGGCTACTATGATCTCTACCATGAGACCTG
ATATAGATAACATGGATGAGTATGTCCGTAACACAACAGCTAGAGCTTTTGCTGTTGTAGCCTCTGCCCTGGGCA
TTCCTTCTTTATTGCCCTTCTTAAAAGCTGTGTGCAAAAGCAAGAAGTCCTGGCAAGCGAGACACACTGGTATTA
AGATTGTACAACAGATAGCTATTCTTATGGGCTGTGCCATCTTGCCACATCTTAGAAGTTTAGTTGAAATCATTG
AACATGGTCTTGTGGATGAGCAGCAGAAAGTTCGGACCATCAGTGCTTTGGCCATTGCTGCCTTGGCTGAAGCAG
CAACTCCTTATGGTATCGAATCTTTTGATTCTGTGTTAAAGCCTTTATGGAAGGGTATCCGCCAACACAGAGGAA
AGGGTTTGGCTGCTTTCTTGAAGGCTATTGGGTATCTTATTCCTCTTATGGATGCAGAATATGCCAACTACTATA
CTAGAGAAGTGATGTTAATCCTTATTCGAGAATTCCAGTCTCCTGATGAGGAAATGAAAAAAATTGTGCTGAAGG
TGGTAAAACAGTGTTGTGGGACAGATGGTGTAGAAGCAAACTACATTAAAACAGAGATTCTTCCTCCCTTTTTTA
AACACTTCTGGCAGCACAGGATGGCTTTGGATAGAAGAAATTACCGACAGTTAGTTGATACTACTGTGGAGTTGG
CAAACAAAGTAGGTGCAGCAGAAATTATATCCAGGATTGTGGATGATCTGAAAGATGAAGCCGAACAGTACAGAA
AAATGGTGATGGAGACAATTGAGAAAATTATGGGTAATTTGGGAGCAGCAGATATTGATCATAAACTTGAAGAAC
AACTGATTGATGGTATTCTTTATGCTTTCCAAGAACAGACTACAGAGGACTCAGTAATGTTGAACGGCTTTGGCA
CAGTGGTTAATGCTCTTGGCAAACGAGTCAAACCATACTTGCCTCAGATCTGTGGTACAGTTTTGTGGCGTTTAA
ATAACAAATCTGCTAAAGTTAGGCAACAGGCAGCTGACTTGATTTCTCGAACTGCTGTTGTCATGAAGACTTGTC
AAGAGGAAAAATTGATGGGACACTTGGGTGTTGTATTGTATGAGTATTTGGGTGAAGAGTACCCTGAAGTATTGG
GCAGCATTCTTGGAGCACTGAAGGCCATTGTAAATGTCATAGGTATGCATAAGATGACTCCACCAATTAAAGATC
TGCTGCCTAGACTCACCCCCATCTTAAAGAACAGACATGAAAAAGTACAAGAGAATTGTATTGATCTTGTTGGTC
GTATTGCTGACAGGGGAGCTGAATATGTATCTGCAAGAGAGTGGATGAGGATTTGCTTTGAGCTTTTAGAGCTCT
TAAAAGCCCACAAAAAGGCTATTCGTAGAGCCACAGTCAACACATTTGGTTATATTGCAAAGGCCATTGGCCCTC
ATGATGTATTGGCTACACTTCTGAACAACCTCAAAGTTCAAGAAAGGCAGAACAGAGTTTGTACCACTGTAGCAA
TAGCTATTGTTGCAGAAACATGTTCACCCTTTACAGTACTCCCTGCCTTAATGAATGAATACAGAGTTCCTGAAC
TGAATGTTCAAAATGGAGTGTTAAAATCGCTTTCCTTCTTGTTTGAATATATTGGTGAAATGGGAAAAGACTACA
TTTATGCCGTAACACCGTTACTTGAAGATGCTTTAATGGATAGAGACCTTGTACACAGACAGACGGCTAGTGCAG
TGGTACAGCACATGTCACTTGGGGTTTATGGATTTGGTTGTGAAGATTCGCTGAATCACTTGTTGAACTATGTAT
GGCCCAATGTATTTGAGACATCTCCTCATGTAATTCAGGCAGTTATGGGAGCCCTAGAGGGCCTGAGAGTTGCTA
TTGGACCATGTAGAATGTTGCAATATTGTTTACAGGGTCTGTTTCACCCAGCCCGGAAAGTCAGAGATGTATATT
GGAAAATTTACAACTCCATCTACATTGGTTCCCAGGACGCTCTCATAGCACATTACCCAAGAATCTACAACGATG
ATAAGAACACCTATATTCGTTATGAACTTGACTATATCTTATAATTTTATTGTTTATTTTGTGTTTAATGCACAG
CTACTTCACACCTTAAACTTGCTTTGATTTGGTGATGTAAACTTTTAAACATTGCAGATCAGTGTAGAACTGGTC
ATAGAGGAAGAGCTAGAAATCCAGTAGCATGATTTTTAAATAACCTGTCTTTGTTTTTGATGTTAAACAGTAAAT
GCCAGTAGTGACCAAGAACACAGTGATTATATACACTATACTGGAGGGATTTCATTTTTAATTCATCTTTATGAA
GATTTAGAACTCATTCCTTGTGTTTAAAGGGAATGTTTAATTGAGAAATAAACATTTGTGTACAAAATGCTAATT
TGTGTGTGTTTTTTGAACATGACTTGTAAAATGCGGAACTTTGATAAAGTATTGGTTTATGTGGAATAAGTGGCT
TAATTTCATTTTCTGTCACATGGTTTATAGAAAGTAGTTAGCTGAATAAAAACTATATAAAGTGATGGCATCTTT
GTCAAAATTCCATTGTTGTGTTAATAATGACAGCAAGAAGAGTAGCCTCAGGGGATGTTCCCCTCAAACTAGCAC
AACCATTCCATCATCGTAGAAAAGTAGCACTTTTGCTAAACTGTCTTGAATATTTTGTACTTACATAGCGCCTTT
CATCTCTTGATTTCTCAAAATGCTTTATGAACACATTTAAAGAAAGTGGTTTAAGTCTTGTCCAACACTTGACAG
GTCTGCTGTGTTTAGCAAGTGAGGAATTTAACTTTACTTCAAAACTGCTTTCTGCCTATTAGGAGTGAGGATACC
TAAGTAATGCTGATAGAACAGGACAATGTTGGGCTTTTCTCCATGTTATAAGCCACTACTCAGCAATGCATCAGT
AAATACCTATTCACCCACTGTATGCCAGCCACTGTGCTTATATGCAGGGGATGCAAAGGTGCATGAGACCTGCCC
TCTACCTTTAAGAACAGTATAATGGAGAAGGGGAGACAAACCTTGTGACAGATTCCTTATGTACTGTGATAATAC
ACAGTGGCAGCAGCTAAAATGTCTAGGTTTGCTTAGCTTTGTATTCAGTAATAATAAGTTGATCTAAGAGTTCAG
CATAAACTGAATGAAATGCCATTTAATGGTAGAGGAACCAAGCATTAAGGCAGTACTACACTTAATTTTTTAAGC
AAATATGTAAAGTATATTTTCAAACTTTTCTAATGTTATGGCCCCAAATTTCTTAGTTTTGGCCTTTTTACTACC
TATACTATTTTTACTGTTTTGTTTTGTCTCCATGTGGTAGTACTTCTGTGAACTCTAAAGGGAAAAAAAATTCTG
CAAGCAGCATTAGTATATAACTACTACTGTAAGTAAAACTGCCTATTGACACTTTAGGAGTTCCTGCTTCAGAAG
CTTAGTTAAGAAACAGCTTGTGGCCGGGTGTGGTGGCTCATGCCTGTAATCCCAGCACTTTGGGAGGCTGAGGCG
GGCGGATCACGAGGTCAGGAGATCGAGACCATCCTGGCTAACGCGGTGAAGTCCCGTCTACTAAAAATACAAAAA
TTAGCCAGGCGTGGTGGCGGGCGCCTGTAGTCCCAGCTACTAGGGAGGCTGAGGCAGGAGAATGGCTTGAACCCG
GGAGGCGGAGCTTGCAGTGAGCCGAGATAGCGCTGCTACACTCCAGCCTGGGCGACAGAGACTCCGTCTCAAAAA
AAAAAAAAAAATTGACTTTCAACAAATTGATAGTGAGCATTAAGGGTTTCCAAGTTGGATTTGTAACTCCTCATC
ATTCCTTGTATGACAACTTTCTGAATATATGTCACTATGTAGTAAAATTAAACACTCCAAACTCATCTTTCTGTT
GTTAGAAGTTTTCAGCGGTACTTCCATGCAACTTTAAATCTCACTGCTCTCTATGGTTGATGTCAAATGACCTTC
AGTAATGACTGAGAATTGAATACAAATAGATTACAAAGCCAAAATTTGATGTTAAATGACTCAGGAAATTTTAGT
TGTATTTTCAATTCAAGTACTTAGTAGCCTACGTTTGCTTGGCCTCTGGTTCTTTATGGAAAATAGGCTTTGTAG
TGGCATTGTGGAGCAAAGGAGACTGTTACACCTTAATTAACTTTTTTTACTGATGCAAATAATTTGAGGATAGAG
AGGAGGGAAGTAGTGAAAGCTATGACCTAAAACATTGGGACCAAATAGAGGCTCACAGATATTTGGATTATTTTA
TGTGCTTATTATTAAATAAGGAAAGCATTTTGTGATATGTGGAAGACGCTATGTGAAGTTTTACCTATCTTCTCA
AAGACCTTTTCTTTTGTATTTTCTTTTGGTGTTTCTTAAAGCCAAACAAAGAAATGTTCTTAAGGAGACAGGGTG
GGTTTTTCTGTGGGCCTTTGTTGGTTTTTCTGTGGGCCATCGCCCTCTAATGGAATTGATCTCTGGCTGTTTGAT
TTTTTTCATATTGTATTTTTAAAATTTGTTGTACAGTGCCCTGTGAGCACCAAGTACCACTAGATGAATAAAACG
TATTATATCTAAA
TMEM14C-Canonical and aberrant splicing sites (SEQ ID NO: 5)
CTG [symbol] CTGCTGGAGAGCTGTGCTTTTA [symbol] CTACCTCTGATCC [symbol] CTTGTTTTCTGCAG
                                                  −16 -−20
AG = canonical 3′ splice site [symbol]
= cryptic 3′ splice site
A = SVM-BP predicted canonical branch point [symbol]
= SVM-BP predicted cryptic branch point
ABCB7-Canonical and aberrant splicing sites (SEQ ID NO: 6)
CACTTAAGAGAATAAATTCTA [symbol] TTATACTTGCCTAT [symbol] TTATTTTTATATATTTTGTAG
                                        −17 −19
AG = canonical 3′ splice site [symbol]
= cryptic 3′ splice site
A = SVM-BP predicted canonical branch point [symbol]
= SVM-BP predicted cryptic branch point
ABCB7-Forward primer sequence (SEQ ID NO: 7)
AATGAACAAAGCAGATAATGATGCAGG
ABCB7-Reverse primer sequence (SEQ ID NO: 8)
TCCCTGACTGGCGAGCACCATTA
PPOX-Forward primer sequence (SEQ ID NO: 9)
GGCCCTAATGGTGCTATCTTTG
PPOX-Reverse primer sequence (SEQ ID NO: 10)
CTTCTGAATCCAAGCCAAGCTC
```

1. A method of treating transfusion dependence in a patient with myelodysplastic syndrome (MDS), comprising administering a therapeutically effective amount of Compound 1 to the transfusion-dependent MDS patient who has an elevated ratio of aberrant junction to canonical junction TMEM14C transcripts (TMEM14C AJ/CJ ratio).

2. A method of treating transfusion dependence in a patient with MDS, comprising:
(a) determining that the transfusion-dependent MDS patient has an elevated TMEM14C AJ/CJ ratio; and
(b) administering a therapeutically effective amount of Compound 1 to the patient.

3. A method of identifying a transfusion-dependent MDS patient suitable for treatment with Compound 1, comprising:
(a) determining that the patient has an elevated TMEM14C AJ/CJ ratio; and
(b) identifying the patient as suitable for treatment with Compound 1.

4. A method of monitoring treatment efficacy in a transfusion-dependent MDS patient, comprising:
(a) determining that the patient has an elevated TMEM14C AJ/CJ ratio;
(b) administering a therapeutically effective amount of Compound 1 to the patient; and
(c) determining the TMEM14C AJ/CJ ratio in the patient after administration, wherein a reduction in the TMEM14C AJ/CJ ratio after administration indicates an effective treatment.

5. The method of claim 4, wherein the TMEM14C AJ/CJ ratio remains elevated after step (c), and the method further comprises administering an additional dose of Compound 1 to the patient.

6. The method of claim 4 or 5, wherein the method further comprises administering additional doses of Compound 1 to the patient until the TMEM14C AJ/CJ ratio is no longer elevated.

7. The method of any one of claims 1 to 6, wherein determining an elevated AJ/CJ ratio comprises obtaining a biological sample from the patient and determining a TMEM14C AJ/CJ ratio in the sample.

8. The method of claim 7, wherein the biological sample comprises a blood sample or a bone marrow sample.

9. The method of claim 8, wherein the blood sample comprises peripheral blood or plasma.

10. The method of claim 8, wherein the bone marrow sample comprises a bone marrow aspirate or a bone marrow biopsy.

11. The method of any one of claims 1 to 10, wherein the TMEM14C AJ/CJ ratio is determined by measuring RNA transcripts in the patient or in a biological sample from the patient.

12. The method of claim 11, wherein measuring RNA transcripts comprises nucleic acid barcoding and/or real-time polymerase chain reaction (RT-PCR).

13. The method of claim 11 or 12, wherein measuring RNA transcripts comprises nucleic acid barcoding.

14. The method of any one of claims 1 to 13, wherein an elevated TMEM14C AJ/CJ ratio is a ratio greater than about 0.1, about 0.2, about 0.5, about 1, about 2, about 4, about 10, about 15, about 20, or about 30, e.g., as measured by nucleic acid barcoding.

15. The method of any one of claims 1 to 14, wherein an elevated TMEM14C AJ/CJ ratio is a ratio greater than about 4, as measured by nucleic acid barcoding.

16. The method of any one of claims 1 to 15, wherein the method further comprises determining that the patient has an elevated ABCB7 AJ/CJ ratio.

17. The method of any one of claims 1 to 16, wherein the method further comprises determining that the patient has an elevated PPOX AJ/CJ ratio.

18. The method of any one of claims 1 to 17, wherein the method further comprises determining that the patient has an elevated ABCB7 AJ/CJ ratio and an elevated PPOX AJ/CJ ratio.

19. The method of any one of claims 1 to 18, wherein the MDS is MDS with multilineage dysplasia (MDS-MLD), MDS with single lineage dysplasia (MDS-SLD), MDS with ring sideroblasts (MDS-RS), MDS with excess blasts (MDS-EB), MDS associated with isolated del(5q), or MDS-unclassified (MDS-U).

20. The method of any one of claims 1 to 19, wherein the MDS is MDS of intermediate-1 risk or lower according to the International Prognostic Scoring System.

21. The method of any one of claims 1 to 20, wherein the MDS is MDS of intermediate-2 risk or higher according to the International Prognostic Scoring System.

22. The method of any one of claims 1 to 21, wherein the MDS is MDS-MLD.

23. The method of any one of claims 1 to 21, wherein the MDS is MDS-EB.

24. The method of claim 23, wherein the MDS-EB is MDS-EB1 or MDS-EB2.

25. The method of claim 23 or 24, wherein the MDS is MDS-EB2.

26. The method of any one of claims 1 to 25, wherein the patient or a biological sample from the patient comprises a mutation in one or more genes associated with RNA splicing.

27. The method of any one of claims 1 to 26, wherein the patient or a biological sample from the patient comprises a mutation in one or more genes selected from SF3B1, SRSF2, U2AF1, and ZRSR2.

28. The method of any one of claims 1 to 27, wherein the patient or a biological sample from the patient comprises a mutation in SF3B1.

29. The method of claim 28, wherein the mutation in SF3B1 comprises or consists of a mutation at one or more of positions E622, H662, K666, K700, R625, or V701 in SF3B1.

30. The method of claim 28 or 29, wherein the mutation in SF3B1 comprises or consists of a mutation at one or more of positions H662, K700, or R625 in SF3B1.

31. The method of any one of claims 28 to 30, wherein the mutation in SF3B1 comprises or consists of a mutation at position K700 in SF3B1.

32. The method of any one of claims 28 to 31, wherein the mutation in SF3B1 comprises K700E and/or R625C.

33. The method of any one of claims 1 to 32, wherein the patient or a biological sample from the patient comprises a low level of TMEM14C expression.

34. The method of any of claims 1, 2, or 4 to 33, wherein Compound 1 is administered to the patient orally.

35. The method of any of claims 1, 2, or 4 to 34, wherein Compound 1 is administered to the patient once daily.

36. The method of claim 35, wherein Compound 1 is administered to the patient once daily on a 5 days on/9 days off dosing schedule.

37. The method of claim 35, wherein Compound 1 is administered to the patient once daily on a 21 days on/7 days off dosing schedule.

38. The method of claim 35, wherein Compound 1 is administered to the patient once daily on a continuous dosing schedule.

39. The method of any one of claims 35 to 38, wherein Compound 1 is administered to the patient once daily for one or more 28-day cycles.

40. The method of any one of claims 35 to 39, wherein the therapeutically effective amount of Compound 1 is about 2 mg to about 20 mg given in a single dose on the day of administration.

41. The method of any one of claims 35 to 40, wherein the therapeutically effective amount of Compound 1 is about 2 mg, about 3.5 mg, about 5 mg, about 7 mg, about 10 mg, about 12 mg, about 14, or about 20 mg given in a single dose on the day of administration.

42. The method of any of claims 1, 2, or 4 to 34, wherein Compound 1 is administered to the patient twice daily.

43. The method of claim 42, wherein Compound 1 is administered to the patient twice daily on a 5 days on/9 days off dosing schedule.

44. The method of claim 42, wherein Compound 1 is administered to the patient twice daily on a 21 days on/7 days off dosing schedule.

45. The method of claim 42, wherein Compound 1 is administered to the patient twice daily on a continuous dosing schedule.

46. The method of any one of claims 42 to 45, wherein Compound 1 is administered to the patient twice daily for one or more 28-day cycles.

47. The method of any one of claims 42 to 46, wherein the therapeutically effective amount of Compound 1 is a total of about 2 mg to about 20 mg given in two divided doses on the day of administration.

48. The method of any one of claims 42 to 47, wherein the therapeutically effective amount of Compound 1 is about 10 mg, about 15 mg, or about 20 mg given in two divided doses on the day of administration.

49. The method of claim 47 or 48, wherein the first dose is about 10 mg and the second dose is about 5 mg.

50. The method of claim 47 or 48, wherein the first dose is about 5 mg and the second dose is about 10 mg.

51. The method of claim 47 or 48, wherein the first dose and the second dose are each about 5 mg.

52. The method of claim 47 or 48, wherein the first dose and the second dose are each about 7.5 mg.

53. The method of claim 47 or 48, wherein the first dose and the second dose are each about 10 mg.

54. The method of any one of claims 1 to 53, wherein treatment with Compound 1 reduces or eliminates the patient's transfusion dependence.

55. The method of any one of claims 1 to 54, wherein treatment with Compound 1 reduces the number or frequency of transfusions given to the patient by at least about 10%, about 20%, about 30%, about 40%, about 50%, or about 60% as compared to the number or frequency prior to treatment.

56. The method of any one of claims 1 to 55, wherein treatment with Compound 1 reduces the number or frequency of transfusions given to the patient by at least about 30% as compared to the number or frequency prior to treatment.

57. The method of any one of claims 1 to 56, wherein treatment with Compound 1 reduces the number or frequency of transfusions given to the patient by at least about 60% as compared to the number or frequency prior to treatment.

58. The method of any one of claims 1 to 57, wherein the patient does not receive any transfusions for a period of at least 56 consecutive days, wherein the period begins any time after the start of treatment.

59. The method of any one of claims 1 to 58, wherein the transfusions comprise red blood cell (RBC) and/or platelet transfusions.

60. The method of any one of claims 1 to 59, wherein the transfusions comprise RBC transfusions.

61. The method of any one of claims 1 to 60, wherein treatment with Compound 1 increases the amount of bone marrow sideroblasts in the patient as compared to the amount prior to treatment.

62. The method of any one of claims 1 to 61, wherein treatment with Compound 1 increases the amount of bone marrow sideroblasts in the patient by at least about 10%, about 20%, about 30%, or about 40% as compared to the amount prior to treatment.

63. A method of treating transfusion dependence in a patient with MDS, comprising administering a therapeutically effective amount of Compound 1 to the transfusion-dependent MDS patient who has an elevated ABCB7 AJ/CJ ratio.

64. A method of treating transfusion dependence in a patient with MDS, comprising:
(a) determining that the transfusion-dependent MDS patient has an elevated ABCB7 AJ/CJ ratio; and
(b) administering a therapeutically effective amount of Compound 1 to the patient.

65. A method of identifying a transfusion-dependent MDS patient suitable for treatment with Compound 1, comprising:
(a) determining that the patient has an elevated ABCB7 AJ/CJ ratio; and
(b) identifying the patient as suitable for treatment with Compound 1.

66. The method of any one of claims 63 to 65, wherein determining an elevated AJ/CJ ratio comprises obtaining a biological sample from the patient and determining an ABCB7 AJ/CJ ratio in the sample.

67. The method of any one of claims 63 to 66, wherein the ABCB7 AJ/CJ ratio is determined by measuring RNA transcripts in the patient or in a biological sample from the patient.

68. The method of claim 67, wherein measuring RNA transcripts comprises nucleic acid barcoding and/or real-time polymerase chain reaction (RT-PCR).

69. The method of claim 67 or 68, wherein measuring RNA transcripts comprises nucleic acid barcoding.

70. A method of treating transfusion dependence in a patient with MDS, comprising administering a therapeutically effective amount of Compound 1 to the transfusion-dependent MDS patient who has an elevated PPOX AJ/CJ ratio.

71. A method of treating transfusion dependence in a patient with MDS, comprising:
(a) determining that the transfusion-dependent MDS patient has an elevated PPOX AJ/CJ ratio; and
(b) administering a therapeutically effective amount of Compound 1 to the patient.

72. A method of identifying a transfusion-dependent MDS patient suitable for treatment with Compound 1, comprising:
(a) determining that the patient has an elevated PPOX AJ/CJ ratio; and
(b) identifying the patient as suitable for treatment with Compound 1.

73. The method of any one of claims 70 to 72, wherein determining an elevated AJ/CJ ratio comprises obtaining a biological sample from the patient and determining a PPOX AJ/CJ ratio in the sample.

74. The method of any one of claims 70 to 73, wherein the PPOX AJ/CJ ratio is determined by measuring RNA transcripts in the patient or in a biological sample from the patient.

75. The method of claim 74, wherein measuring RNA transcripts comprises nucleic acid barcoding and/or real-time polymerase chain reaction (RT-PCR).

76. The method of claim 74 or 75, wherein measuring RNA transcripts comprises nucleic acid barcoding.


SEQUENCE LISTING (10 sequences)

SEQ ID NO: 1 - DNA, 40 bp, Unknown
  source: /note=“Description of Unknown TMEM14C sequence”
ccggggccttcgtgagaccggtgcaggcctggggtagtct

SEQ ID NO: 2 - DNA, 40 bp, Unknown
  source: /note=“Description of Unknown TMEM14C sequence”
ccggggccttcgtgagaccgcttgttttctgcaggtgcag

SEQ ID NO: 3 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 4 - DNA, 6463 bp, Homo sapiens
agttccgtctgtgtgttcgagtggacaaaatggcgaagatcgccaagactcacgaagatattgaagcacagattcgagaaattcaaggcaagaaggcagctcttgatgaagctcaaggagtgggcctcgattctacaggttattatgaccaggaaatttatggtggaagtgacagcagatttgctggatacgtgacatcaattgctgcaactgaacttgaagatgatgacgatgactattcatcatctacgagtttgcttggtcagaagaagccaggatatcatgcccctgtggcattgcttaatgatataccacagtcaacagaacagtatgatccatttgctgagcacagacctccaaagattgcagaccgggaagatgaatacaaaaagcataggcggaccatgataatttccccagagcgtcttgatccttttgcagatggagggaaaacccctgatcctaaaatgaatgctaggacttacatggatgtaatgcgagaacaacacttgactaaagaagaacgagaaattaggcaacagctagcagaaaaagctaaagctggagaactaaaagtcgtcaatggagcagcagcgtcccagcctccatcaaaacgaaaacggcgttgggatcaaacagctgatcagactcctggtgccactcccaaaaaactatcaagttgggatcaggcagagacccctgggcatactccttccttaagatgggatgagacaccaggtcgtgcaaagggaagcgagactcctggagcaaccccaggctcaaaaatatgggatcctacacctagccacacaccagcgggagctgctactcctggacgaggtgatacaccaggccatgcgacaccaggccatggaggcgcaacttccagtgctcgtaaaaacagatgggatgaaacccccaaaacagagagagatactcctgggcatggaagtggatgggctgagactcctcgaacagatcgaggtggagattctattggtgaaacaccgactcctggagccagtaaaagaaaatcacggtgggatgaaacaccagctagtcagatgggtggaagcactccagttctgacccctggaaagacaccaattggcacaccagccatgaacatggctacccctactccaggtcacataatgagtatgactcctgaacagcttcaggcttggcggtgggaaagagaaattgatgagagaaatcgcccactttctgatgaggaattagatgctatgttcccagaaggatataaggtacttcctcctccagctggttatgttcctattcgaactccagctcgaaagctgacagctactccaacacctttgggtggtatgactggtttccacatgcaaactgaagatcgaactatgaaaagtgttaatgaccagccatctggaaatcttccatttttaaaacctgatgatattcaatactttgataaactattggttgatgttgatgaatcaacacttagtccagaagagcaaaaagagagaaaaataatgaagttgcttttaaaaattaagaatggaacaccaccaatgagaaaggctgcattgcgtcagattactgataaagctcgtgaatttggagctggtcctttgtttaatcagattcttcctctgctgatgtctcctacacttgaggatcaagagcgtcatttacttgtgaaagttattgataggatactgtacaaacttgatgacttagttcgtccatatgtgcataagatcctcgtggtcattgaaccgctattgattgatgaagattactatgctagagtggaaggccgagagatcatttctaatttggcaaaggctgctggtctggctactatgatctctaccatgagacctgatatagataacatggatgagtatgtccgtaacacaacagctagagcttttgctgttgtagcctctgccctgggcattccttctttattgcccttcttaaaagctgtgtgcaaaagcaagaagtcctggcaagcgagacacactggtattaagattgtacaacagatagctattcttatgggctgtgccatcttgccacatcttagaagtttagttgaaatcattgaacatggtcttgtggatgagcagcagaaagttcggaccatcagtgctttggccattgctgccttggctgaagcagcaactccttatggtatcgaatcttttgattctgtgttaaagcctttatggaagggtatccgccaacacagaggaaagggtttggctgctttcttgaaggctattgggtatcttattcctcttatggatgcagaatatgccaactactatactagagaagtgatgttaatccttattcgagaattccagtctcctgatgaggaaatgaaaaaaattgtgctgaaggtggtaaaacagtgttgtgggacagatggtgtagaagcaaactacattaaaacagagattcttcctcccttttttaaacacttctggcagcacaggatggctttggatagaagaaattaccgacagttagttgatactactgtggagttggcaaacaaagtaggtgcagcagaaattatatccaggattgtggatgatctgaaagatgaagccgaacagtacagaaaaatggtgatggagacaattgagaaaattatgggtaatttgggagcagcagatattgatcataaacttgaagaacaactgattgatggtattctttatgctttccaagaacagactacagaggactcagtaatgttgaacggctttggcacagtggttaatgctcttggcaaacgagtcaaaccatacttgcctcagatctgtggtacagttttgtggcgtttaaataacaaatctgctaaagttaggcaacaggcagctgacttgatttctcgaactgctgttgtcatgaagacttgtcaagaggaaaaattgatgggacacttgggtgttgtattgtatgagtatttgggtgaagagtaccctgaagtattgggcagcattcttggagcactgaaggccattgtaaatgtcataggtatgcataagatgactccaccaattaaagatctgctgcctagactcacccccatcttaaagaacagacatgaaaaagtacaagagaattgtattgatcttgttggtcgtattgctgacaggggagctgaatatgtatctgcaagagagtggatgaggatttgctttgagcttttagagctcttaaaagcccacaaaaaggctattcgtagagccacagtcaacacatttggttatattgcaaaggccattggccctcatgatgtattggctacacttctgaacaacctcaaagttcaagaaaggcagaacagagtttgtaccactgtagcaatagctattgttgcagaaacatgttcaccctttacagtactccctgccttaatgaatgaatacagagttcctgaactgaatgttcaaaatggagtgttaaaatcgctttccttcttgtttgaatatattggtgaaatgggaaaagactacatttatgccgtaacaccgttacttgaagatgctttaatggatagagaccttgtacacagacagacggctagtgcagtggtacagcacatgtcacttggggtttatggatttggttgtgaagattcgctgaatcacttgttgaactatgtatggcccaatgtatttgagacatctcctcatgtaattcaggcagttatgggagccctagagggcctgagagttgctattggaccatgtagaatgttgcaatattgtttacagggtctgtttcacccagcccggaaagtcagagatgtatattggaaaatttacaactccatctacattggttcccaggacgctctcatagcacattacccaagaatctacaacgatgataagaacacctatattcgttatgaacttgactatatcttataattttattgtttattttgtgtttaatgcacagctacttcacaccttaaacttgctttgatttggtgatgtaaacttttaaacattgcagatcagtgtagaactggtcatagaggaagagctagaaatccagtagcatgatttttaaataacctgtctttgtttttgatgttaaacagtaaatgccagtagtgaccaagaacacagtgattatatacactatactggagggatttcatttttaattcatctttatgaagatttagaactcattccttgtgtttaaagggaatgtttaattgagaaataaacatttgtgtacaaaatgctaatttgtgtgtgttttttgaacatgacttgtaaaatgcggaactttgataaagtattggtttatgtggaataagtggcttaatttcattttctgtcacatggtttatagaaagtagttagctgaataaaaactatataaagtgatggcatctttgtcaaaattccattgttgtgttaataatgacagcaagaagagtagcctcaggggatgttcccctcaaactagcacaaccattccatcatcgtagaaaagtagcacttttgctaaactgtcttgaatattttgtacttacatagcgcctttcatctcttgatttctcaaaatgctttatgaacacatttaaagaaagtggtttaagtcttgtccaacacttgacaggtctgctgtgtttagcaagtgaggaatttaactttacttcaaaactgctttctgcctattaggagtgaggatacctaagtaatgctgatagaacaggacaatgttgggcttttctccatgttataagccactactcagcaatgcatcagtaaatacctattcacccactgtatgccagccactgtgcttatatgcaggggatgcaaaggtgcatgagacctgccctctacctttaagaacagtataatggagaaggggagacaaaccttgtgacagattccttatgtactgtgataatacacagtggcagcagctaaaatgtctaggtttgcttagctttgtattcagtaataataagttgatctaagagttcagcataaactgaatgaaatgccatttaatggtagaggaaccaagcattaaggcagtactacacttaattttttaagcaaatatgtaaagtatattttcaaacttttctaatgttatggccccaaatttcttagttttggcctttttactacctatactatttttactgttttgttttgtctccatgtggtagtacttctgtgaactctaaagggaaaaaaaattctgcaagcagcattagtatataactactactgtaagtaaaactgcctattgacactttaggagttcctgcttcagaagcttagttaagaaacagcttgtggccgggtgtggtggctcatgcctgtaatcccagcactttgggaggctgaggcgggcggatcacgaggtcaggagatcgagaccatcctggctaacgcggtgaagtcccgtctactaaaaatacaaaaattagccaggcgtggtggcgggcgcctgtagtcccagctactagggaggctgaggcaggagaatggcttgaacccgggaggcggagcttgcagtgagccgagatagcgctgctacactccagcctgggcgacagagactccgtctcaaaaaaaaaaaaaaaattgactttcaacaaattgatagtgagcattaagggtttccaagttggatttgtaactcctcatcattccttgtatgacaactttctgaatatatgtcactatgtagtaaaattaaacactccaaactcatctttctgttgttagaagttttcagcggtacttccatgcaactttaaatctcactgctctctatggttgatgtcaaatgaccttcagtaatgactgagaattgaatacaaatagattacaaagccaaaatttgatgttaaatgactcaggaaattttagttgtattttcaattcaagtacttagtagcctacgtttgcttggcctctggttctttatggaaaataggctttgtagtggcattgtggagcaaaggagactgttacaccttaattaactttttttactgatgcaaataatttgaggatagagaggagggaagtagtgaaagctatgacctaaaacattgggaccaaatagaggctcacagatatttggattattttatgtgcttattattaaataaggaaagcattttgtgatatgtggaagacgctatgtgaagttttacctatcttctcaaagaccttttcttttgtattttcttttggtgtttcttaaagccaaacaaagaaatgttcttaaggagacagggtgggtttttctgtgggcctttgttggtttttctgtgggccatcgccctctaatggaattgatctctggctgtttgatttttttcatattgtatttttaaaatttgttgtacagtgccctgtgagcaccaagtaccactagatgaataaaacgtattatatctaaa

SEQ ID NO: 5 - DNA, 56 bp, Unknown
  source: /note=“Description of Unknown TMEM14C sequence”
ctgactgctggagagctgtgcttttaactacctctgatccagcttgttttctgcag

SEQ ID NO: 6 - DNA, 59 bp, Unknown
  source: /note=“Description of Unknown ABCB7 sequence”
cacttaagagaataaattctaattatacttgcctatagttatttttatatattttgtag

SEQ ID NO: 7 - DNA, 27 bp, Artificial Sequence
  source: /note=“Description of Artificial Sequence Synthetic primer”
aatgaacaaagcagataatgatgcagg

SEQ ID NO: 8 - DNA, 23 bp, Artificial Sequence
  source: /note=“Description of Artificial Sequence Synthetic primer”
tccctgactggcgagcaccatta

SEQ ID NO: 9 - DNA, 22 bp, Artificial Sequence
  source: /note=“Description of Artificial Sequence Synthetic primer”
ggccctaatggtgctatctttg

SEQ ID NO: 10 - DNA, 22 bp, Artificial Sequence
  source: /note=“Description of Artificial Sequence Synthetic primer”
cttctgaatccaagccaagctc